(19) United States
(12) Patent Application Publication
(10) Pub. No.: US 2020/0231964 A1
(43) Pub. Date: Jul. 23, 2020

(54) TAGGING NUCLEIC ACID MOLECULES FROM SINGLE CELLS FOR PHASED SEQUENCING

(71) Applicant: MetaBiotech Corporation, San Jose, CA (US)

(72) Inventors: Tuval Ben-Yehezkel, Sunnyvale, CA (US); Indira Wu, San Carlos, CA (US)

(21) Appl. No.: 16/783,301

(22) Filed: Feb. 6, 2020

Related U.S. Application Data
(63) Continuation of application No. PCT/US2018/046356, filed on Aug. 10, 2018.
(60) Provisional application No. 62/543,687, filed on Aug. 10, 2017.

(51) Int. Cl. C12N 15/10; C12Q 1/6874

(52) U.S. Cl. CPC C12N 15/1065; C12Q 1/6874; C12N 15/1096

(57) ABSTRACT

The present disclosure provides methods for long-read sequencing from single cells. The method can comprise constructing a nucleic acid library and reconstructing longer nucleic acid sequences by clustering and assembling a plurality of shorter nucleic acid sequences.

### CROSS REFERENCE

This application is a continuation of International Patent Application No. PCT/US2018/046356, filed Aug. 10, 2018, which claims the benefit of U.S. Provisional Application No. 62/543,687, filed Aug. 10, 2017, each of which is incorporated herein by reference in its entirety.

### SEQUENCE LISTING

The present disclosure contains a Sequence Listing which has been submitted electronically in ASCII format and is hereby incorporated by reference in its entirety. Said ASCII copy, created on Aug. 9, 2018, is named 50112-705_601_SL.txt and is 41,162 bytes in size.

### BACKGROUND

Over the last decade, advances in Next Generation Sequencing (NGS) technologies have allowed researchers to resequence genomes, epigenomes, and transcriptomes, and have revolutionized the molecular diagnosis of human genetic diseases. The throughput and accuracy of Next Generation Sequencing allows for identification of small and large-scale variations, ranging from a single nucleotide substitution for genome sequencing, to deoxyribonucleic acid (DNA) methylation pattern for epigenome sequencing, to gene expression profile using transcriptome sequencing (ribonucleic acid (RNA) sequencing). Until recently, most of these resequencing efforts focus on biological samples where the nucleic acid contents were extracted from tissues or cell ensembles. While high throughput sequencing allows for detailed analysis and correlation between phenotypes and genomic variations, the analysis represents the ensembled measurements of the analyzed sample and masks the many subtleties that can exist amongst even cells of the same cell type. The ensembled behavior of a cell population may not represent the behavior of individual cells. Different temporal positioning in the cell cycle, different spatial positioning within the tissue, somatic mutations and stochastic gene expression can all contribute to the difference in expression levels between cells within a population. In addition, the ensembled measurement of the cell population can mask the presence of a subpopulation of cells with disproportional influence over the larger population. Such is the case with tumor tissues and microbial populations, which are notoriously heterogeneous, both in terms of the composition of the cell population and the clonal evolution of the cells, and have dynamic responses to therapeutic treatments. Understanding the heterogeneity within cancer cell populations can provide invaluable insights into the complex intercellular interactions that govern tumor behavior and microbiomes, and are important to individualized care.

### SUMMARY

In some aspects, the present disclosure provides a method comprising: (a) providing a plurality of nucleic acid molecules from a single cell inside a partition; (b) appending an adapter to an end of said plurality of nucleic acid molecules inside said partition, wherein said adapter comprises a partition-specific barcode and a molecule-specific barcode, thereby generating a plurality of barcoded nucleic acid molecules, wherein said partition-specific barcode is common to each of said plurality of barcoded nucleic acid molecules inside said partition; (c) amplifying said plurality of barcoded nucleic acid molecules, thereby generating a plurality of amplified barcoded nucleic acid molecules; (d) fragmenting said plurality of amplified barcoded nucleic acid molecules to generate a plurality of nucleic acid fragments, wherein at least a portion of (e.g., each of) the nucleic acid fragments from at least a portion of (e.g., each of) said plurality of nucleic acid fragments comprises a first end without said adapter and a second end comprising said adapter; and (e) circularizing said plurality of nucleic acid fragments by ligating said first end to said second end of at least a portion of (e.g., each of) said nucleic acid fragments from said plurality of nucleic acid fragments, thereby generating a plurality of circularized nucleic acid molecules comprising said adapter.

In some embodiments, the method further comprises sequencing said plurality of circularized nucleic acid molecules to generate sequencing reads. In some embodiments, the method further comprises clustering said sequencing reads using said molecule-specific barcodes to generate long read sequencing information for said plurality of nucleic acid molecules from said single cell. In some embodiments, the method further comprises encapsulating said single cell inside said partition prior to (a). In some embodiments, the method further comprises extracting said plurality of nucleic acid molecules inside said partition. In some embodiments, said plurality of nucleic acid molecules from said single cell comprises deoxyribonucleic acid (DNA). In some embodiments, said plurality of nucleic acid molecules from said single cell comprises complementary deoxyribonucleic acid (DNA). In some embodiments, said plurality of nucleic acid molecules from said single cell comprises RNA. In some embodiments, said adapter is appended to a 5′ end and a 3′ end of said plurality of nucleic acid molecules. In some embodiments, said fragmenting comprises randomly fragmenting said amplified barcoded nucleic acid molecules. In some embodiments, the method further comprises phasing said sequencing reads to determine a molecular origin of two or more alleles in said plurality of nucleic acid molecules. In some embodiments, at least a portion of (e.g., each of) said plurality of barcoded nucleic acid molecules comprises a unique molecule-specific barcodes. In some embodiments, a separate long read sequence is generated for each of said unique molecule-specific barcodes. In some embodiments, a long read sequence is generated for said unique molecule-specific barcodes (each of said unique molecule-specific barcodes). In some embodiments, the method further comprises performing (a) to (e) in a plurality of partitions, wherein each partition comprises a plurality of nucleic acid molecules from a single cell. In some embodiments, the method further comprises differentiating between sequence reads from different partitions based on said partition-specific barcode. In some embodiments, the method comprises sequencing said plurality of barcoded nucleic acid molecules to generate sequence reads and differentiating between sequence reads from different partitions based on said partition-specific barcode.

In some aspects, the present disclosure provides a method comprising: (a) providing a plurality of nucleic acid molecules from a single cell inside a partition; (b) appending said plurality of nucleic acid molecules inside said partition with a partition-specific barcode on a first end and a molecule-specific barcode on a second end, thereby generating a plurality of barcoded nucleic acid molecules comprising said partition-specific barcode and said molecule-specific barcode on opposing ends, wherein said partition-specific barcode is common to each of said plurality of barcoded nucleic acid molecules inside said partition; (c) amplifying said plurality of barcoded nucleic acid molecules, thereby generating a plurality of amplified barcoded nucleic acid molecules; (d) fragmenting said plurality of amplified barcoded nucleic acid molecules to generate a first plurality of nucleic acid fragments comprising a first end comprising said molecule-specific barcode and a second end without said molecule-specific barcode, and a second plurality of nucleic acid fragments comprising a first end comprising said partition-specific barcode and a second end without said partition-specific barcode; and (e) circularizing said plurality of nucleic acid fragments by ligating said first end to said second end in at least a portion of (e.g., each of) said first plurality of nucleic acid fragments, thereby generating a plurality of circularized nucleic acid molecules comprising said molecule-specific barcode.

In some embodiments, the method further comprises sequencing said plurality of circularized nucleic acid molecules to generate sequencing reads. In some embodiments, the method further comprises clustering said sequencing reads using said molecule-specific barcodes to generate long read sequencing information for said plurality of nucleic acid molecules from said single cell. In some embodiments, the method further comprises encapsulating said single cell inside said partition prior (a). In some embodiments, the method further comprises extracting said plurality of nucleic acid molecules inside said partition. In some embodiments, said plurality of nucleic acid molecules from said single cell comprises DNA. In some embodiments, said plurality of nucleic acid molecules from said single cell comprises cDNA. In some embodiments, said plurality of nucleic acid molecules from said single cell comprises RNA. In some embodiments, said fragmenting comprises randomly fragmenting said amplified barcoded nucleic acid molecules. In some embodiments, the method further comprises phasing said sequencing reads to determine a molecular origin of two or more alleles in said plurality of nucleic acid molecules. In some embodiments, at least a portion of (e.g., each of) said plurality of barcoded nucleic acid molecules comprises a unique molecule-specific barcode. In some embodiments, a separate long read sequence is generated in for each of said unique molecule-specific barcodes. In some embodiments, a long read sequence is generated for said unique molecule-specific barcodes (generated for each unique molecule-specific barcodes). In some embodiments, the method further comprises performing (a) to (e) in a plurality of partitions, wherein each partition comprises a plurality of nucleic acid molecules from a single cell. In some embodiments, the method further comprises differentiating between sequence reads from different partitions based on said partition-specific barcode. In some embodiments, the method further comprises sequencing said plurality of barcoded nucleic acid molecules to generate sequence reads and differentiating between sequence reads from different partitions based on said partition-specific barcode.

In some aspects, the present disclosure provides a method comprising: (a) providing a plurality of nucleic acid molecules from a single cell inside a partition; (b) appending said plurality of nucleic acid molecules inside said partition with a partition-specific barcode on a first end and a molecule-specific barcode on a second end, thereby generating a plurality of barcoded nucleic acid molecules comprising said partition-specific barcode and said molecule-specific barcode on opposing ends, wherein said partition-specific barcode is common to each of said plurality of barcoded nucleic acid molecules inside said partition; (c) amplifying said plurality of barcoded nucleic acid molecules, thereby generating a plurality of amplified barcoded nucleic acid molecules; (d) fragmenting said plurality of amplified barcoded nucleic acid molecules, thereby generating a first population of nucleic acid fragments comprising said partition-specific barcode and a second population of nucleic acid fragments comprising said molecule-specific barcode; (e) ligating said first population of nucleic acid fragments and said second population of nucleic acid fragments, thereby generating a plurality of ligated nucleic acid fragments, wherein at least a portion of (e.g., each of) said plurality of ligated nucleic acid fragments comprises said partition-specific barcode and said molecule-specific barcode adjacent to each other within said ligated nucleic acid fragment; and (f) circularizing said plurality of nucleic acid fragments by ligating opposing ends of at least a portion of (e.g., each of) said plurality of ligated nucleic acid fragments, thereby generating a plurality of circularized nucleic acid molecules.

In some embodiments, the method further comprises sequencing said plurality of circularized nucleic acid molecules to generate sequencing reads. In some embodiments, the method further comprises pairing said molecule-specific barcode and said partition-specific barcode from said sequencing reads to generate long read sequencing information for said plurality of nucleic acid molecules from said single cell. In some embodiments, the method further comprises performing (a) to (f) in a plurality of partitions, wherein each partition comprises a plurality of nucleic acid molecules from a single cell. In some embodiments, the method further comprises differentiating between sequence reads from different partitions based on said partition-specific barcode. In some embodiments, the method further comprises sequencing said plurality of barcoded nucleic acid molecules to generate sequence reads and differentiating between sequence reads from different partitions based on said partition-specific barcode. In some embodiments, the method further comprises encapsulating said single cell inside said partition prior to (a). In some embodiments, the method further comprises extracting said plurality of nucleic acid molecules inside said partition. In some embodiments, said plurality of nucleic acid molecules from said single cell comprises DNA. In some embodiments, said plurality of nucleic acid molecules from said single cell comprises cDNA. In some embodiments, said plurality of nucleic acid molecules from said single cell comprises RNA. In some embodiments, said fragmenting comprises randomly fragmenting said amplified barcoded nucleic acid molecules. In some embodiments, the method further comprises phasing said sequencing reads to determine a molecular origin of two or more alleles in said plurality of nucleic acid molecules. In some embodiments, at least a portion of (e.g., each of) said plurality of barcoded nucleic acid molecules comprises a unique molecule-specific barcode. In some embodiments, a separate pairing is generated for said unique molecule-specific barcode (generated for each of said unique molecule-specific barcode). In some embodiments, the method comprises pairing each of said unique molecule-specific barcode.

In some aspects, the present disclosure provides a method comprising: (a) providing a plurality of nucleic acid molecules from a single cell inside a partition; (b) appending an adapter to an end of said plurality of nucleic acid molecules inside said partition, wherein said adapter comprises a partition-specific barcode and a molecule-specific barcode, thereby generating a plurality of barcoded nucleic acid molecules, wherein said partition-specific barcode is common to each of said plurality of barcoded nucleic acid molecules inside said partition; (c) amplifying said plurality of barcoded nucleic acid molecules, thereby generating a plurality of amplified barcoded nucleic acid molecules; (d) appending an elongation sequence to at least a portion of (e.g., each of) said plurality of amplified barcoded nucleic acid molecules at said end comprising said adapter to generate a plurality of amplified barcoded nucleic acid molecules comprising said elongation sequence, wherein said elongation sequence comprises a sequence capable of annealing to a portion of (e.g., each of) a nucleic acid in at least a portion of (e.g., each of) said plurality of amplified barcoded nucleic acid molecules; (e) annealing said elongation sequence to said portion of said nucleic acid in said at least a portion of (e.g., each of) said plurality of amplified barcoded nucleic acid molecules; and (f) extending said elongation sequence annealed to said portion of said nucleic acid in said at least a portion of (e.g., each of) said plurality of amplified barcoded nucleic acid molecules with a polymerase thereby generating a plurality of extension products.

In some embodiments, the method further comprises sequencing said plurality of extension products to generate sequencing reads. In some embodiments, the method further comprises clustering said sequencing reads using said molecule-specific barcodes to generate long read sequencing information for said plurality of nucleic acid molecules from said single cell. In some embodiments, the method further comprises encapsulating said single cell inside said partition prior to (a). In some embodiments, the method further comprises extracting said plurality of nucleic acid molecules inside said partition. In some embodiments, said plurality of nucleic acid molecules from said single cell comprises DNA. In some embodiments, said plurality of nucleic acid molecules from said single cell comprises cDNA. In some embodiments, said plurality of nucleic acid molecules from said single cell comprises RNA. In some embodiments, the method further comprises fragmenting said amplified barcoded nucleic acid molecules. In some embodiments, said fragmenting comprises randomly fragmenting said amplified barcoded nucleic acid molecules. In some embodiments, the method further comprises phasing said sequencing reads to determine a molecular origin of two or more alleles in said plurality of nucleic acid molecules. In some embodiments, at least a portion of (e.g., each of) said plurality of barcoded nucleic acid molecules comprises a unique molecule-specific barcode. In some embodiments, a long read sequence is generated for said unique molecule-specific barcode (generated for each said unique molecule-specific barcode). In some embodiments, the method further comprises denaturing said plurality of amplified barcoded nucleic acid molecules comprising said elongation sequence prior to (e) to generate a plurality of single-stranded amplified barcoded nucleic acid molecules comprising said elongation sequence.

In some aspects, the present disclosure provides a method comprising: (a) providing a plurality of nucleic acid molecules from a single cell inside a partition; (b) appending said plurality of nucleic acid molecules inside said partition with a partition-specific barcode on a first end and a molecule-specific barcode on a second end, thereby generating a plurality of barcoded nucleic acid molecules comprising said partition-specific barcode and said molecule-specific barcode on opposing ends, wherein said partition-specific barcode is common to each of said plurality of barcoded nucleic acid molecules inside said partition; (c) amplifying said plurality of barcoded nucleic acid molecules, thereby generating a plurality of amplified barcoded nucleic acid molecules; (d) appending an elongation sequence to one or more ends of at least a portion of (e.g., each of) said plurality of amplified barcoded nucleic acid molecules to generate a plurality of amplified barcoded nucleic acid molecules comprising said elongation sequence, wherein said elongation sequence comprises a sequence capable of annealing to a portion of (e.g., each of) a nucleic acid in said at least a portion of (e.g., each of) said plurality of amplified barcoded nucleic acid molecules; (e) annealing said elongation sequence to said portion of said nucleic acid in said at least a portion of (e.g., each of) said plurality of amplified barcoded nucleic acid molecules; and (f) extending said elongation sequence annealed to said portion of said nucleic acid in at least a portion of (e.g., each of) said plurality of amplified barcoded nucleic acid molecules with a polymerase, thereby generating a plurality of extension products.

In some embodiments, the method further comprises sequencing said plurality of extension products to generate sequencing reads. In some embodiments, the method further comprises clustering said sequencing reads using said molecule-specific barcodes to generate long read sequencing information for said plurality of nucleic acid molecules from said single cell. In some embodiments, the method further comprises denaturing said plurality of amplified barcoded nucleic acid molecules comprising said elongation sequence prior to (e) to generate a plurality of single-stranded amplified barcoded nucleic acid molecules comprising said elongation sequence.

In some embodiments, said appending in (b) is performed by primer extension. In some embodiments, said plurality of nucleic acid molecules in (a) comprises RNA and said appending in (b) is performed by reverse transcription. In some embodiments, said appending in (b) is performed by ligation. In some embodiments, the method further comprises fragmenting said plurality of nucleic acid molecules prior to (b). In some embodiments, the method further comprises amplifying said plurality of nucleic acid molecules prior to (b). In some embodiments, said appending in (b) is performed inside said partition. In some embodiments, said amplifying is performed by PCR. In some embodiments, said partition-specific barcode and said molecule-specific barcode are immobilized on microparticles, wherein each microparticle comprises a plurality of identical partition-specific barcodes and a plurality of unique molecule-specific barcodes. In some embodiments, said partition comprises said microparticles. In some embodiments, said partition further comprises cell lysis buffer. In some embodiments, said partition is an aqueous droplet. In some embodiments, said partition comprises a single microparticle and a single cell. In some embodiments, said partition is formed by fusing a droplet comprising said nucleic acid from said single cell with a droplet comprising said partition-specific barcode and said molecule-specific barcode.

In some aspects, the present disclosure provides a method comprising: (a) appending a first terminal tag to a first end and a second terminal tag to a second end of at least a portion of (e.g., each of) a plurality of nucleic acid molecules to generate a plurality of barcoded nucleic acid molecules, wherein said first terminal tag comprises a first sequencing adapter sequence, a universal polymerase chain reaction (PCR) sequence, a partition-specific barcode, and a molecule-specific barcode, with or without a target molecule sequence, wherein said second terminal tag comprises a universal PCR sequence, with or without a target molecule sequence; (b) amplifying said plurality of barcoded nucleic acid molecules to generate amplified nucleic acid molecules; (c) fragmenting said amplified nucleic acid molecules, thereby generating a first plurality of barcoded fragments comprising a first end comprising said first terminal tag and a second end without said first terminal tag, and a second plurality of barcoded fragments comprising a first end comprising said second terminal tag and a second end without said second terminal tag; (d) circularizing said first plurality of barcoded fragments to generate circularized nucleic acid molecules; (e) fragmenting said circularized nucleic acid molecules, thereby generating a plurality of linear barcoded nucleic acid molecules, wherein said first terminal tag is in an internal region of at least a portion of (e.g., each of) said plurality of linear barcoded nucleic acid molecules; (f) appending a second sequencing adapter to each end of at least a portion of (e.g., each of) said plurality of linear barcoded nucleic acid molecules to generate a plurality of double adapter-ligated barcoded nucleic acid fragments; and (g) amplifying said plurality of double adapter-ligated barcoded nucleic acid fragments to generate a plurality of amplified double adapter-ligated barcoded nucleic acid fragments.

In some embodiments, the method further comprises sequencing said plurality of amplified double adapter-ligated barcode-tagged nucleic acid fragments to generate sequencing reads. In some embodiments, the method further comprises clustering said sequencing reads using said molecule-specific barcodes to generate long read sequencing information for said plurality of nucleic acid molecules. In some embodiments, said target molecule sequence on said first terminal tag comprises poly-thymine repeats and said target molecule sequence on said second terminal tag comprises poly-guanine repeats. In some embodiments, said target molecule sequence on said first terminal tag comprises a gene-specific sequence bracketing one end of a region of interest and said target molecule sequence on said second terminal tag comprises poly-guanine repeats. In some embodiments, said target molecule sequence on said first terminal tag comprises a gene-specific sequence bracketing one end of a region of interest and said target molecule sequence on said second terminal tag comprises a second gene-specific sequence bracketing the other end of said region of interest. In some embodiments, said target molecule sequence on said first terminal tag comprises poly-guanine repeats and said target molecule sequence on said second terminal tag comprises poly-thymine repeats. In some embodiments, said target molecule sequence on said first terminal tag comprises poly-thymine repeats. In some embodiments, said target molecule sequence on said first terminal tag comprises target-specific sequence. In some embodiments, said target molecule sequence on said first terminal tag comprises a random sequence of a length of at least 6 bases. In some embodiments, said target molecule sequence on said first terminal tag comprises a random sequence of a length of at least 8 bases. In some embodiments, said target molecule sequence on said first terminal tag comprises a random sequence of a length of at least 10 bases. In some embodiments, said target molecule sequence on said first terminal tag comprises a random sequence of a length of at least 12 bases. In some embodiments, said target molecule sequence on said first terminal tag comprises a random sequence of a length of at least 16 bases. In some embodiments, said target molecule sequence on said first terminal tag comprises a random sequence of a length of at least 20 bases.

In some aspects, the present disclosure provides a method comprising: (a) appending a first terminal tag comprising a universal polymerase chain reaction (PCR) sequence and a partition-specific barcode, with or without a target molecule sequence to a first end of a plurality of nucleic acid molecules; (b) appending a second terminal tag to a second end of said plurality of nucleic acid molecules, wherein said second terminal tag comprises a sequencing adapter sequence, a universal PCR sequence, and a molecule-specific barcode, with or without a target molecule sequence, thereby generating a plurality of barcoded nucleic acid molecules comprising a first terminal tag on a first end and a second terminal tag on a second end; (c) amplifying said plurality of barcoded nucleic acid molecules to generate amplified barcoded nucleic acid molecules; (d) fragmenting said amplified barcoded nucleic acid molecules, thereby generating a first plurality of barcoded fragments comprising a first end comprising said first terminal tag and a second end without said first terminal tag, and a second plurality of barcoded fragments comprising a first end comprising said second terminal tag and a second end without said second terminal tag; (e) circularizing said first and second plurality of barcoded fragments to generate circularized nucleic acid molecules; (f) fragmenting said circularized nucleic acid molecules, thereby generating a plurality of linear barcoded nucleic acid molecules, wherein said first terminal tag is in an internal region of at least a portion of (e.g., each of) said plurality of linear barcoded nucleic acid molecules; (g) appending a second sequencing adapter to each end of at least a portion of (e.g., each of) said plurality of linear barcoded nucleic acid molecules to generate a plurality of double adapter-ligated barcoded nucleic acid fragments; and (h) amplifying said plurality of double adapter-ligated barcoded nucleic acid fragments to generate a plurality of amplified double adapter-ligated barcoded nucleic acid fragments.

In some embodiments, the method further comprises sequencing said plurality of amplified double adapter-ligated barcode-tagged nucleic acid fragments to generate sequencing reads. In some embodiments, the method further comprises clustering said sequencing reads using said molecule-specific barcodes to generate long read sequencing information for said plurality of nucleic acid molecules. In some embodiments, said target molecule sequence on said partition-specific barcode tag comprises poly-thymine repeats and said target molecule sequence on said molecule-specific tag comprises poly-guanine repeats. In some embodiments, said target molecule sequence on said partition-specific barcode tag comprises a target-specific sequence bracketing one end of a region of interest and said target molecule sequence on said molecule-specific tag comprises poly-guanine repeats. In some embodiments, said target molecule sequence on said partition-specific barcode tag comprises a target-specific sequence bracketing one end of a region of interest and said target molecule sequence on said molecule-specific tag comprises a second gene-specific sequence bracketing the other end of said region of interest. In some embodiments, said target molecule sequence on said partition-specific barcode tag comprises poly-guanine repeats and said target molecule sequence on said molecule-specific barcode tag comprises poly-thymine repeats. In some embodiments, said target molecule sequence on said partition-specific barcode tag comprises a poly-thymine repeats. In some embodiments, said target molecule sequence on said partition-specific barcode tag comprises a gene-specific sequence. In some embodiments, said target molecule sequence on said partition-specific barcode tag comprises a random sequence of a length of at least 6 bases. In some embodiments, said target molecule sequence on said partition-specific barcode tag comprises a random sequence of a length of at least 8 bases. In some embodiments, said target molecule sequence on said partition-specific barcode tag comprises a random sequence of a length of at least 10 bases. In some embodiments, said target molecule sequence on said partition-specific barcode tag comprises a random sequence of a length of at least 12 bases. In some embodiments, said target molecule sequence on said partition-specific barcode tag comprises a random sequence of a length of at least 16 bases. In some embodiments, said target molecule sequence on said partition-specific barcode tag comprises a random sequence of a length of at least 20 bases. In some embodiments, said appending in (b) takes place inside single-cell partitions. In some embodiments, said appending in (b) takes place after partitions are broken and all said barcode-tagged nucleic acid molecules are pooled. In some embodiments, said appending in (b) is performed by primer extension. In some embodiments, said appending in (b) is performed by ligation. In some embodiments, said nucleic acid molecules are fragmented prior to appending with molecule-specific barcode in (b). In some embodiments, said amplifying in (c) is performed by PCR

In some embodiments, said appending in (a) takes place inside a partition. In some embodiments, said appending in (a) is performed by primer extension. In some embodiments, said appending in (a) is performed by reverse transcription. In some embodiments, said appending in (a) is performed by ligation.

In some aspects, the present disclosure provides a method comprising: (a) appending a first terminal tag to a first end and a second terminal tag to a second end of at least a portion of (e.g., each of) a plurality of nucleic acid molecules to generate a plurality of barcoded nucleic acid molecules, wherein said first terminal tag comprises a first sequencing adapter sequence, a universal polymerase chain reaction (PCR) sequence, a partition-specific barcode, and a molecule-specific barcode, with or without a target molecule sequence, wherein said second terminal tag comprises a universal polymerase chain reaction (PCR) sequence, with or without a target molecule sequence; (b) amplifying said plurality of barcoded nucleic acid molecules, thereby generating a plurality of amplified barcoded nucleic acid molecules; (c) appending an elongation sequence to at least a portion of (e.g., each of) said plurality of amplified barcoded nucleic acid molecules at an end comprising said first terminal tag to generate a plurality of amplified barcoded nucleic acid molecules comprising said elongation sequence, wherein said elongation sequence comprises a sequence capable of annealing to a portion of (e.g., each of) a nucleic acid molecule in said at least a portion of (e.g., each of) said plurality of amplified barcoded nucleic acid molecules; (d) denaturing said plurality of amplified barcoded nucleic acid molecules comprising said elongation sequence to generate a plurality of single-stranded amplified barcoded nucleic acid molecules comprising said elongation sequence; (e) annealing said elongation sequence to said portion of said nucleic acid in at least a portion of (e.g., each of) said plurality of single-stranded amplified barcoded nucleic acid molecules; (f) extending said elongation sequence annealed to said portion of said nucleic acid in said at least a portion of (e.g., each of) said plurality of single-stranded amplified barcoded nucleic acid molecules with a polymerase thereby generating a plurality of extension products; (g) appending a second sequencing adapter to each end of at least a portion of (e.g., each of) said plurality of extension products to generate a plurality of double adapter barcoded nucleic acid fragments; and (h) amplifying said plurality of double adapter barcoded nucleic acid fragments to generate a plurality of amplified double adapter barcoded nucleic acid fragments.

In some embodiments, the method further comprises sequencing said plurality of amplified double adapter barcode-tagged nucleic acid fragments to generate sequencing reads. In some embodiments, the method further comprises clustering said sequencing reads using said molecule-specific barcodes to generate long read sequencing information for said plurality of nucleic acid molecules. In some embodiments, said amplifying in (b) is performed by PCR. In some embodiments, said appending in (c) is performed by PCR. In some embodiments, said appending in (c) is performed by ligation. In some embodiments, said appending in (g) is performed by PCR by using primers that contain said second sequencing adapter and a target-specific sequence downstream of said elongation sequence. In some embodiments, the method further comprises fragmenting said barcode-tagged and elongated nucleic acid molecules prior to said appending in (g).

In some aspects, the present disclosure provides a method comprising: (a) appending a first terminal tag comprising a universal polymerase chain reaction (PCR) sequence and a partition-specific barcode, with or without a target molecule sequence to a first end of a plurality of nucleic acid molecules; (b) appending a second terminal tag to a second end of said plurality of nucleic acid molecules, wherein said second terminal tag comprises a sequencing adapter sequence, a universal PCR sequence, and a molecule-specific barcode, with or without a target molecule sequence, thereby generating a plurality of barcoded nucleic acid molecules comprising a first terminal tag on a first end and a second terminal tag on a second end; (c) amplifying said plurality of barcoded nucleic acid molecules to generate amplified barcoded nucleic acid molecules; (d) appending an elongation sequence to an end of at least a portion of (e.g., each of) said plurality of amplified barcoded nucleic acid molecules to generate a plurality of amplified barcoded nucleic acid molecules comprising said elongation sequence, wherein said elongation sequence comprises a sequence capable of annealing to a portion of (e.g., each of) a nucleic acid molecule in said at least a portion of (e.g., each of) said plurality of amplified barcoded nucleic acid molecules; (e) denaturing said plurality of amplified barcoded nucleic acid molecules comprising said elongation sequence to generate a plurality of single-stranded amplified barcoded nucleic acid molecules comprising said elongation sequence; (f) annealing said elongation sequence to said portion of said nucleic acid in at least a portion of (e.g., each of) said plurality of single-stranded amplified barcoded nucleic acid molecules; (g) extending said elongation sequence annealed to said portion of said nucleic acid in said at least a portion of (e.g., each of) said plurality of single-stranded amplified barcoded nucleic acid molecules with a polymerase thereby generating a plurality of extension products; (h) appending a second sequencing adapter to each end of at least a portion of (e.g., each of) said plurality of extension products to generate a plurality of double adapter barcoded nucleic acid fragments; and (i) amplifying said plurality of double adapter barcoded nucleic acid fragments to generate a plurality of amplified double adapter-ligated barcoded nucleic acid fragments.

In some embodiments, the method further comprises sequencing said plurality of amplified double adapter-ligated barcode-tagged nucleic acid fragments to generate sequencing reads. In some embodiments, the method further comprises clustering said sequencing reads using said molecule-specific barcodes to generate long read sequencing information for said plurality of nucleic acid molecules. In some embodiments, said appending in (b) takes place inside a single-cell partition. In some embodiments, said appending in (b) takes place after partitions are broken and all said barcode-tagged nucleic acid molecules are pooled. In some embodiments, said appending in (b) is performed by primer extension. In some embodiments, said appending in (b) is performed by ligation. In some embodiments, said nucleic acid molecules are fragmented prior to said appending in (b). In some embodiments, said amplifying in (c) is performed by PCR. In some embodiments, said appending in (d) is performed by PCR. In some embodiments, said appending in (d) is performed by ligation. In some embodiments, said appending in (h) is performed by PCR by using primers that contain said second sequencing adapter and a target-specific sequence downstream of said elongation sequence. In some embodiments, the method further comprises fragmenting said barcode-tagged and elongated nucleic acid molecules prior to said appending in (h).

In some embodiments, said appending in (a) takes place inside a partition. In some embodiments, said appending in (a) is performed by primer extension. In some embodiments, said appending in (a) is performed by reverse transcription. In some embodiments, said appending in (a) is performed by ligation. In some embodiments, different elongation sequences are appended to different copies of said nucleic acid molecules sharing the same molecule-specific barcode, thereby generating a pool of barcode-tagged nucleic acid molecules with different elongation sequences complementary to different internal positions. In some embodiments, said different internal positions cover the length of said nucleic acid molecule or discontiguous regions of interest by design. In some embodiments, said elongation sequence comprises a random sequence of a length of at least 6 bases. In some embodiments, said elongation sequence comprises a random sequence of a length of at least 8 bases. In some embodiments, said elongation sequence comprises a random sequence of a length of at least 10 bases. In some embodiments, said elongation sequence comprises a random sequence of a length of at least 12 bases. In some embodiments, said elongation sequence comprises a random sequence of a length of at least 16 bases. In some embodiments, said elongation sequence comprises a random sequence of a length of at least 20 bases. In some embodiments, said denaturing is performed by heat denaturation under dilute condition. In some embodiments, said denaturing is performed by alkaline denaturation under dilute condition. In some embodiments, said denaturing is performed by 5′ phosphorylation of a strand to be removed and enzymatic digestion by lambda exonuclease. In some embodiments, said denaturing is performed by appending a strand to be removed with 5′ biotinylation, immobilizing said strand on streptavidin-coated solid-surface, and releasing said strand for elongation through washing and/or denaturation. In some embodiments, said extending is performed isothermally. In some embodiments, said extending is performed by primer annealing at one temperature and extension at a different temperature.

In some embodiments, the nucleic acid sequence is obtained for a nucleic acid sequence comprising a length of at least about 500 bases. In some embodiments, the nucleic acid sequence is obtained for a longer nucleic acid sequence comprising a length of at least about 1000 bases. In some embodiments, the nucleic acid sequence is obtained for a longer nucleic acid sequence comprising a length of at least about 1000 or more bases. In some embodiments, the nucleic acid sequence is obtained for a longer nucleic acid sequence comprising a length of at least 1 kilobase to about 20 kilobases.

### INCORPORATION BY REFERENCE

All publications, patents, patent applications, and NCBI accession numbers mentioned in this specification are herein incorporated by reference to the same extent as if each individual publication, patent, patent application, or NCBI accession number was specifically and individually indicated to be incorporated by reference, unless only specific sections of patents, patent applications, or publications are indicated to be incorporated by reference. To the extent publications, patents, patent applications, or NCBI accession numbers incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an overview of an illustrative method for obtaining assembled single-molecule synthetic long read from nucleic acid molecules inside single cells using intramolecular ligation.

FIG. 2 depicts an overview of an illustrative method for obtaining assembled single-molecule synthetic long read from nucleic acid molecules inside single cells using intramolecular elongation.

FIG. 3 depicts the structure of illustrative Terminal Tags and Template-Switching Oligonucleotides with partition-specific and molecule-specific barcodes.

FIG. 4 depicts an exemplary illustration of single cell encapsulation with barcoded microparticles.

FIG. 5 and FIG. 6 depict exemplary illustrations of tagging single molecules and distributing barcodes to locations within the target molecules for generating short nucleic acid molecules.

FIG. 7 depicts an exemplary illustration of an alternative method for tagging single molecules and distributing barcodes to locations within the target molecules for generating short nucleic acid molecules.

FIG. 8 depicts position mapping of example short reads from a unique molecular barcode. Short reads with molecular barcode sequence GCTTCCTTCTGA (SEQ ID NO: 1) were mapped to the reference sequence NM_001323960.1 (SEQ ID NO: 30). Short reads map only to the 3′ end of the RNA transcript with existing 3′ RNAseq technology. Short reads map to the 3′ end of the RNA transcript as well as throughout the length of the transcript with the synthetic long read technology of the present disclosure.

FIG. 9 depicts position mapping of example short reads from a unique molecular barcode. Short reads with molecular barcode sequence GTCAGAAGCACT (SEQ ID NO: 2) were mapped to the reference sequence NM_001688.4 (SEQ ID NO: 31). Short reads map only to the 3′ end of the RNA transcript with existing 3′ RNAseq technology. Short reads map to the 3′ end of the RNA transcript as well as throughout the length of the transcript with the synthetic long read technology of the present disclosure.

### DETAILED DESCRIPTION

While some embodiments of the present disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

Unless defined otherwise, all technical and scientific terms used herein have the same meaning as commonly understood by one of ordinary skill in the art to which the present disclosure belongs. In case of conflict, the present disclosure including the definitions will control. Also, unless otherwise required by context, singular terms shall include pluralities and plural terms shall include the singular.

Currently, the length of cDNA sequences that can be read, for example using 3′ and 5′ tagging and sequencing of mRNA molecules from single cells, can be limited to the sequencing length of massively parallel sequencing technology, i.e. the read-length of short-read sequencing technologies. The read-length using these short-read sequencing technologies can be in the range of 100-500 base pairs (bp). However, when the gene of interest or region of interest is longer than the read-length, and/or when the region of interest is not within the read-length of the 3′ or the 5′ end of the molecule, sequence information of the mRNA molecule can be lost. In addition, mRNA molecules can undergo splicing from precursor mRNA transcribed from DNA to remove the introns and ligate the exons together, often in a combinatorial manner. Different mRNA variants, known as splicing variants, can arise from alternative splicing of the same nascent precursor messenger RNAs. These splicing variants can share the same 3′ and/or 5′ sequence but not the intervening sequence in the mature mRNA form. Consequently, obtaining only the 3′ or the 5′ sequence of the mRNA molecules can mask the real sequence of mRNA molecules and hence the true diversity of the transcriptome, potentially obscuring single-cell differential gene expression analysis.

One possible method to circumvent the read-length problem is synthetic long read (SLR) sequencing, in which one can tag the same nucleic acid molecule several times with the same partition-specific barcode, each barcode copy tagging at a different location along the nucleic acid molecule, either through random fragmentation and ligation of the partition specific tag or appending the partition specific tag through random oligonucleotide priming. The short-read sequence information resulting from nucleic acid libraries prepared in this manner can then be used to reconstruct the sequence of the original nucleic acid molecules by assembling the overlapping short reads from each partition into distinct sequences of nucleic acid molecules. A drawback of this approach can be that this method may not be able to differentiate between nucleic acid molecules that have significant stretches of sequence that are identical or very similar compared to other molecules in the same cell/partition. For example, in the case of mRNA splice variants, the assembly-by-homology approach may not be able to determine whether certain short-read sequences originate from the same mRNA molecule or from a different mRNA splice variant of the same gene within the same cell. The same can be true for homologous stretches of genomic DNA within the same cell. This inability to accurately cluster and assemble short sequencing reads by their molecular origin can be known as the phasing problem.

To solve the phasing problem, short read data can be used to deduce long read sequencing information. Nucleic acid molecules (e.g., several kilobases in length) from a single cell can be diluted into many partitions such that each partition has a low probability of containing molecules of high homology. The nucleic acid content in each partition can be tagged with partition-specific barcodes, amplified, and converted into short-read sequencing libraries. The partition-specific barcodes can be used to assemble the short-read sequence information back to the original long molecule. However, dilution-based SLR approaches may fail when there exist many highly homologous molecules in the sample, such that the molecules inside each partition are not unique. In this scenario, the partition-specific barcodes may not be able to differentiate homologous molecules from each other, since the assembly of short-read sequence information relies on the use of homology between short-reads and the assumption that sequences that share homology come from the same starting molecule. As such, existing SLR approaches may not accurately phase high homology sequences since they cannot determine whether specific short-read sequencing data originates from a particular nucleic acid molecule or from a similar/homologous molecule, hence failing to generate synthetic long reads from short read information.

Without a way to differentiate homologous molecules inside each partition from each other, current SLR technologies fall short of being able to solve the phasing problem for single cell sequencing. Thus, there remains an unmet need for SLR methods that can facilitate single cell phased sequencing of a nucleic acid molecule, including molecules with homology to other molecules within the same cell. A method of the present disclosure can meet that need by providing a method that can clonally distribute molecule-specific barcodes to various locations along long nucleic acid molecules, addressing the aforementioned single cell phasing problem by ensuring that short-read sequencing information spanning the entire length of nucleic acid molecules can be traced back to both its cell/partition and to its single molecule origin. The present disclosure can increase the read length of single cell sequencing from the nucleic acid termini to the entire length of the molecule or to specific regions of the molecule, and can reduce coverage bias of the long molecules.

Thus, the present disclosure can relate to a method for tagging single nucleic acid molecules for single-cell synthetic long-read (SLR) DNA sequencing or RNA sequencing. For example, the method can comprise encapsulating single cells into individual partitions and/or extracting its nucleic acid content inside each partition. The method can include tagging the nucleic acid molecules inside each partition with terminal adapters comprising partition-specific barcodes and/or unique molecule-specific barcodes, thereby obtaining a pool of uniquely barcoded DNA molecules that share the same partition-specific barcode inside each partition. The method can also provide a plurality of clonal nucleic acid molecules, and each nucleic acid molecule can have the same partition-specific and molecule-specific barcodes at the terminal ends. Alternatively, each nucleic acid molecule can have different partition-specific and molecule-specific barcodes at the terminal ends. The method can further comprise fragmenting the nucleic acid at a random location inside the molecule. The nucleic acid molecule can be barcoded and/or for each copy of the barcoded nucleic acid molecule, the terminal barcoded end can be joined with the end generated by random fragmentation. For example, the method can comprise circularizing the molecule via intramolecular ligation. The method can also comprise sequencing the partition-specific barcode, the molecule-specific barcode, and the internal sequence of the molecule up to and including the end generated by random fragmentation. After sequencing, the method can comprise clustering the sequencing data by the molecule-specific barcodes and assembling synthetic long read sequencing data from each barcode cluster for each molecule from the plurality of shorter internal sequences of the nucleic acid molecule. Clustering the synthetic long-read sequencing data by the cell-specific barcodes can generate cell-specific long-read sequencing data. Data generated by the methods described herein can allow differentiating between distinct phases, including molecular variants of highly homologous molecules.

The present disclosure can relate to a method for tagging single nucleic acid molecules for single-cell synthetic long-read (SLR) DNA sequencing or RNA sequencing. The method can comprise encapsulating single cells into individual partitions and extracting the nucleic acid content inside each partition. The method can comprise tagging the nucleic acid molecules inside each partition with partition-specific barcodes on one terminal end and/or tagging the nucleic acid molecules with unique molecule-specific barcodes on the opposing terminal end, thereby obtaining a pool of uniquely barcoded DNA molecules. The method can also provide a plurality of clonal nucleic acid molecules each having the same partition-specific and molecule-specific barcodes at the terminal ends. The method can further comprise fragmenting the nucleic acid at a random location inside the molecule. The method can comprise for example, circularizing the molecule via intramolecular ligation in order to join the terminal end of nucleic acid molecules with molecule-specific barcodes and the end generated by random fragmentation. Sequencing of the partition-specific barcode can follow. For example, sequencing can include the sequencing of the molecule-specific barcode and the internal sequence of the molecule up to and including the end generated by random fragmentation. The method can further comprise assembling the sequence of the nucleic acid molecule from the plurality of internal sequences. Data generated by the methods described herein can allow differentiating between distinct phases, including molecular variants of highly homologous molecules.

The present disclosure can provide a method for tagging single nucleic acid molecules for single-cell synthetic long-read (SLR) DNA sequencing or RNA sequencing. The method can comprise encapsulating single cells into individual partitions and extracting its nucleic acid content inside each partition. Tagging of the nucleic acid molecules can occur inside each partition with partition-specific barcodes on one terminal end and/or with unique molecule-specific barcodes on the opposing terminal end. Thus, generating a pool of uniquely barcoded DNA molecules. The method can further provide a plurality of clonal nucleic acid molecules, in which each can have the same partition-specific and molecule-specific barcodes at the terminal ends. The terminal end with the partition-specific barcode can be joined with the terminal end with the molecule-specific barcode. Circularization of the molecule can be performed via intramolecular ligation. The method can further comprise sequencing the partition-specific barcode and the molecule-specific barcode, pairing the molecule-specific barcode with the partition-specific barcode from the plurality of barcode sequences, and differentiating between the sequences of nucleic acid molecules from different partitions.

The present disclosure can provide a method for tagging single nucleic acid molecules for single-cell synthetic long-read (SLR) DNA sequencing or RNA sequencing. The method can comprise encapsulating single cells into individual partitions and extracting its nucleic acid content inside each partition, and tagging the nucleic acid molecules inside each partition with terminal adapters comprising partition-specific barcodes and unique molecule-specific barcodes, thereby obtaining a pool of uniquely barcoded DNA molecules. The method can provide a plurality of clonal nucleic acid molecules each having the same partition-specific and molecule-specific barcodes at the terminal ends. The terminal end containing barcodes can append with an elongation sequence that is also internal to the long nucleic acid molecule. Denaturing and obtaining single-stranded DNAs with the elongation sequence on the 3′ terminal end for intramolecular priming can follow. The method can comprise annealing the 3′ terminal end with the elongation sequence at an internal position intramolecularly, extending the molecule, and sequencing the partition-specific barcode, the molecule-specific barcode, and the internal sequences downstream of the elongation sequence. The method can comprise assembling the sequence of the nucleic acid molecule from the plurality of internal sequences of the nucleic acid molecule and differentiating between distinct phases. Data generated by the methods described herein can allow differentiating between distinct phases, including molecular variants of highly homologous molecules.

The present disclosure can provide a method for tagging single nucleic acid molecules for single-cell synthetic long-read (SLR) DNA sequencing or RNA sequencing. The method can comprise encapsulating single cells into individual partitions and extracting its nucleic acid content inside each partition. The method can comprise tagging the nucleic acid molecules inside each partition with partition-specific barcodes on one terminal end, and tagging the nucleic acid molecules with unique molecule-specific barcodes on the opposing terminal end, thereby obtaining a pool of uniquely barcoded DNA molecules. The method can provide a plurality of clonal nucleic acid molecules each having the same partition-specific and molecule-specific barcodes at the terminal ends. The method can comprise appending the terminal end containing the molecule-specific barcodes with an elongation sequence that is also internal to the long nucleic acid molecule. Denaturing and obtaining single-stranded DNAs with the elongation sequence on the 3′ terminal end for intramolecular priming can follow. The method can further comprise annealing the 3′ terminal end with the elongation sequence at an internal position intramolecularly and extending the molecule, and sequencing the partition-specific barcode, the molecule-specific barcode, and the internal sequences downstream of the elongation sequence. The method can comprise assembling the sequence of the nucleic acid molecule from the plurality of internal sequences of the nucleic acid molecule. Data generated by the methods described herein can allow differentiating between distinct phases, including molecular variants of highly homologous molecules.

The present disclosure can provide a method of obtaining nucleic acid sequence information from a nucleic acid molecule by assembling a plurality of short nucleic acid sequences into a longer nucleic acid sequence. The method can comprise attaching a terminal tag comprising a sequencing adapter sequence, a universal PCR sequence, a partition-specific barcode, and a molecule-specific barcode, with or without a target molecule sequence to one end of a plurality of nucleic acid molecules to form a pool of barcode-tagged molecules. A second terminal tag can be attached on the opposing end of the barcode tag, comprising a universal PCR sequence, with or without a target molecule sequence. The method can comprise amplifying the barcode-tagged molecules to obtain a library of barcode-tagged molecules with many copies of identical molecules and fragmenting the barcode-tagged molecules, thereby generating barcode-tagged fragments comprising of the barcode sequence on one end and an unknown sequence from an internal region on the other end. The method can comprise circularizing the barcode-tagged fragments comprising of the barcode sequence on one end and an unknown sequence from an internal region on the other end via intramolecular ligation, thereby bringing the barcode sequence into proximity with the unknown sequence from an internal region. Fragmenting the circularized barcode-tagged fragments into linear, barcode-tagged molecule, with the barcode sequence at the internal region of the linear molecule can be performed. A second sequencing adapter can attach to each end of the linear barcoded-fragment to form double adapter-ligated barcode-tagged nucleic acid fragments. The method can further comprise amplifying all or part of the double adapter-ligated barcode-tagged nucleic acid fragments, and sequencing the double adapter-ligated barcode-tagged nucleic acid fragments. The method can also comprise clustering the sequenced nuclear acid fragments into groups using the molecule-specific barcodes and assembling each group of reads with the same molecule-specific barcodes into long nucleic acid sequence.

The present disclosure can provide a method of obtaining nucleic acid sequence information from a nucleic acid molecule by assembling a plurality of short nucleic acid sequences into a longer nucleic acid sequence. The method can comprise attaching a terminal tag comprising a universal PCR sequence and a partition-specific barcode, with or without a target molecule sequence to one end of a plurality of nucleic acid molecules to form a pool of barcode-tagged molecules. A second terminal tag can then be attached on the opposing end of the first barcode tag, comprising a sequencing adapter sequence, a universal PCR sequence, and a molecule-specific barcode, with or without a target molecule sequence. The barcode-tagged molecules can be amplified to obtain a library of barcode-tagged molecules with many copies of identical molecules. The method can comprise fragmenting the barcode-tagged molecules, thereby generating barcode-tagged fragments comprising of the barcode sequence on one end and an unknown sequence from an internal region on the other end. The method can comprise circularizing the barcode-tagged fragments comprising of the barcode sequence on one end and an unknown sequence from an internal region on the other end via intramolecular ligation, thereby bringing the barcode sequence into proximity with the unknown sequence from an internal region. The method can further comprise fragmenting the circularized, barcode-tagged fragments into linear, barcode-tagged molecule, with the barcode sequence at the internal region of the linear molecule. A second sequencing adapter can then attach to each end of the linear barcoded-fragment to form double adapter-ligated barcode-tagged nucleic acid fragments. All or part of the double adapter-ligated barcode-tagged nucleic acid fragments can be amplified. Sequencing of the double adapter-ligated barcode-tagged nucleic acid fragments can follow. The method can further comprise clustering the sequenced nuclear acid fragments into groups using the molecule-specific barcodes and assembling each group of reads with the same molecule-specific barcodes into long nucleic acid sequence.

The present disclosure can provide a method of obtaining nucleic acid sequence information from a nucleic acid molecule by assembling a plurality of short nucleic acid sequences into a longer nucleic acid sequence. The method can comprise attaching a terminal tag comprising a sequencing adapter sequence, a universal PCR sequence, a partition-specific barcode, and a molecule-specific barcode, with or without a target molecule sequence to one end of a plurality of nucleic acid molecules to form a pool of barcode-tagged molecules. A second terminal tag can be attached on the opposing end of the barcode tag, comprising a universal PCR sequence, with or without a target molecule sequence. The method can further comprise amplifying the barcode-tagged molecules to obtain a library of barcode-tagged molecules with many copies of identical molecules and appending the terminal end containing the barcodes with an elongation sequence that is also internal to the long nucleic acid molecule. Denaturing or removing one of the two strands of the double-stranded barcoded-tagged molecule with elongation sequence is then performed, thereby generating barcode-tagged molecules comprising of the barcode sequence and an elongation sequence on the 3′ end. The 3′ terminal end can be annealed with the elongation sequence at an internal position intramolecularly to extend the molecule, thereby bringing the barcode sequence into proximity with the internal region that is complementary to the elongation sequence. A second sequencing adapter can attach to the intramolecularly elongated barcoded molecule to form double-adapter barcode-tagged nucleic acid fragments. The method can further comprise amplifying all or part of the double-adapter barcode-tagged nucleic acid fragments and sequencing the double-adapter barcode-tagged nucleic acid fragments. The method can also comprise clustering the sequenced nucleic acid fragments into groups using the molecule-specific barcodes and assembling each group of reads with the same molecule-specific barcodes into long nucleic acid sequence.

The present disclosure can provide a method of obtaining nucleic acid sequence information from a nucleic acid molecule by assembling a plurality of short nucleic acid sequences into a longer nucleic acid sequence. The method can comprise attaching a terminal tag comprising a universal PCR sequence, and a partition-specific barcode, with or without a target molecule sequence to one end of a plurality of nucleic acid molecules to form a pool of barcode-tagged molecules. The method can further comprise attaching a second terminal tag on the opposing end of the partition-specific barcode tag, comprising a sequencing adapter sequence, a universal PCR sequence, and a molecule-specific barcode, with or without a target molecule sequence. The method can comprise amplifying the barcode-tagged molecules to obtain a library of barcode-tagged molecules with many copies of identical molecules, and appending the terminal end containing barcodes with an elongation sequence that is also internal to the long nucleic acid molecule. Denaturing or removing one of the two strands of the double-stranded barcoded-tagged molecule with elongation sequence can then follow, thereby generating barcode-tagged molecules comprising of the barcode sequence and an elongation sequence on the 3′ end. The method can comprise annealing the 3′ terminal end with the elongation sequence at an internal position intramolecularly and extending the molecule, thereby bringing the barcode sequence into proximity with the internal region that is complementary to the elongation sequence. A second sequencing adapter can attach to the intramolecularly elongated barcoded molecule to form double-adapter barcode-tagged nucleic acid fragments. Amplification of all or part of the double-adapter barcode-tagged nucleic acid fragments, and sequencing of the double-adapter barcode-tagged nucleic acid fragments can be performed. The method can further comprise clustering the sequenced nucleic acid fragments into groups using the molecule-specific barcodes and assembling each group of reads with the same molecule-specific barcodes into long nucleic acid sequence.

The present disclosure can provide a method for obtaining long-read, single-cell nucleic acid information constructed from short nucleic acid sequences. Sequencing of target nucleic acid molecules that are longer than the read-length of current short-read sequencers can be accomplished using the methods of the present disclosure by for example, assembling intermediate and long nucleic acid sequences from short nucleic acid sequences. The method of the present disclosure can be more accurate than other methods for obtaining nucleic acid sequence information by clustering overlapping short-reads and correcting for errors that may have been introduced during NGS sample preparation and during short-read sequencing.

The method can be useful in haplotyping by allowing for the identification and differentiation of variations on the same or different chromosomes that are otherwise bracketed by regions of homology. Phasing information, i.e. the connectivity between variants, can be provided using the methods of the present disclosure because the methods allow association of variants that are separated by a distance greater than the read-length of a current short-read sequencer. The phased sequence can be utilized for determining expression of previously unidentified alternative transcripts, for quality control of synthesized long DNA molecules, for identifying the length of repetitive sequences and the like. The present disclosure can provide a means for obtaining high-quality, long phased DNA sequences.

Partitioning single cells into individual physical partitions can be used to characterize the cells nucleic acid molecules individually. In addition, nucleic acid molecules of single cells can be decoupled from nucleic acid molecules of ensembled cells when characterized in bulk. Tagging long nucleic acid molecules with barcodes and obtaining short nucleic acid sequencing information from the long nucleic acid molecules can be performed using the methods of the present disclosure. The sequencing information from the long nucleic acid molecules can be obtained by assembling a series of short nucleic acid sequences into longer nucleic acid sequences. The barcodes that can tag the long nucleic acid molecules can be used to identify the origin of the nucleic acid sequencing information. This can include for example, the physical partitions that the long nucleic acid molecules can be extracted from, and the long nucleic acid molecules that the short sequencing information is obtained from.

Barcode tagging of nucleic acid contents can be performed in a sequence dependent manner or a sequence independent manner. Sequence dependent barcode tagging can be performed by utilizing sequence specific or partial sequence specific primers during barcode tagging. As a non-limiting example, when investigating alternatively spliced transcripts, the barcode can be added specifically to the sequences of interests using a forward primer complementary to exon 1 of the transcript, which most often is known, and a reverse primer complementary to the poly-A tail terminating all alternatively spliced transcripts. A unique barcode sequence can be added at the 3′ end of each primer in the primer mixture, such that the product obtained include all alternative transcripts initiated from the specific exon 1, wherein each amplicon is flanked by a unique barcode sequence at both ends thereof. In some cases, only the forward primer includes a barcode sequence, thereby obtaining PCR products having a unique barcode sequence at the 5′ end only.

Sequence independent barcode tagging can be performed by utilizing primers that can comprise a common sequence that is independent of the internal sequence of interest. As a non-limiting example, when investigating whole-cell mRNA sequences, the barcode can be added to all the mRNA molecules by utilizing a reverse transcription primer complementary to the poly-A tail shared by all mRNA transcripts. The reverse transcription can be conducted with a reverse transcriptase with a terminal transferase and strand-switching activity. The short cytosine repeats that are appended by the reverse transcriptase when it reaches the 5′ end of the mRNA transcripts can be used to attach the barcode sequence. Sequence-independent barcode tagging can be performed by utilizing primers comprising a random sequence that can prime at unknown locations in a pool of target nucleic acid molecules. Alternatively, sequence-independent barcode tagging can be performed by directly attaching the barcodes at the terminal ends of the target nucleic acid molecules via ligation.

Barcode tagging of target nucleic acid molecules can include tagging the molecules with partition-specific barcodes, where a plurality of molecules inside each partition share the same partition-specific barcodes, as well as tagging the molecules with molecule-specific barcodes, where each molecule inside each partition has a unique molecule-specific barcode. The nucleic acid molecules can be tagged at their 5′ end and/or 3′ end with both partition-specific barcodes and the molecule-specific barcodes or one barcode at each end, e.g., a partition-specific barcode at the 5′ end and a molecule-specific barcode at the 3′ end, or vice versa. This can be done for example by primer extension using oligonucleotides comprising the barcodes, reverse transcription using oligonucleotides comprising the barcodes, or blunt end ligation between the nucleic acid molecules and ligation adapters comprising the barcodes.

The method can comprise generating for each long nucleic acid molecule in mixture, e.g. nucleic acid molecules extracted from a single cell inside a physical partition, a pool of short nucleic acid molecules that have the same barcode, which is unique to each long nucleic acid molecule. The short nucleic acid molecules can cover the entire length of the long molecules or cover specific regions of interest within the long molecules. The specific regions of interest can be discontiguous, e.g., separated by regions of homology or regions that are otherwise not the focus of the sequencing effort and consequently omitted in the sequencing information collection.

The method can further comprise fragmenting the pool of nucleic acid molecules into a plurality of shorter nucleic acid molecules that are still longer than the read length of short-read sequencer inside the physical partitions. Fragmentation of the nucleic acid molecules can be necessary when the pool of nucleic acid molecule is genomic DNA. The nucleic acid molecules can be amplified, in a sequence dependent or sequence independent manner, prior to fragmentation inside the physical partitions.

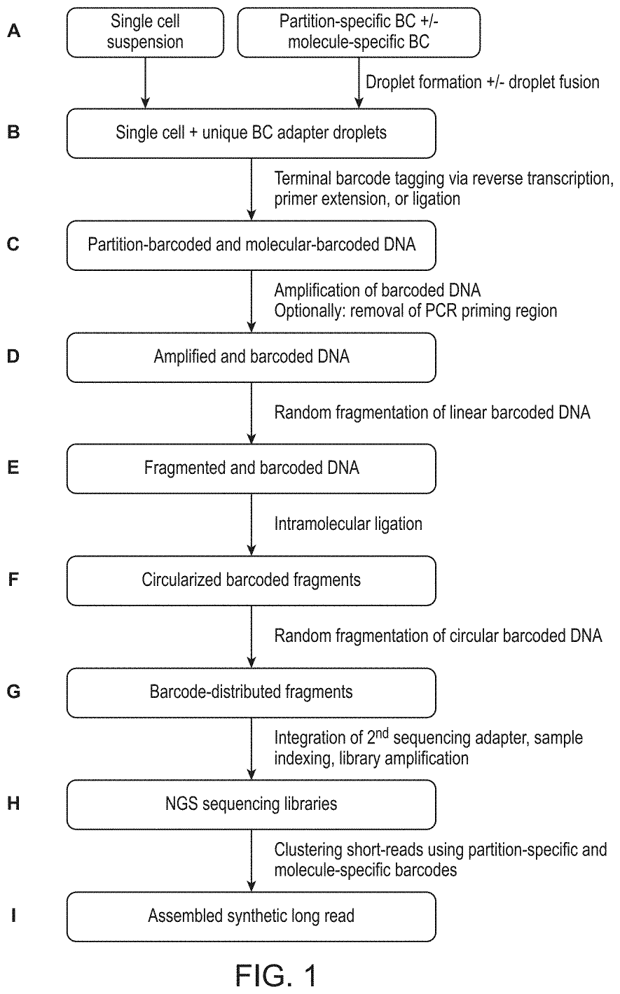

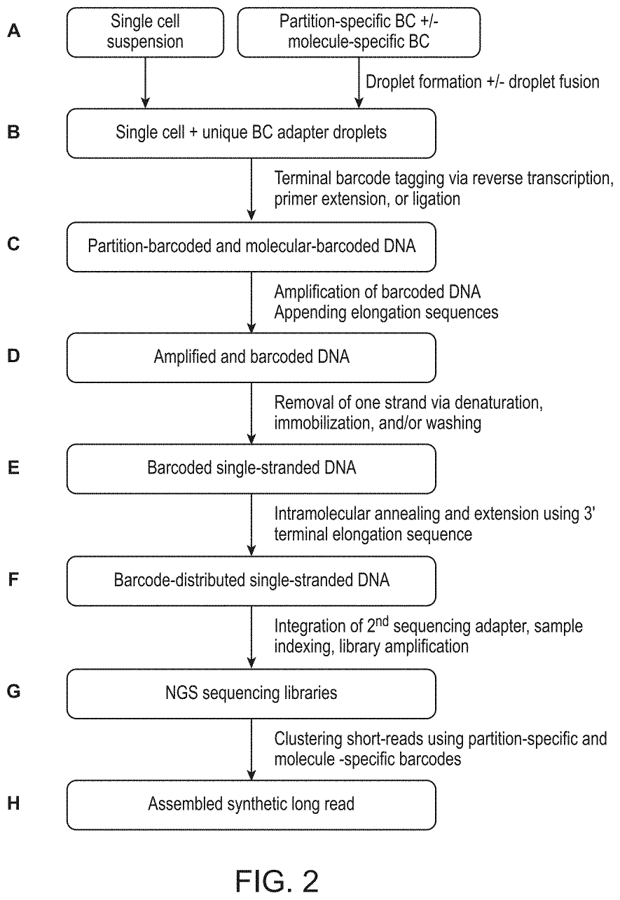

Exemplary workflow overviews of the present disclosure are illustrated in FIG. 1 and FIG. 2. A plurality of nucleic acid molecules can be tagged with partition-specific and molecule specific barcodes (FIG. 1 C). The tagged plurality of nucleic acid molecules, each having the same partition-specific and molecule-specific barcode, can be amplified (FIG. 1 D) to create many copies of each barcoded nucleic acid molecule. This can facilitate downstream processing, wherein short nucleic acid molecules collectively cover the long molecules or specific regions of the long molecule. The short nucleic acid molecules can be assembled into one or more long nucleic acid sequences, said method comprising: fragmenting the barcode-tagged nucleic acid molecules at an unknown location internal to the long nucleic acid molecules, each clonal copy of the long nucleic acid molecule fragmented at a different unknown location (FIG. 1 E); circularizing the fragmented barcode-tagged nucleic acid molecules, thereby distributing and proximating the barcodes to different locations within the target nucleic acid molecules (FIG. 1 F); fragmenting the circularized, barcode-tagged nucleic acid fragments; attaching a second sequencing adapter to the linear barcode-tagged nucleic acid fragments (FIG. 1 G); amplifying the sequences bracketed by the sequencing adapter, including the barcodes and the internal sequence of the long nucleic acid molecules (FIG. 1 G); sequencing the double-adapter barcode tagged short nucleic acid molecules (FIG. 1H); clustering the short nucleic acid molecules using the partition-specific and molecule-specific barcodes (FIG. 1 H); and assembling each cluster of short nucleic acid sequence into one or more long nucleic acid sequences (FIG. 1 I).

The method can further comprise removing the PCR primer region from the barcode-tagged sequences. For example, removing the PCR primer region can be carried out prior to circularizing the barcode-tagged fragments. Alternatively, removing the PCR primer region can be carried out prior to fragmenting the barcode-tagged molecules at unknown locations.

While generating a plurality of clonal nucleic acid molecules, different elongation sequences can be appended to nucleic acid molecules, such that different nucleic acid molecules that originate from the same long nucleic acid molecule can have the same partition-specific and molecule-specific barcode but different elongation sequence (FIG. 2 D). The elongation sequence can be complementary to an internal sequence of the target nucleic acid molecule or can comprise a random sequence. This can facilitate downstream processing, wherein short nucleic acid molecules collectively cover the long molecules or specific regions of the long molecule. The short nucleic acid molecules can be assembled into one or more long nucleic acid sequences, said method comprising: generating single-stranded barcode-tagged nucleic acid molecule with the elongation sequence at the 3′ end (FIG. 2 E); annealing the 3′ terminal end with the elongation sequence of the barcode-tagged nucleic acid molecules at an internal position intramolecularly (FIG. 2 F); extending the intramolecularly annealed 3′ end at either known internal locations or unknown locations depending on the nature of the elongation sequence, thereby distributing and proximating the barcodes to different locations within the target nucleic acid molecules (FIG. 2 F); attaching a second sequencing adapter to the elongated barcode-tagged nucleic acid molecule (FIG. 2 G); amplifying the sequences bracketed by the sequencing adapter, including the barcodes and the internal sequence of the long nucleic acid molecules (FIG. 2 G); sequencing the double-adapter barcode tagged short nucleic acid molecules; clustering the short nucleic acid molecules using the partition-specific and molecule-specific barcodes; and assembling each cluster of short nucleic acid sequence into one or more long nucleic acid sequences.

Standard NGS library preparation can be utilized to convert barcode-tagged and barcode-distributed nucleic acid molecules to NGS libraries for short-read sequencing. The method can comprise: fragmenting the barcode-distributed nucleic acid molecules at random locations with lengths suitable for short-read sequencing; blunting the terminal ends by truncating the 3′ protruding ends and filling in the 3′ recessed ends; a-tailing the blunted terminal ends; ligating a second sequencing adapter via TA ligation; and amplifying the double-adapter short nucleic acid molecules.

NGS library preparation using PCR amplification can be utilized to convert the barcode-distributed nucleic acid molecules to NGS libraries for short-read sequencing. The method can comprise: priming and amplification of the barcode-distributed nucleic acid molecules using a primer comprising the same sequencing adapter that is incorporated during nucleic acid molecule tagging and a second sequencing adapter and gene-specific sequences that can be internal to the target nucleic acid molecule; and further amplifying the double-adapter short nucleic acid molecules.

Sequence information from uniquely barcoded nucleic acid molecules can be obtained after NGS library preparation and short-read sequencing. The method can further comprise phasing the obtained sequences based on their molecular origin as indicated by the unique partition-specific and molecule-specific barcode. The short-read sequencing information can be clustered using the partition-specific followed by the molecule-specific tags and assembled into de novo sequences. The resulting sequences can be phased reconstruction of the original long nucleic acid molecules and can share any degree of homology or similarity with each other. By comparing long sequences that are identical or share any commonality in their classification with each other, the present method can provide a distinct advantage in quantitative analysis for estimating the abundance of different molecules in a pool of parental long molecules.

The present disclosure can provide systems and methods for preparing nucleic acids for high-throughput single-cell long-read sequencing, including high-throughput, scalable partitioning of single cells, efficient tagging, and sequencing complex nucleic acid content inside each cell. In addition, the present disclosure can facilitate phased, long-read sequence information to be inferred from the short-read sequencing of nucleic acid molecules.

It is understood that the present disclosure is not limited to the particular methodology, protocols, and reagents, etc., described herein, as these can be varied by the skilled artisan. It is also understood that the terminology used herein is used for the purpose of describing particular illustrative embodiments only, and is not intended to limit the scope of the disclosure. As used herein and in the specification appended claims, the singular forms “a”, “an”, and “the” include the plural referents unless the context clearly dictates otherwise. Thus, for example, a reference to “a DNA molecule” is a reference to one or more DNA molecules and equivalents thereof, a “polynucleotide” includes a single polynucleotide as well as two or more of the same or different polynucleotides, and reference to an “nucleic acid” includes a single nucleic acid as well as two or more of the same or different nucleic acids, and the like.

The embodiments of the present disclosure and the various features and advantageous details thereof are explained more fully with reference to the non-limiting embodiments and examples that are described and/or illustrated in the accompanying drawings and detailed in the following description. It should be noted that features of one embodiment may be employed with other embodiments as the skilled artisan would recognize, even if not explicitly stated herein. Descriptions of well-known components and processing techniques may be omitted so as to not unnecessarily obscure the embodiments of the disclosure.

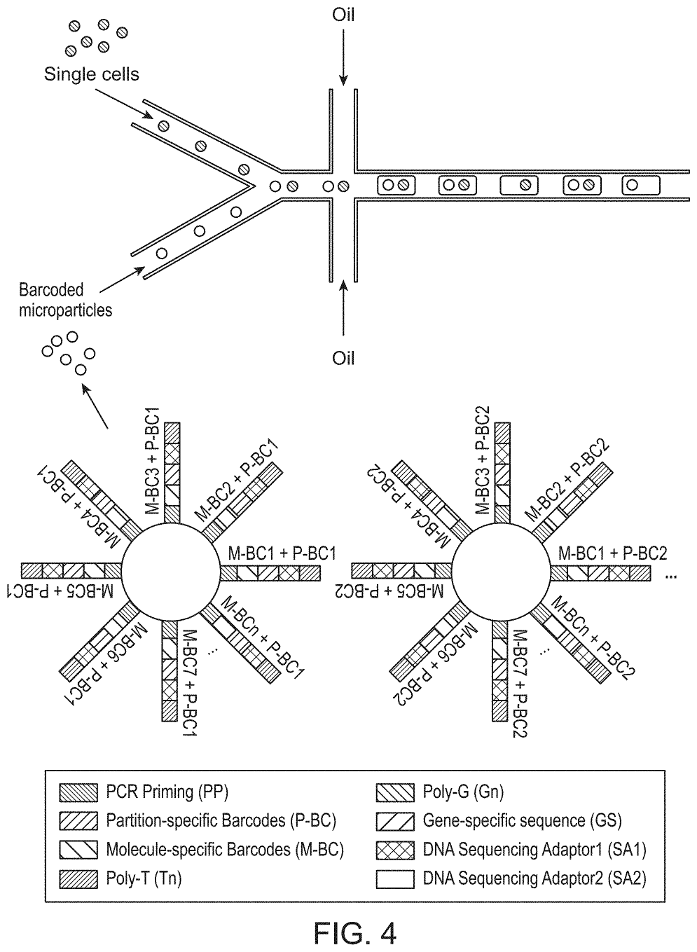

The present disclosure can provide a method for encapsulating single cells into individual partitions, lysing the cells inside the partition, and tagging long DNA or RNA molecules for synthetic long-read (SLR) sequencing. The method can provide for single cells in a sample to be partitioned inside an aqueous droplet with lysis reagent and a microparticle that has been functionalized to contain many copies of a partition-specific tag that is unique to the population of all the microparticles used (FIG. 4). The method can provide for each long nucleic acid molecule in the lysed cellular mixture to be tagged with a molecule-specific barcode that is unique inside each partition. The method can also provide for each long nucleic acid molecule in a cellular mixture to generate a pool of short DNA molecules that have the same molecule-specific barcodes that are unique inside each partition, such that the short DNA molecules collectively span and cover the entire length of the long molecules or cover specific regions of interest by design.

A single-cell suspension can be partitioned into aqueous droplets and co-encapsulated with a barcoded microparticle by co-flowing the single-cell suspension in one channel and a microparticle suspended in lysis buffer in another channel across an oil channel. By controlling the flow rates of the two aqueous channels and the oil channel, a specific size of the aqueous droplet and a specific rate of droplet generation can be achieved. By controlling the concentration of the single-cell suspension and the microparticle suspension, aqueous partitions that can contain either one or no cell and either one or no barcoded microparticles can be achieved. Since the partition-specific tag and/or molecule-specific tag can also contain a universal sequencing adapter that is used to enrich for the correctly tagged long molecules, single-cell droplets without the partition-specific tags and/or molecule-specific tag are generally not included in the final sequencing library.

A single-cell suspension can be partitioned into aqueous droplets without a barcoded microparticles. By controlling the concentration of the single-cell suspension, aqueous partitions that can contain either one or no cell can be achieved. In addition, lysis buffer and solutions of oligonucleotides containing partition-specific barcodes can also be used to generate aqueous droplets, such that each droplet can contain many copies of single partition-specific barcodes. Once aqueous droplets containing single cells and single sequence of partition-specific barcode per partition can be obtained, they can be co-flowed and fused with each other. Since the partition-specific tag and/or molecule-specific tag can also contain a universal sequencing adapter, only the correctly and doubly tagged long molecules are enriched and included in the final sequencing library.

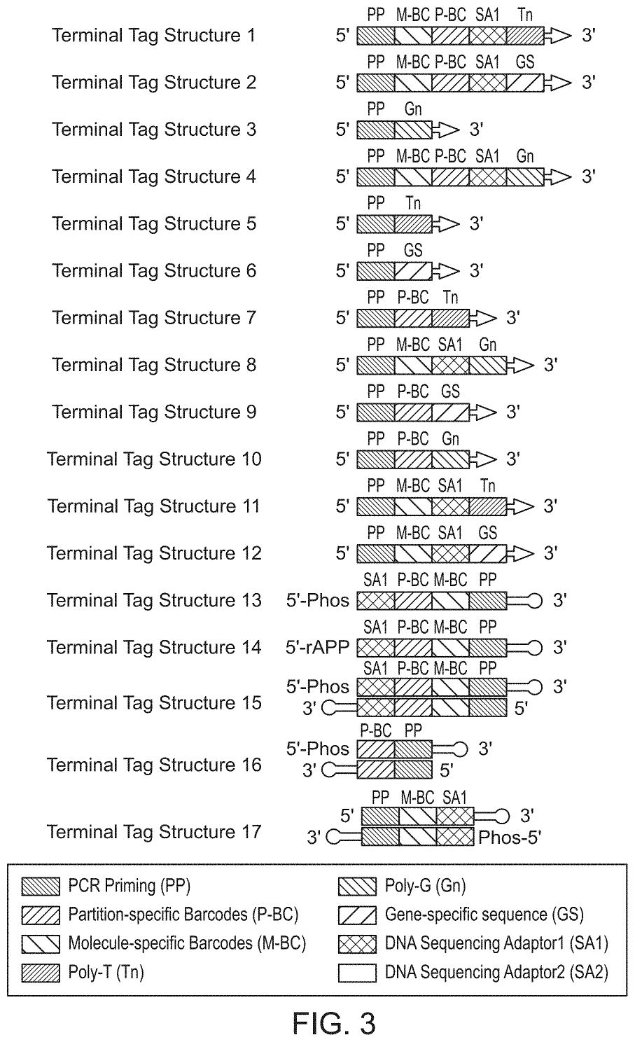

The targets for SLR sequencing can be RNA molecules. The terminal tags that are unique to each partition can comprise of a sequencing adapter, a universal PCR sequence, a partition-specific barcode, a molecule-specific barcode, and/or a poly-thymine sequence. FIG. 3 Terminal Tag Structure 1 is an exemplary adapter that can be useful in the present disclosure. The RNA molecules inside each partition can be tagged during reverse transcription using the poly-thymine sequence as the priming site to prime on the poly-adenine tails of RNA molecules. Alternatively, the terminal tags that are unique to each partition comprises of a sequencing adapter, a universal PCR sequence, a partition-specific barcode, a molecule-specific barcode, and/or a gene-specific sequence. FIG. 3 Terminal Tag Structure 2 is an exemplary adapter that can be useful in the present disclosure. The RNA molecules inside each partition can be tagged during reverse transcription using gene-specific sequence as the priming site to prime specific locations of the RNA molecules.

A reverse transcriptase can be used when RNA molecules are tagged with a partition-specific barcode and a molecule-specific barcode during reverse transcription inside a partition. The reverse transcriptase used for reverse transcription can add 2-5 cytosines at the end of the cDNA molecule. When the RNA molecules are barcoded by a reverse transcriptase with a terminal transferase and template-switching activity, template-switching oligonucleotides (TSO) that contain poly-guanines and a universal PCR priming sequence can be included. FIG. 3 Terminal Tag Structure 3 is an exemplary adapter that can be useful in the present disclosure. Template-switching and copying of the template-switching oligonucleotides can take place inside a partition after the reverse transcriptase reaches the 3′ end of the RNA molecule. The template-switching and copying of the template-switching oligonucleotides can take place after the partitions have been broken and cDNAs from all the partitions have been pooled.

When the RNA molecules are barcoded by a reverse transcriptase using terminal tags that contain both the partition-specific and molecule-specific barcode, an additional universal sequence can be appended on the opposing end of the terminal tag via primer elongation on the complementary DNA (cDNA) using a DNA polymerase. The primer for appending the universal sequence can also contain a gene-specific sequence that is downstream of the terminal tag. The addition of a second universal sequence can take place after the partitions have been broken and cDNAs from all the partitions have been pooled. When the RNA molecules are barcoded via a reverse transcriptase using terminal tags that contain both the partition-specific and molecule-specific barcode, an additional universal sequence can be appended on the opposing end of the terminal tag via adapter ligation using DNA ligase. The adapter containing a second universal sequence can be double-stranded and 5′ phosphorylated on one of the two strands. The ligation of the second universal sequence can take place after the partitions have been broken and cDNAs from all the partitions have been pooled.

The target for SLR sequencing can be an RNA molecule, and the terminal tags that are unique to each partition can comprise a sequencing adapter, a universal PCR sequence, a partition-specific barcode, a molecule-specific barcode, and/or a poly-guanine sequence. The RNA molecules inside each partition can be reverse-transcribed by a reverse transcriptase with a terminal transferase and template-switching activity using an oligo containing a universal PCR sequence and a poly-thymine sequence as the priming site to prime on the poly-adenine tails of RNA molecules. The partition-specific barcode and the molecule-specific barcode can be copied onto the cDNAs via template-switching activity of the reverse transcriptase. FIG. 3 Terminal Tag Structure 4 is an exemplary adapter that can be useful in the present disclosure. The oligonucleotides used for reverse transcription can contain a universal PCR sequence and a poly-thymine sequence, e.g. FIG. 3 Terminal Tag Structure 5. Alternatively, the oligonucleotides used for reverse transcription can contain a universal PCR sequence and a gene-specific sequence that primes at specific locations of the RNA molecules, e.g. FIG. 3 Terminal Tag Structure 6.

The terminal tags that are unique to each partition can also comprise a universal PCR sequence, a partition-specific barcode, and/or a poly-thymine sequence as the priming site to prime on the poly-adenine tails of the RNA molecules. A reverse transcriptase with a terminal transferase and template-switching activities can be used and can copy the sequence of a template-switching oligo containing poly-guanines, a molecule-specific barcode, a sequencing adapter, and a universal PCR sequence inside the partition. FIG. 3 Terminal Tag Structure 7 and Terminal Tag Structure 8 are exemplary adapters that can be useful in the present disclosure. The template-switching and copying of the template-switching oligonucleotides can take place after the partition have been broken and cDNAs from all the partitions have been pooled.

Alternatively, the terminal tags that are unique to each partition can comprise a universal PCR sequence, a partition-specific barcode, and/or gene-specific sequence as the priming site to prime on specific locations of the RNA molecules. A reverse transcriptase with a terminal transferase and template-switching activities can be used and can copy the sequence of a template-switching oligo containing poly-guanines, a molecule-specific barcode, a sequencing adapter, and/or a universal PCR sequence inside the partition. FIG. 3 Terminal Tag Structure 9 and Terminal Tag Structure 8 are exemplary adapters that can be useful in the present disclosure. The template-switching and copying of the template-switching oligonucleotides can take place after the partitions have been broken and cDNA from all the partitions have been pooled.

In other cases, the terminal tags that are unique to each partition can comprise a universal PCR sequence, a partition-specific barcode, and/or a poly-guanine sequence. The RNA molecules inside each partition can be reverse-transcribed by a reverse transcriptase with template-switching activity using an oligo containing a sequencing adapter, a universal PCR sequence, a molecule-specific barcode, and/or a poly-thymine sequence as the priming site to prime on the poly-adenine tails of RNA molecules. FIG. 3 Terminal Tag Structure 10 is an exemplary adapter that can be useful in the present disclosure. Partition-specific barcodes can be copied onto the cDNAs via the template-switching activity of the reverse transcriptase. The oligonucleotides used for reverse transcription can contain a sequencing adapter, a universal PCR sequence, and a poly-thymine sequence, e.g. FIG. 3 Terminal Tag Structure 11. Alternatively, the oligonucleotides used for reverse transcription can contain a sequencing adapter, a universal PCR sequence, a molecule-specific barcode, and/or a gene-specific sequence that primes at specific locations of the RNA molecules, e.g. FIG. 3 Terminal Tag Structure 12.

The poly-guanines used in template-switching oligonucleotides can be ribonucleotides, and the poly-guanosines used in template-switching oligonucleotides can be deoxynucleotides.

When the RNA molecules are barcoded by a reverse transcriptase using terminal tags that contain partition-specific barcode, the molecule-specific barcode can be appended on the opposing end of the terminal tag via primer elongation on the complementary DNA (cDNA) using a DNA polymerase. The primer for appending the molecule-specific barcode can also contain a gene-specific sequence that is downstream of the terminal tag and a universal sequence. The addition of the molecule-specific barcode can take place after the partitions have been broken and cDNAs from all the partitions are pooled. When the RNA molecules are barcoded via a reverse transcriptase using terminal tags that contain the partition-specific barcode, the molecule-specific barcode can be appended on the opposing end of the terminal tag via adapter ligation using DNA ligase. The adapter containing the molecule-specific barcode can also contain a universal sequence, can be double-stranded, and 5′ phosphorylated on one of the two strands. Ligation of the molecule-specific barcode can take place after the partitions have been broken and cDNAs from all the partitions are pooled.

DNA ligase used for adapter ligation of the universal sequence and/or molecule-specific barcode can include but is not limited to DNA ligase I, DNA ligase III, DNA ligase IV, and T4 DNA ligase.

Tagging of the RNA molecules inside each partition can be performed via single-stranded adapter ligation using T4 RNA ligase I. The terminal tags that are unique to each partition can comprise a sequencing adapter, a universal PCR sequence, a partition-specific barcode, and a molecule-specific barcode. The terminal tags can be 5′ phosphorylated and can contain a 3′ modification such as a linker spacer, an inverted base, or a dideoxynucleotide to prevent ligation of the terminal tags with each other. FIG. 3 Terminal Tag Structure 13 is an exemplary adapter that can be useful in the present disclosure.

The tagging of the RNA molecules inside each partition can be performed via single-stranded adapter ligation using T4 RNA ligase II truncated (T4 Rn12 truncated). The terminal tags that are unique to each partition can comprise a sequencing adapter, a universal PCR sequence, a partition-specific barcode, and/or a molecule-specific barcode. The terminal tags can be 5′ adenylated and can contain a 3′ modification such that two terminal tags cannot ligate with each other. FIG. 3 Terminal Tag Structure 14 is an exemplary adapter that can be useful in the present disclosure. The single-stranded adapter ligation to RNA molecules can be performed using 5′ App DNA/RNA ligase.

The targets for SLR sequencing can be DNA molecules, and the terminal tags that are unique to each partition can comprise a sequencing adapter, a universal PCR sequence, a partition-specific barcode, a molecule-specific barcode, and/or a gene-specific sequence. The DNA molecules inside each partition can be tagged via polymerase annealing-and-extension using the gene-specific sequence as the priming site to prime at specific locations of the DNA molecules.

The targets for SLR sequencing can be DNA molecules, and the terminal tags that are unique to each partition can comprise a sequencing adapter, a universal PCR sequence, a partition-specific barcode, a molecule-specific barcode, and/or a random sequence. The DNA molecules inside each partition can be tagged via polymerase annealing-and-extension using the random sequence as the priming site to prime at various and non-bias locations on the DNA molecules.

The targets for SLR sequencing can be DNA molecules, and the terminal tags that are unique to each partition can comprise a universal PCR sequence, a partition-specific barcode, and/or a gene-specific sequence. The DNA molecules inside each partition can be tagged via polymerase annealing-and-extension using the gene-specific sequence as the priming site to prime at specific locations of the DNA molecules. A second terminal tag comprising of a gene-specific sequence, a molecule-specific barcode, a sequencing adapter, and/or a universal PCR sequence can be used to barcode DNA molecules already tagged with partition-specific barcode inside the partition. The second tagging event with the molecule-specific barcode can take place after the partitions have been broken and the DNA from the partitions have been pooled. The gene-specific sequences on the terminal tags can bracket the region of interest in the DNA molecules for downstream amplification and phasing.

The targets for SLR sequencing can be DNA molecules, and the terminal tags that are unique to each partition can comprise a universal PCR sequence, a partition-specific barcode, and/or a random sequence. The DNA molecules inside each partition can be tagged via polymerase annealing-and-extension using the random sequence as the priming site to prime at various and non-bias locations on the DNA molecules. A second terminal tag comprising a random sequence, a molecule-specific barcode, a sequencing adapter, and/or a universal PCR sequence can be used to barcode DNA molecules already tagged with partition-specific barcode inside the partition using a DNA polymerase. A second tagging event with the molecule-specific barcode can occur after the partitions have been broken and the DNA from all the partitions are pooled.

The targets for SLR sequencing can be DNA molecules, and after cell lysis inside the partition, the DNA molecules inside each partition can be subject to enzymatic fragmentation into lengths that are longer than typical short-read sequencing read-lengths. After enzymatic fragmentation, terminal tags comprising a sequencing adapter, a universal PCR sequence, a partition-specific barcode, and/or a molecule-specific barcode can be ligated onto one of the terminal ends of the DNA long fragments using DNA ligase I. The barcode adapter can be double-stranded and 5′ phosphorylated on one of the two strands. FIG. 3 Terminal Tag Structure 15 is an exemplary adapter that can be useful in the present disclosure. The DNA molecules can be amplified prior to enzymatic fragmentation. The fragmented ends can be blunted prior to barcode adapter ligation.

Targets for SLR sequencing can be for example, DNA molecules. After cell lysis inside a partition, the DNA molecules inside each partition can be subject to enzymatic fragmentation into lengths that are longer than typical short-read sequencing read-lengths. After enzymatic fragmentation, terminal tags comprising a universal PCR sequence and a partition-specific barcode can be ligated onto one of the terminal ends of the DNA long fragments using DNA ligase I. A barcode adapter can be double-stranded and 5′ phosphorylated with a non-ligated 3′ end on one of the two strands. FIG. 3 Terminal Tag Structure 16 is an exemplary adapter that can be useful in the present disclosure. The DNA molecules can be amplified prior to enzymatic fragmentation. A second terminal tag comprising a sequencing adapter, a universal PCR sequence, and/or a molecule-specific barcode can then be ligated on the opposing end using DNA ligase, e.g. FIG. 3 Terminal Tag Structure 17. The second tagging event with the molecule-specific barcode can occur after the partitions have been broken and the DNA from all the partitions have been pooled.

The length of DNA molecules after fragmentation can be approximately 500-100000 base pairs. The length of the DNA molecules after fragmentation can be approximately 1000-50000 base pairs. The length of the DNA molecules after fragmentation can be approximately 2000-20000 base pairs. The length of DNA molecules after fragmentation can be about 500 base pairs to about 100,000 base pairs. The length of DNA molecules after fragmentation can be at least about 500 base pairs. The length of DNA molecules after fragmentation can be at most about 100,000 base pairs. For example, the length of DNA molecules after fragmentation can be about 500 base pairs to about 1,000 base pairs, about 500 base pairs to about 2,000 base pairs, about 500 base pairs to about 5,000 base pairs, about 500 base pairs to about 7,000 base pairs, about 500 base pairs to about 10,000 base pairs, about 500 base pairs to about 20,000 base pairs, about 500 base pairs to about 30,000 base pairs, about 500 base pairs to about 40,000 base pairs, about 500 base pairs to about 50,000 base pairs, about 500 base pairs to about 75,000 base pairs, about 500 base pairs to about 100,000 base pairs, about 1,000 base pairs to about 2,000 base pairs, about 1,000 base pairs to about 5,000 base pairs, about 1,000 base pairs to about 7,000 base pairs, about 1,000 base pairs to about 10,000 base pairs, about 1,000 base pairs to about 20,000 base pairs, about 1,000 base pairs to about 30,000 base pairs, about 1,000 base pairs to about 40,000 base pairs, about 1,000 base pairs to about 50,000 base pairs, about 1,000 base pairs to about 75,000 base pairs, about 1,000 base pairs to about 100,000 base pairs, about 2,000 base pairs to about 5,000 base pairs, about 2,000 base pairs to about 7,000 base pairs, about 2,000 base pairs to about 10,000 base pairs, about 2,000 base pairs to about 20,000 base pairs, about 2,000 base pairs to about 30,000 base pairs, about 2,000 base pairs to about 40,000 base pairs, about 2,000 base pairs to about 50,000 base pairs, about 2,000 base pairs to about 75,000 base pairs, about 2,000 base pairs to about 100,000 base pairs, about 5,000 base pairs to about 7,000 base pairs, about 5,000 base pairs to about 10,000 base pairs, about 5,000 base pairs to about 20,000 base pairs, about 5,000 base pairs to about 30,000 base pairs, about 5,000 base pairs to about 40,000 base pairs, about 5,000 base pairs to about 50,000 base pairs, about 5,000 base pairs to about 75,000 base pairs, about 5,000 base pairs to about 100,000 base pairs, about 7,000 base pairs to about 10,000 base pairs, about 7,000 base pairs to about 20,000 base pairs, about 7,000 base pairs to about 30,000 base pairs, about 7,000 base pairs to about 40,000 base pairs, about 7,000 base pairs to about 50,000 base pairs, about 7,000 base pairs to about 75,000 base pairs, about 7,000 base pairs to about 100,000 base pairs, about 10,000 base pairs to about 20,000 base pairs, about 10,000 base pairs to about 30,000 base pairs, about 10,000 base pairs to about 40,000 base pairs, about 10,000 base pairs to about 50,000 base pairs, about 10,000 base pairs to about 75,000 base pairs, about 10,000 base pairs to about 100,000 base pairs, about 20,000 base pairs to about 30,000 base pairs, about 20,000 base pairs to about 40,000 base pairs, about 20,000 base pairs to about 50,000 base pairs, about 20,000 base pairs to about 75,000 base pairs, about 20,000 base pairs to about 100,000 base pairs, about 30,000 base pairs to about 40,000 base pairs, about 30,000 base pairs to about 50,000 base pairs, about 30,000 base pairs to about 75,000 base pairs, about 30,000 base pairs to about 100,000 base pairs, about 40,000 base pairs to about 50,000 base pairs, about 40,000 base pairs to about 75,000 base pairs, about 40,000 base pairs to about 100,000 base pairs, about 50,000 base pairs to about 75,000 base pairs, about 50,000 base pairs to about 100,000 base pairs, or about 75,000 base pairs to about 100,000 base pairs. The length of DNA molecules after fragmentation can be about 500 base pairs, about 1,000 base pairs, about 2,000 base pairs, about 5,000 base pairs, about 7,000 base pairs, about 10,000 base pairs, about 20,000 base pairs, about 30,000 base pairs, about 40,000 base pairs, about 50,000 base pairs, about 75,000 base pairs, or about 100,000 base pairs.

DNA molecules can be amplified using a DNA polymerase and random primers of 6-20 bases long prior to random fragmentation and barcode ligation inside the partition. The DNA polymerase can amplify DNA molecules isothermally by annealing randomers to the DNA molecules, can amplify the template and displace the strand complementary to the template during DNA synthesis, and/or can generate partial single-stranded DNA regions that can then be used for additional primer annealing and extension.

The length of the random primers can be about 6 bases to about 20 bases. The length of the random primers can be at least about 6 bases. The length of the random primers can be at most about 20 bases. For example, the length of the random primers can be about 6 bases to about 7 bases, about 6 bases to about 8 bases, about 6 bases to about 9 bases, about 6 bases to about 10 bases, about 6 bases to about 11 bases, about 6 bases to about 12 bases, about 6 bases to about 15 bases, about 6 bases to about 17 bases, about 6 bases to about 18 bases, about 6 bases to about 19 bases, about 6 bases to about 20 bases, about 7 bases to about 8 bases, about 7 bases to about 9 bases, about 7 bases to about 10 bases, about 7 bases to about 11 bases, about 7 bases to about 12 bases, about 7 bases to about 15 bases, about 7 bases to about 17 bases, about 7 bases to about 18 bases, about 7 bases to about 19 bases, about 7 bases to about 20 bases, about 8 bases to about 9 bases, about 8 bases to about 10 bases, about 8 bases to about 11 bases, about 8 bases to about 12 bases, about 8 bases to about 15 bases, about 8 bases to about 17 bases, about 8 bases to about 18 bases, about 8 bases to about 19 bases, about 8 bases to about 20 bases, about 9 bases to about 10 bases, about 9 bases to about 11 bases, about 9 bases to about 12 bases, about 9 bases to about 15 bases, about 9 bases to about 17 bases, about 9 bases to about 18 bases, about 9 bases to about 19 bases, about 9 bases to about 20 bases, about 10 bases to about 11 bases, about 10 bases to about 12 bases, about 10 bases to about 15 bases, about 10 bases to about 17 bases, about 10 bases to about 18 bases, about 10 bases to about 19 bases, about 10 bases to about 20 bases, about 11 bases to about 12 bases, about 11 bases to about 15 bases, about 11 bases to about 17 bases, about 11 bases to about 18 bases, about 11 bases to about 19 bases, about 11 bases to about 20 bases, about 12 bases to about 15 bases, about 12 bases to about 17 bases, about 12 bases to about 18 bases, about 12 bases to about 19 bases, about 12 bases to about 20 bases, about 15 bases to about 17 bases, about 15 bases to about 18 bases, about 15 bases to about 19 bases, about 15 bases to about 20 bases, about 17 bases to about 18 bases, about 17 bases to about 19 bases, about 17 bases to about 20 bases, about 18 bases to about 19 bases, about 18 bases to about 20 bases, or about 19 bases to about 20 bases. The length of the random primers can be about 6 bases, about 7 bases, about 8 bases, about 9 bases, about 10 bases, about 11 bases, about 12 bases, about 15 bases, about 17 bases, about 18 bases, about 19 bases, or about 20 bases.

A partition-specific barcode in a terminal tag can be comprised entirely of a random sequence and the many copies of the barcode within each partition can be identical. Alternatively, a partition-specific barcode in a terminal tag can be comprised of a combination of a random sequence and a known sequence. The known sequence can be used to identify the sample from which the cell partitions can be made. A partition-specific barcode in a terminal tag can be comprised of an entirely known sequence, including a partition-specific sequence, or both a partition-specific sequence and a sample-specific sequence.

Nucleic acid molecules can be tagged with a partition-specific barcode, which can contain a sample-specific barcode. A second tagging including, for example, a molecule-specific barcode can also occur. The second tagging can occur as a bulk single reaction, i.e. each sample from which the cell partitions are made can be tagged separately, or as a bulk multiplexed reaction, i.e. multiple samples from which different cell partitions are made, each pool with a different sample-specific sequence, can be tagged together.

A molecule-specific terminal adapter can be present at both ends of a long nucleic acid molecule. A molecule-specific terminal adapter can be present at one end of a long nucleic acid molecule. The location of a molecule-specific terminal adapter can be upstream of a long nucleic acid molecule. Alternatively, the location of a molecule-specific terminal adapter can be downstream of a long nucleic acid molecule.

As used herein, “molecule-specific barcode” and “molecular barcode” can be used interchangeably. A molecule-specific barcode or a molecular barcode in a terminal tag can comprise an entirely random sequence. A molecular barcode in a terminal tag can comprise a semi-random sequence, for example, a combination of a random molecule-specific sequence and a known sequence, wherein the known sequence is used to identify the sample from which multiple parental nucleic acid sequences originate. Alternatively, a molecular barcode in a terminal tag can comprise an entirely known sequence, including a molecule-specific sequence, or both a molecule-specific sequence and a sample-specific sequence.

An elongation sequence can comprise an entirely random sequence. An elongation sequence can comprise a combination of a random molecule-specific sequence and a known sequence, wherein the known sequence is used to identify the sample from which multiple parental nucleic sequences originate. An elongation sequence can comprise an entirely known sequence, including a molecule-specific sequence, or both a molecule-specific sequence and a sample-specific sequence. An elongation sequence can comprise a substantial or complete complementarity to a portion of a target nucleic acid sequence. An elongation sequence can comprise a partial complementarity to a portion of a target nucleic acid sequence. An elongation sequence can comprise, for example, at least about: 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% complementarity to a portion of a target nucleic acid sequence that it anneals to.

A barcode sequence, used to identify individual nucleic acid molecules as to their partition origin or used to identify short read sequences of their long molecule origin, can have a length of about 10-50 bp, about 15-30 bp, or about 20-25 bp. A barcode sequence can have a length of about 10 bp, about 20 bp, about 30 bp, about 40 bp, or about 50 bp. A barcode sequence can have a length of about 15 bp, 20 bp, 25 bp, or 30 bp. A barcode sequence can have a length of about 20 bp or about 25 bp. The length of a barcode sequence can be about 10 base pairs (bp) to about 50 base pairs (bp). A barcode sequence can have a length of about 5 bp to about 50 bp. A barcode sequence can have a length of at least about 5 bp. A barcode sequence can have a length of at most about 50 bp. The length of a barcode sequence can be at least about 10 base pairs. The length of a barcode sequence can be at most about 50 base pairs. For example, a barcode sequence can have a length of about 5 bp to about 10 bp, about 5 bp to about 15 bp, about 5 bp to about 20 bp, about 5 bp to about 25 bp, about 5 bp to about 30 bp, about 5 bp to about 35 bp, about 5 bp to about 40 bp, about 5 bp to about 45 bp, about 5 bp to about 50 bp. The length of a barcode sequence can be about 10 base pairs to about 15 base pairs, about 10 base pairs to about 17 base pairs, about 10 base pairs to about 19 base pairs, about 10 base pairs to about 22 base pairs, about 10 base pairs to about 25 base pairs, about 10 base pairs to about 27 base pairs, about 10 base pairs to about 30 base pairs, about 10 base pairs to about 35 base pairs, about 10 base pairs to about 40 base pairs, about 10 base pairs to about 45 base pairs, about 10 base pairs to about 50 base pairs, about 15 base pairs to about 17 base pairs, about 15 base pairs to about 19 base pairs, about 15 base pairs to about 22 base pairs, about 15 base pairs to about 25 base pairs, about 15 base pairs to about 27 base pairs, about 15 base pairs to about 30 base pairs, about 15 base pairs to about 35 base pairs, about 15 base pairs to about 40 base pairs, about 15 base pairs to about 45 base pairs, about 15 base pairs to about 50 base pairs, about 17 base pairs to about 19 base pairs, about 17 base pairs to about 22 base pairs, about 17 base pairs to about 25 base pairs, about 17 base pairs to about 27 base pairs, about 17 base pairs to about 30 base pairs, about 17 base pairs to about 35 base pairs, about 17 base pairs to about 40 base pairs, about 17 base pairs to about 45 base pairs, about 17 base pairs to about 50 base pairs, about 19 base pairs to about 22 base pairs, about 19 base pairs to about 25 base pairs, about 19 base pairs to about 27 base pairs, about 19 base pairs to about 30 base pairs, about 19 base pairs to about 35 base pairs, about 19 base pairs to about 40 base pairs, about 19 base pairs to about 45 base pairs, about 19 base pairs to about 50 base pairs, about 22 base pairs to about 25 base pairs, about 22 base pairs to about 27 base pairs, about 22 base pairs to about 30 base pairs, about 22 base pairs to about 35 base pairs, about 22 base pairs to about 40 base pairs, about 22 base pairs to about 45 base pairs, about 22 base pairs to about 50 base pairs, about 25 base pairs to about 27 base pairs, about 25 base pairs to about 30 base pairs, about 25 base pairs to about 35 base pairs, about 25 base pairs to about 40 base pairs, about 25 base pairs to about 45 base pairs, about 25 base pairs to about 50 base pairs, about 27 base pairs to about 30 base pairs, about 27 base pairs to about 35 base pairs, about 27 base pairs to about 40 base pairs, about 27 base pairs to about 45 base pairs, about 27 base pairs to about 50 base pairs, about 30 base pairs to about 35 base pairs, about 30 base pairs to about 40 base pairs, about 30 base pairs to about 45 base pairs, about 30 base pairs to about 50 base pairs, about 35 base pairs to about 40 base pairs, about 35 base pairs to about 45 base pairs, about 35 base pairs to about 50 base pairs, about 40 base pairs to about 45 base pairs, about 40 base pairs to about 50 base pairs, or about 45 base pairs to about 50 base pairs. The length of a barcode sequence can be about 5 base pairs, about 10 base pairs, about 15 base pairs, about 17 base pairs, about 19 base pairs, about 22 base pairs, about 25 base pairs, about 27 base pairs, about 30 base pairs, about 35 base pairs, about 40 base pairs, about 45 base pairs, or about 50 base pairs.

The universal sequences on the 5′ terminal tag and the 3′ terminal tag can be the same sequence. Alternatively, the universal sequence on the 5′ terminal tag can be different from the universal sequence on the 3′ terminal tag. DNA and RNA molecules can be tagged with both partition-specific barcodes and molecule-specific barcodes. Several copies of the uniquely tagged nucleic acid molecules can be obtained via, for example, PCR amplification using the universal sequence regions in the terminal tags. PCR amplification can be used to generate multiple copies of the uniquely tagged nucleic acid molecules, for example, by using primers containing uracil and an uracil-tolerant polymerase. The uracil-tolerant polymerase can also contain proof-reading activities. Uracil-tolerant polymerase can use uracil-containing primers to initiate elongation and/or to incorporate uracil during DNA extension.

The primers used to amplify tagged nucleic acid molecules can contain uracil. Thus, the universal priming region can be removed after PCR amplification using a combination of an uracil-DNA glycosylase to remove the uracil base, and an endonuclease such as Endonuclease VIII to remove the apurinic/apyrimidinic site. An exonuclease such as T4 DNA polymerase or DNA polymerase I large fragment can be used to remove the sequence complementary to the universal priming region.

The PCR amplification of the pool of uniquely tagged nucleic acid molecules can be conducted using oligonucleotides comprising both the universal sequence and a gene-specific sequence. The gene-specific sequence can be a sequence within the tagged DNA molecules. The gene-specific sequence can include sequences that can be used to tag the nucleic acid molecules at the terminal ends. One or more primers containing a different gene-specific sequence can be used for PCR amplification of the uniquely tagged nucleic acid. The gene-specific sequence can be used to perform intramolecular priming and elongation reaction using a DNA polymerase. Specifically, the gene-specific sequence can be the reverse complement of an internal sequence and can serve as a primer for intramolecular-elongation. The gene-specific sequences can span the length of the internal nucleic acid molecule so as to provide sequence coverage of the entire long molecule in the short-read sequencing library.

The PCR amplification of the pool of uniquely tagged nucleic acid molecules can be conducted using oligonucleotides comprising both the universal sequence and a short random sequence. The short random sequence can comprise 6-20 random nucleotides and can be used to perform intramolecular priming and elongation reaction at random locations within the tagged nucleic acid molecule using a DNA polymerase. The random sequence primer can span the length of the internal nucleic acid molecule at various locations, thus providing sequence coverage of the entire long molecule in the short-read sequencing library.

Where the PCR amplification of the pool of uniquely tagged nucleic acid molecules is conducted using oligonucleotides comprising both the universal sequence and a gene-specific or random sequence, the gene-specific or random sequence can be appended to the terminal tag that contains the molecule-specific barcode. In addition, a second primer comprising a different universal sequence can be used for PCR amplification of the pool of uniquely tagged nucleic acid molecules and/or can dictate the terminal end that the gene-specific or random sequence can be appended to. The PCR amplification of the pool of uniquely tagged nucleic acid molecules can occur in a single reaction, i.e. each sample from which the cell partitions are made can be amplified individually, or as a multiplexed reaction, i.e. multiple samples from which different cell partitions are made, each pool with a different sample-specific sequence, can be amplified.

The PCR amplified pool of uniquely tagged DNA molecules can be fragmented at random locations within the nucleic acid molecules and result in fragments that contain either the 5′ terminal tag, the 3′ terminal tag, or devoid of tags. The average rate of fragmentation can be chosen such that the pool of library includes both fragmented and unfragmented nucleic acid molecules. An exonuclease or a DNA polymerase with a strong single-stranded exonuclease activity can be used to generate blunt ends in the newly fragmented nucleic acid molecules. The pool of tagged and fragmented DNA molecules can then be circularized by intramolecular ligation under dilute conditions using DNA ligase. The DNA molecules can be fragmented at random locations prior to circularization. The partition-specific and/or molecule-specific barcodes at the terminal tags can be effectively distributed, or made proximate, to various locations within the DNA molecules. The various locations which the barcodes are distributed to can provide coverage that span the entire length of the long molecule in the short-read sequencing library.

The tagged and amplified DNA molecules can be fragmented into fragments, each having a different length. The fragmentation can be performed by enzymatic fragmentation methods, sonication-based fragmentation, acoustic shearing, nebulization, needle shearing and French pressure cells, or any combination thereof. The fragmented DNA can be blunted. Blunt ends can be generated using a single strand-specific DNA exonuclease, such as exonuclease I, exonuclease VII, or a combination thereof, thus, degrading the overhanging single stranded ends. In addition, blunt ends can be generated using a single strand-specific DNA endonuclease, such as mung bean endonuclease or S1 endonuclease. Blunt ends can be generated using a polymerase that comprises single stranded exonuclease activity, such as T4 DNA polymerase, any other polymerase comprising single stranded exonuclease activity, or a combination thereof. Blunted DNA can be 5′ phosphorylated using T4 polynucleotide kinase. The 5′ phosphorylation can be important for subsequent intramolecular ligation of the tagged DNA fragments. Alternatively, blunted DNA can be 5′ phosphorylated by incorporating dUTP in the terminal adpters. The 5′ phosphorylation site can be generated using a combination of uracil-DNA glycosylase and an endonuclease to hydrolyze the apurinic/apyrimidinic sites. The uracil-DNA glycosylase can be E coli uracil-DNA glycosylase.

The PCR amplified pool of uniquely tagged double stranded DNA (dsDNA) molecules can be turned into single-stranded DNA (ssDNA) molecules via heat denaturation under dilute conditions. The gene-specific or random sequence at the 3′ end of the terminal tag can be used to intramolecularly prime and elongate at either specific locations or random locations within the long ssDNA molecule under dilute conditions using a DNA polymerase. Different gene-specific or random sequence can be used for intramolecular elongation. The partition-specific and/or molecule-specific barcodes at the terminal tags can be effectively distributed, or made proximate, to various locations within the DNA molecules. The gene-specific or random sequences can provide coverage that span the entire length of the long molecule or specific regions of interest within the long molecule in the short-read sequencing library. The locations of the gene-specific sites can be separated by a distance that is approximately the read-length of the short-read sequencer.

Prior to the intramolecular-elongation, the pool of uniquely tagged nucleic acids can be truncated to smaller fragments, such as ssDNA or dsDNA. The terminal tag with 3′ gene-specific or random sequence can be intramolecularly-elongated using a DNA polymerase to produce a pool of uniquely tagged double-stranded DNA (dsDNA) of varying lengths. The length of DNA extension during the intramolecular elongation can be limited to approximately the read length of NGS. The intramolecular elongation generating DNA of various lengths can occur in parallel reactions, e.g. multiple PCR reactions with the same reagent composition or with a different primer composition in each reaction, or can occur in a multiplexed reaction, e.g. PCR reactions with different primer compositions in the same reaction. Once the terminal tags containing partition-specific barcodes and/or molecule-specific barcodes are distributed to various locations within the long nucleic acid molecules, either via intramolecular ligation or intramolecular elongation, the pool of nucleic acid molecules can be prepared for NGS using standard NGS library preparation and/or PCR amplification.

Standard NGS library preparation used for converting nucleic acid molecules with partition-specific barcodes and/or molecule-specific barcodes distributed to various locations can include fragmentation of the nucleic acid molecules to a size that is approximately the read-length of the short-read sequencer, end-repairing the fragmentation sites to blunt ends, a-tailing the fragment ends in preparation for TA ligation, and ligating with ligation adapters that can include a second sequencing adapter. Consequently, the pool of nucleic acid molecules containing two sequencing adapters can be PCR amplified to append additional universal sequencing sequences, e.g. Illumina's P5 and P7 sequences, as well as a second sample index to differentiate between different pools of nucleic acid molecules on the short-read sequencer.

The a-tailing step during NGS library preparation can be eliminated if the ligation adapters are bunt-ended and designed such that they do not self-ligate by for example, including un-ligatable 3′ ends on the ligation adapter. The second sequencing adapter ligated during NGS library preparation can contain a second sample index to differentiate between different pools of nucleic acid molecules on the short-read sequencer. The final library amplification can append to the universal sequencing sequences, e.g. Illumina's P5 and P7 sequences.

The NGS library preparation for converting nucleic acid molecules with partition-specific barcodes and/or molecule-specific barcodes distributed to various locations can include PCR amplification with one or more primers, each containing a second sequencing adapter and a different gene-specific site. Collectively, the gene-specific sites can provide coverage that spans the length of the long nucleic acid molecules or specific regions of interest. The locations of the gene-specific sites can be separated by a distance that is approximately the read-length of the short-read sequencer. The pool of nucleic acid molecules containing two sequencing adapters can then be PCR amplified to append additional universal sequencing sequences, e.g. Illumina's P5 and P7 sequences, as well as a second sample index to differentiate between different pools of nucleic acid molecules on the short-read sequencer. The second sequencing adapter appended via PCR amplification during NGS library preparation can contain a second sample index to differentiate between different pools of nucleic acid molecules on the short-read sequencer. The final library amplification can append to the universal sequencing sequences, e.g. Illumina's P5 and P7 sequences. When the terminal tag includes a 3′ gene-specific sequence for intramolecular elongation, the gene-specific sites used during NGS library preparation can be downstream of the gene-specific sites used for intramolecular-elongation. The distance between the gene-specific sites used for intramolecular-elongation and the gene-specific sites used for NGS library preparation can be, approximately, the read-length of the short-read sequencer.

The partition-specific and molecule-specific terminal tag can be present at one end of the long nucleic acid molecules. Alternatively, the partition-specific terminal tag can be present on one end of the nucleic acid molecules while the molecule-specific terminal tag can be present on the other end of the nucleic acid molecules. In other cases, the partition-specific and molecule-specific terminal tag can be present at both ends of the long nucleic acid molecules. The location of the partition-specific and/or molecule-specific terminal tag(s) can be upstream or downstream of the long nucleic acid molecules.

The intramolecular-ligation can distribute barcodes without bias to loci. The loci can be evenly distributed throughout the long nucleic acid molecule such that that the loci of interests are adjacent to and share the same molecule-specific barcode if they originate from the same single long molecule. The loci can be separated by 200-10000 base pairs such that the loci of interests on the same single long molecule can share the same molecule-specific barcode. In addition, the barcoded NGS short reads constructed from the intramolecularly-ligated library can provide sequence coverage for the entire long nucleic acid molecule and generate contiguous synthetic long reads for phasing. The intramolecular-elongation can copy, without bias, loci that are evenly distributed throughout the long nucleic acid molecule such that that the loci of interests are adjacent to and share the same molecule-specific barcode if they originate from the same single long molecule.

The barcoded NGS short reads constructed from the intramolecularly-elongated library can provide sequence coverage for the entire long nucleic acid molecule and generate contiguous synthetic long reads for phasing. Alternatively, the barcoded NGS short reads constructed from the intramolecularly-elongated library can cover regions of interests that are separated by homologous regions and generate discontiguous synthetic long reads for phasing.

The intramolecular-elongation sequence in the terminal adpter tag can be at the 3′-end and/or can comprise a sequence selected from a target-specific self-elongation sequence or a random sequence. The self-elongation sequence at the 3′-end of the molecule-specific terminal adpter can be a target sequence complementary to an internal sequence of the uniquely barcoded and elongation-primed ssDNA molecules in the mixture. Blunt end ligation, TA ligation, or primer extension can be used to append the long nucleic acid molecules in the mixture with unique tags containing molecule-specific barcodes and self-elongation sequences. The mixture of nucleic acid molecules can be appended with unique tags by carrying out PCR with primers containing the unique tag. The mixture of nucleic acid molecules can be appended with unique tags by adding the unique tag to the terminals during DNA synthesis. Sequence independent tagging can be performed during DNA synthesis to obtain synthesized DNA sequences flanked with barcode tags. Barcoding of the synthetic DNA can be used in the quality control thereof.

In some aspects, the long nucleic acid molecules in the mixture may be appended with unique tags that contain both the molecule-specific barcode and the self-elongation sequence. In some aspects, the long nucleic acid molecules in the mixture may be appended with unique tags that contain the molecule-specific barcode but not the self-elongation sequence.

The initial tagging of a mixture of single nucleic acid molecules with unique tags can include, for example, carrying out a PCR with primers containing a molecule-specific tag. The PCR can be performed by using primers that contain molecule-specific tags. Alternatively, the PCR can be performed by using only one primer that contains a molecule-specific tag. The PCR can be performed with an oligonucleotide that comprises a complement of a first adpter. Alternatively, the PCR can be performed with an oligonucleotide that comprises a reverse complement of the first adpter and a sequence complementary to at least a portion of a template nucleic acid. The 3′ end of the nucleotide can comprise a sequence complementary to at least a portion of a template nucleic acid. Alternatively, the PCR can be performed with an oligonucleotide that comprises a complement of the first adpter and a sequence complementary to at least a portion of a template nucleic acid, wherein the sequence complementary to at least a portion of the template nuclei acid comprises a random sequence or a complete complementary to the portion of the template nuclei acid.

Tagged double-stranded DNA (dsDNA) can be subject to heat denaturation under dilute condition in preparation for single-strand DNA (ssDNA) intramolecular-elongation. Intramolecular annealing and elongation can be more efficient than intermolecular annealing (two complementary strands annealing back together). Tagged dsDNA can be selectively phosphorylated at one of its 5′ termini; ssDNA can be prepared for intramolecular elongation from the dsDNA through the use of an exonuclease such as Lambda exonuclease that selectively degrades the 5′ phosphorylated strands. The tagged dsDNA can be bound to a streptavidin-coated solid surface, such as streptavidin magnetic beads, through a 5′ biotin primer modification and ssDNA is prepared for intramolecular elongation from the non-bound opposite strand by washing off the unbound strand from the beads either by heat denaturation or alkaline denaturation.

PCR primer extension after intramolecular elongation, or enrichment PCR, can occur in parallel reactions. Enrichment PCR can occur in multiple PCR reactions, wherein each reaction has a different primer composition. Alternatively, enrichment PCR can occur in a multiplexed reaction, wherein PCR reactions occur with multiple primers in the same reaction. Enrichment PCR can include multiple primers (e.g., a multiplexed reaction), wherein each primer can have a different target sequence that can be complementary to the sequence downstream of an elongation locus and a universal sequencing adapter. Enrichment PCR can be performed as a multiplexed reaction using primers with different target sequences. The amplified elongation products can contain one or more products from all the target sequences downstream of each elongation locus. Collectively, the elongation products can represent from one or more combinations of elongation loci and target sequences downstream of each elongation locus. The distance between an elongation locus and a target sequence in the enrichment PCR can be approximately one read-length apart. Alternatively, the distance between an elongation locus and a target sequence in the enrichment PCR can be approximately 100 bp, 150 bp, 200 bp, 250 bp, 300 bp, 350 bp, 400 bp, 450 bp, or 500 bp apart.

The distance between an elongation locus and a target sequence can be about 100 base pairs to about 500 base pairs. The distance between an elongation locus and a target sequence can be at least about 100 base pairs. The distance between an elongation locus and a target sequence can be at most about 500 base pairs. The distance between an elongation locus and a target sequence can be about 100 base pairs to about 150 base pairs, about 100 base pairs to about 170 base pairs, about 100 base pairs to about 190 base pairs, about 100 base pairs to about 220 base pairs, about 100 base pairs to about 250 base pairs, about 100 base pairs to about 270 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 150 base pairs to about 170 base pairs, about 150 base pairs to about 190 base pairs, about 150 base pairs to about 220 base pairs, about 150 base pairs to about 250 base pairs, about 150 base pairs to about 270 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 170 base pairs to about 190 base pairs, about 170 base pairs to about 220 base pairs, about 170 base pairs to about 250 base pairs, about 170 base pairs to about 270 base pairs, about 170 base pairs to about 300 base pairs, about 170 base pairs to about 350 base pairs, about 170 base pairs to about 400 base pairs, about 170 base pairs to about 450 base pairs, about 170 base pairs to about 500 base pairs, about 190 base pairs to about 220 base pairs, about 190 base pairs to about 250 base pairs, about 190 base pairs to about 270 base pairs, about 190 base pairs to about 300 base pairs, about 190 base pairs to about 350 base pairs, about 190 base pairs to about 400 base pairs, about 190 base pairs to about 450 base pairs, about 190 base pairs to about 500 base pairs, about 220 base pairs to about 250 base pairs, about 220 base pairs to about 270 base pairs, about 220 base pairs to about 300 base pairs, about 220 base pairs to about 350 base pairs, about 220 base pairs to about 400 base pairs, about 220 base pairs to about 450 base pairs, about 220 base pairs to about 500 base pairs, about 250 base pairs to about 270 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 270 base pairs to about 300 base pairs, about 270 base pairs to about 350 base pairs, about 270 base pairs to about 400 base pairs, about 270 base pairs to about 450 base pairs, about 270 base pairs to about 500 base pairs, about 300 base pairs to about 350 base pairs, about 300 base pairs to about 400 base pairs, about 300 base pairs to about 450 base pairs, about 300 base pairs to about 500 base pairs, about 350 base pairs to about 400 base pairs, about 350 base pairs to about 450 base pairs, about 350 base pairs to about 500 base pairs, about 400 base pairs to about 450 base pairs, about 400 base pairs to about 500 base pairs, or about 450 base pairs to about 500 base pairs. The distance between an elongation locus and a target sequence can be about 100 base pairs, about 150 base pairs, about 170 base pairs, about 190 base pairs, about 220 base pairs, about 250 base pairs, about 270 base pairs, about 300 base pairs, about 350 base pairs, about 400 base pairs, about 450 base pairs, or about 500 base pairs.

When the enrichment PCR is performed as a multiplexed reaction, the loci used for intramolecular elongation can be different from the target sequences used in enrichment PCR. The distance between any elongation locus and any downstream target sequence can be at least about 10-50 bp apart. Alternatively, the distance between any elongation locus and any downstream target sequence can be at least about 50-100 bp apart. When the enrichment PCR is performed as a multiplexed reaction, the loci used for intramolecular elongation can be different from the target sequences used in the enrichment PCR. When the enrichment PCR is performed as a multiplexed reaction, the distance between any elongation locus and any downstream target sequence can be at least about 10 bp, 15 bp, 20 bp, 25 bp, 30 bp, 35 bp, 40 bp, 45 bp, or 50 bp apart. Alternatively, when the enrichment PCR is performed as a multiplexed reaction, the distance between any elongation locus and any downstream target sequence can be at least about 50 bp, 55 bp, 60 bp, 65 bp, 70 bp, 75 bp, 80 bp, 85 bp, 90 bp, 95 bp, or 100 bp apart.

The distance between an elongation locus and a downstream target sequence can be about 10 bp to about 100 bp. The distance between an elongation locus and a downstream target sequence can be about 10 base pairs to about 50 base pairs. The distance between an elongation locus and a downstream target sequence can be at least about 10 base pairs. The distance between an elongation locus and a downstream target sequence can be at most about 50 base pairs. The distance between an elongation locus and a downstream target sequence can be at most about 100 bp. The distance between an elongation locus and a downstream target sequence can be about 10 base pairs to about 15 base pairs, about 10 base pairs to about 17 base pairs, about 10 base pairs to about 19 base pairs, about 10 base pairs to about 22 base pairs, about 10 base pairs to about 25 base pairs, about 10 base pairs to about 27 base pairs, about 10 base pairs to about 30 base pairs, about 10 base pairs to about 35 base pairs, about 10 base pairs to about 40 base pairs, about 10 base pairs to about 45 base pairs, about 10 base pairs to about 50 base pairs, about 15 base pairs to about 17 base pairs, about 15 base pairs to about 19 base pairs, about 15 base pairs to about 22 base pairs, about 15 base pairs to about 25 base pairs, about 15 base pairs to about 27 base pairs, about 15 base pairs to about 30 base pairs, about 15 base pairs to about 35 base pairs, about 15 base pairs to about 40 base pairs, about 15 base pairs to about 45 base pairs, about 15 base pairs to about 50 base pairs, about 17 base pairs to about 19 base pairs, about 17 base pairs to about 22 base pairs, about 17 base pairs to about 25 base pairs, about 17 base pairs to about 27 base pairs, about 17 base pairs to about 30 base pairs, about 17 base pairs to about 35 base pairs, about 17 base pairs to about 40 base pairs, about 17 base pairs to about 45 base pairs, about 17 base pairs to about 50 base pairs, about 19 base pairs to about 22 base pairs, about 19 base pairs to about 25 base pairs, about 19 base pairs to about 27 base pairs, about 19 base pairs to about 30 base pairs, about 19 base pairs to about 35 base pairs, about 19 base pairs to about 40 base pairs, about 19 base pairs to about 45 base pairs, about 19 base pairs to about 50 base pairs, about 22 base pairs to about 25 base pairs, about 22 base pairs to about 27 base pairs, about 22 base pairs to about 30 base pairs, about 22 base pairs to about 35 base pairs, about 22 base pairs to about 40 base pairs, about 22 base pairs to about 45 base pairs, about 22 base pairs to about 50 base pairs, about 25 base pairs to about 27 base pairs, about 25 base pairs to about 30 base pairs, about 25 base pairs to about 35 base pairs, about 25 base pairs to about 40 base pairs, about 25 base pairs to about 45 base pairs, about 25 base pairs to about 50 base pairs, about 27 base pairs to about 30 base pairs, about 27 base pairs to about 35 base pairs, about 27 base pairs to about 40 base pairs, about 27 base pairs to about 45 base pairs, about 27 base pairs to about 50 base pairs, about 30 base pairs to about 35 base pairs, about 30 base pairs to about 40 base pairs, about 30 base pairs to about 45 base pairs, about 30 base pairs to about 50 base pairs, about 35 base pairs to about 40 base pairs, about 35 base pairs to about 45 base pairs, about 35 base pairs to about 50 base pairs, about 40 base pairs to about 45 base pairs, about 40 base pairs to about 50 base pairs, or about 45 base pairs to about 50 base pairs. The distance between an elongation locus and a downstream target sequence can be about 10 bp to about 60 bp, about 10 bp to about 70 bp, about 10 bp to about 80 bp, about 10 bp to about 90 bp, about 10 bp to about 100 bp, about 20 bp to about 60 bp, about 20 bp to about 70 bp, about 20 bp to about 80 bp, about 20 bp to about 90 bp, about 20 bp to about 100 bp, about 30 bp to about 60 bp, about 30 bp to about 70 bp, about 30 bp to about 80 bp, about 30 bp to about 90 bp, about 30 bp to about 100 bp, about 40 bp to about 60 bp, about 40 bp to about 70 bp, about 40 bp to about 80 bp, about 40 bp to about 90 bp, about 40 bp to about 100 bp, about 50 bp to about 60 bp, about 50 bp to about 70 bp, about 50 bp to about 80 bp, about 50 bp to about 90 bp, about 50 bp to about 100 bp, about 60 bp to about 70 bp, about 60 bp to about 80 bp, about 60 bp to about 90 bp, about 60 bp to about 100 bp, about 70 bp to about 80 bp, about 70 bp to about 90 bp, about 70 bp to about 100 bp, about 80 bp to about 90 bp, about 80 bp to about 100 bp, or about 90 bp to about 100 bp. The distance between an elongation locus and a downstream target sequence can be about 10 bp, about 20 bp, about 30 bp, about 40 bp, about 50 bp, about 60 bp, about 70 bp, about 80 bp, about 90 bp, or about 100 bp. The distance between an elongation locus and a downstream target sequence can be about 10 base pairs, about 15 base pairs, about 17 base pairs, about 19 base pairs, about 22 base pairs, about 25 base pairs, about 27 base pairs, about 30 base pairs, about 35 base pairs, about 40 base pairs, about 45 base pairs, about 50 base pairs, about 60 bp, about 70 bp, about 80 bp, about 90 bp, or about 100 bp.

The average length of the nucleic acid molecules that are tagged with partition-specific and/or molecule-specific barcodes can be in the range of about 500-5000 base pairs. Alternatively, the average length of the nucleic acid molecules to be tagged can be in the range of about 1000-10000 base pairs.

The length of the nucleic acid molecules to be tagged can be about 500 base pairs to about 15,000 base pairs. For example, the length of the nucleic acid molecules to be tagged can be at least about 500 base pairs or at most about 15,000 base pairs. Specifically, the length of the nucleic acid molecules to be tagged can be about 500 base pairs to about 1,000 base pairs, about 500 base pairs to about 2,000 base pairs, about 500 base pairs to about 3,000 base pairs, about 500 base pairs to about 4,000 base pairs, about 500 base pairs to about 5,000 base pairs, about 500 base pairs to about 6,000 base pairs, about 500 base pairs to about 7,000 base pairs, about 500 base pairs to about 8,000 base pairs, about 500 base pairs to about 9,000 base pairs, about 500 base pairs to about 10,000 base pairs, about 500 base pairs to about 15,000 base pairs, about 1,000 base pairs to about 2,000 base pairs, about 1,000 base pairs to about 3,000 base pairs, about 1,000 base pairs to about 4,000 base pairs, about 1,000 base pairs to about 5,000 base pairs, about 1,000 base pairs to about 6,000 base pairs, about 1,000 base pairs to about 7,000 base pairs, about 1,000 base pairs to about 8,000 base pairs, about 1,000 base pairs to about 9,000 base pairs, about 1,000 base pairs to about 10,000 base pairs, about 1,000 base pairs to about 15,000 base pairs, about 2,000 base pairs to about 3,000 base pairs, about 2,000 base pairs to about 4,000 base pairs, about 2,000 base pairs to about 5,000 base pairs, about 2,000 base pairs to about 6,000 base pairs, about 2,000 base pairs to about 7,000 base pairs, about 2,000 base pairs to about 8,000 base pairs, about 2,000 base pairs to about 9,000 base pairs, about 2,000 base pairs to about 10,000 base pairs, about 2,000 base pairs to about 15,000 base pairs, about 3,000 base pairs to about 4,000 base pairs, about 3,000 base pairs to about 5,000 base pairs, about 3,000 base pairs to about 6,000 base pairs, about 3,000 base pairs to about 7,000 base pairs, about 3,000 base pairs to about 8,000 base pairs, about 3,000 base pairs to about 9,000 base pairs, about 3,000 base pairs to about 10,000 base pairs, about 3,000 base pairs to about 15,000 base pairs, about 4,000 base pairs to about 5,000 base pairs, about 4,000 base pairs to about 6,000 base pairs, about 4,000 base pairs to about 7,000 base pairs, about 4,000 base pairs to about 8,000 base pairs, about 4,000 base pairs to about 9,000 base pairs, about 4,000 base pairs to about 10,000 base pairs, about 4,000 base pairs to about 15,000 base pairs, about 5,000 base pairs to about 6,000 base pairs, about 5,000 base pairs to about 7,000 base pairs, about 5,000 base pairs to about 8,000 base pairs, about 5,000 base pairs to about 9,000 base pairs, about 5,000 base pairs to about 10,000 base pairs, about 5,000 base pairs to about 15,000 base pairs, about 6,000 base pairs to about 7,000 base pairs, about 6,000 base pairs to about 8,000 base pairs, about 6,000 base pairs to about 9,000 base pairs, about 6,000 base pairs to about 10,000 base pairs, about 6,000 base pairs to about 15,000 base pairs, about 7,000 base pairs to about 8,000 base pairs, about 7,000 base pairs to about 9,000 base pairs, about 7,000 base pairs to about 10,000 base pairs, about 7,000 base pairs to about 15,000 base pairs, about 8,000 base pairs to about 9,000 base pairs, about 8,000 base pairs to about 10,000 base pairs, about 8,000 base pairs to about 15,000 base pairs, about 9,000 base pairs to about 10,000 base pairs, about 9,000 base pairs to about 15,000 base pairs, or about 10,000 base pairs to about 15,000 base pairs. The length of the nucleic acid molecules to be tagged can be about 500 base pairs, about 1,000 base pairs, about 2,000 base pairs, about 3,000 base pairs, about 4,000 base pairs, about 5,000 base pairs, about 6,000 base pairs, about 7,000 base pairs, about 8,000 base pairs, about 9,000 base pairs, about 10,000 base pairs, or about 15,000 base pairs.

Sequence information from uniquely barcoded dsDNA molecules of varying lengths can be obtained after NGS library preparation and short-read sequencing. Any of the methods of the present disclosure can further comprise phasing the obtained sequences based on their molecular origin as indicated by the unique partition-specific and molecule-specific barcode. The short-read sequencing information can be clustered using partition-specific followed by molecule-specific tags and can be assembled into de novo sequences. The resulting sequences can be phased reconstruction of the original long nucleic acid molecules and can share any degree of homology or similarity with each other. By comparing long sequences that are identical or share any commonality in their classification with each other, the method of the present disclosure can provide a distinct advantage in quantitative analysis for estimating the abundance of different molecules in a pool of parental long molecules.

PCR amplification can be used to generate multiple copies of each parental long nucleic acid molecule with a molecule-specific terminal tag. Amplification can be completed in a single reaction, wherein each sample with a pool of uniquely tagged molecules can be amplified individually. Alternatively, amplification can be completed as a multiplexed reaction, wherein multiple samples, each with a pool of uniquely tagged molecules with a sample-specific sequence shared amongst the pool, can be amplified as a single reaction.

Short-read sequences can be clustered into consensus sequences based on the unique partition-specific and molecule-specific barcode sequences. Consensus sequences can be used for reference mapping and phased into long contigs.

A phased sequence can be utilized to determine the expression of previously unidentified alternative transcripts, for quality control of synthesized long nucleic acid molecules, for identifying the length of repetitive sequences and the like. The methods of the present disclosure can be used to overcome the challenges of obtaining high-quality, long phased DNA sequence.

The present disclosure can contemplate numerical ranges. Where a range of values is provided, it is intended that the ranges include the range endpoints, and each intervening value between the upper and lower limit of that range and any other stated or intervening value in that stated range is encompassed within the disclosure. For example, if a range of 6 to 12 nucleotides is stated, it is intended that 6 nucleotides, 7 nucleotides, 8 nucleotides, 9 nucleotides, 10 nucleotides, 11 nucleotides, and 12 nucleotides are also explicitly disclosed, as well as the range of values greater than or equal to 6 nucleotides and the range of values less than or equal to 12 nucleotides. Additionally, when applicable every sub range and value within the rage is present as if explicitly written out.

The term “about” or “approximately” can mean within an acceptable error range for the particular value as determined by one of ordinary skill in the art, which will depend in part on how the value is measured or determined, e.g., the limitations of the measurement system. For example, “about” can mean within 1, 1.5, 2, 2.5, 3, or more standard deviations. Alternatively, “about” can mean a range of up to 20%, up to 10%, up to 5%, or up to 1% of a given value. Alternatively, with respect to biological systems or processes, the term can mean within an order of magnitude, within 5-fold, or within 2-fold, of a value. Where particular values are described in the application and claims, unless otherwise stated, the term “about” can generally mean within an acceptable error range for the particular value.

As used herein, the term “nucleic acid” or “nucleic acid molecules” can include any form of DNA or RNA, including, for example, genomic DNA; complementary DNA (cDNA), which can be obtained from messenger RNA (mRNA) by reverse transcription or by amplification; DNA molecules produced synthetically or by amplification; cell-free DNA; cell free RNA; mRNA, tRNA and rRNA. Nucleic acid(s) can be derived from chemical synthesis (e.g., solid phase-mediated chemical synthesis), from a biological source (e.g., isolation from any organism), or from processes that involve the manipulation of nucleic acids using molecular biology tools (e.g., cloning, DNA replication, PCR amplification, reverse transcription, or any combination thereof). A nucleic acid can be DNA and/or RNA.

As used herein, the term “sequencing” can refer to determining the order of nucleotides (base sequences) in a nucleic acid sample (e.g., DNA or RNA).

As used herein, the phrases “target nucleotide sequence” or “parental nucleic acid molecule to be sequenced” can refer to a polynucleotide molecule representing a reference (complete) nucleotide sequence of a long target nucleic acid being sequenced, such as the amplification product obtained by amplifying a target nucleic acid or the cDNA produced upon reverse transcription of an RNA target nucleic acid.

The term “oligonucleotide” is used to refer to a nucleic acid that is relatively short, generally shorter than about 200 nucleotides, shorter than about 100 nucleotides, or shorter than about 50 nucleotides. As used herein, the term “oligonucleotide” can refer to a nucleic acid with a length, for example, shorter than about 1,000 nucleotides, shorter than about 900 nucleotides, shorter than about 800 nucleotides, shorter than about 700 nucleotides, shorter than about 600 nucleotides, shorter than about 500 nucleotides, shorter than about 400 nucleotides, shorter than about 300 nucleotides, shorter than about 200 nucleotides, shorter than about 100 nucleotides, or shorter than about 50 nucleotides. An oligonucleotide can range between about 15 nucleotides to about 30 nucleotides, about 20 nucleotides to about 50 nucleotides, about 20 nucleotides to about 100 nucleotides, about 50 nucleotides to about 200 nucleotides, about 50 nucleotides to about 100 nucleotides, about 50 nucleotides to about 150 nucleotides, about 50 nucleotides to about 200 nucleotides, about 100 nucleotides to about 150 nucleotides, about 100 nucleotides to about 200 nucleotides, about 150 nucleotides to about 200 nucleotides. An oligonucleotide can be about 50 nucleotides, about 100 nucleotides, about 150 nucleotides, or about 200 nucleotides. An oligonucleotide can be at least about 15 nucleotides, at least about 20 nucleotides, at least about 25 nucleotides, at least about 30 nucleotides, at least 50 nucleotides, at least about 100 nucleotides, at most about 200 nucleotides, at most about 300 nucleotides, or at most about 500 nucleotides.

As used herein, the term “primer” can refer to an oligonucleotide that is capable of hybridizing (also termed “annealing”) with a nucleic acid and serving as an initiation site for nucleotide (RNA or DNA) polymerization under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer. A primer can be, for example, at least 7 nucleotides long. A primer can range from about 10 to 30 nucleotides or from about 15 to about 30 nucleotides, in length. Primers can also be longer, e.g., about 30 to about 50 nucleotides long. A primer does not necessarily need to be 100% complementary to a template, for example, to be effective. A primer need only be sufficiently complementary in order to hybridize with a template under amplification or sequencing conditions, as appropriate.

A primer can have a length of, for example, 7 nucleotides to 75 nucleotides. A primer can have a length of, for example, at least 7 nucleotides. A primer can have a length of, for example, at most 75 nucleotides. A primer can have a length of, for example, 7 nucleotides to 10 nucleotides, 7 nucleotides to 15 nucleotides, 7 nucleotides to 20 nucleotides, 7 nucleotides to 25 nucleotides, 7 nucleotides to 30 nucleotides, 7 nucleotides to 35 nucleotides, 7 nucleotides to 40 nucleotides, 7 nucleotides to 45 nucleotides, 7 nucleotides to 50 nucleotides, 7 nucleotides to 60 nucleotides, 7 nucleotides to 75 nucleotides, 10 nucleotides to 15 nucleotides, 10 nucleotides to 20 nucleotides, 10 nucleotides to 25 nucleotides, 10 nucleotides to 30 nucleotides, 10 nucleotides to 35 nucleotides, 10 nucleotides to 40 nucleotides, 10 nucleotides to 45 nucleotides, 10 nucleotides to 50 nucleotides, 10 nucleotides to 60 nucleotides, 10 nucleotides to 75 nucleotides, 15 nucleotides to 20 nucleotides, 15 nucleotides to 25 nucleotides, 15 nucleotides to 30 nucleotides, 15 nucleotides to 35 nucleotides, 15 nucleotides to 40 nucleotides, 15 nucleotides to 45 nucleotides, 15 nucleotides to 50 nucleotides, 15 nucleotides to 60 nucleotides, 15 nucleotides to 75 nucleotides, 20 nucleotides to 25 nucleotides, 20 nucleotides to 30 nucleotides, 20 nucleotides to 35 nucleotides, 20 nucleotides to 40 nucleotides, 20 nucleotides to 45 nucleotides, 20 nucleotides to 50 nucleotides, 20 nucleotides to 60 nucleotides, 20 nucleotides to 75 nucleotides, 25 nucleotides to 30 nucleotides, 25 nucleotides to 35 nucleotides, 25 nucleotides to 40 nucleotides, 25 nucleotides to 45 nucleotides, 25 nucleotides to 50 nucleotides, 25 nucleotides to 60 nucleotides, 25 nucleotides to 75 nucleotides, 30 nucleotides to 35 nucleotides, 30 nucleotides to 40 nucleotides, 30 nucleotides to 45 nucleotides, 30 nucleotides to 50 nucleotides, 30 nucleotides to 60 nucleotides, 30 nucleotides to 75 nucleotides, 35 nucleotides to 40 nucleotides, 35 nucleotides to 45 nucleotides, 35 nucleotides to 50 nucleotides, 35 nucleotides to 60 nucleotides, 35 nucleotides to 75 nucleotides, 40 nucleotides to 45 nucleotides, 40 nucleotides to 50 nucleotides, 40 nucleotides to 60 nucleotides, 40 nucleotides to 75 nucleotides, 45 nucleotides to 50 nucleotides, 45 nucleotides to 60 nucleotides, 45 nucleotides to 75 nucleotides, 50 nucleotides to 60 nucleotides, 50 nucleotides to 75 nucleotides, or 60 nucleotides to 75 nucleotides. A primer can have a length of, for example, 7 nucleotides, 10 nucleotides, 15 nucleotides, 20 nucleotides, 25 nucleotides, 30 nucleotides, 35 nucleotides, 40 nucleotides, 45 nucleotides, 50 nucleotides, 60 nucleotides, or 75 nucleotides.

As used herein, the terms “primer site” and “primer binding site” can refer to the segment of a target nucleic acid to which a primer hybridizes.

As used herein, the term “primer pair” can refer to a set of primers including a 5′ “upstream primer” or “forward primer” that can hybridize with the complement of the 5′ end of the nucleic acid sequence to be amplified and a 3′ “downstream primer” or “reverse primer” that can hybridize with the 3′ end of the sequence to be amplified. As will be recognized by those of skill in the art, the terms “upstream” and “downstream” or “forward” and “reverse” are not intended to be limiting, but rather provide illustrative orientation in particular embodiments.

As used herein, the term “amplification” can encompass any manner by which at least a part of one or more target nucleic acid is reproduced, for example in a template-dependent manner. A broad range of techniques can be used to amplify nucleic acid sequences, either linearly or exponentially. Illustrative methods for performing amplification include ligase chain reaction (LCR), ligase detection reaction (LDR), ligation followed by Q-replicase amplification, polymerase chain reaction (PCR), primer extension, strand displacement amplification (SDA), hyperbranched strand displacement amplification, multiple displacement amplification (MDA), nucleic acid strand-based amplification (NASBA), two-step multiplexed amplification, and rolling circle amplification (RCA), including multiplex versions and combinations thereof. Examples of multiplex versions and combinations of amplification procedures include, but are not limited to, oligonucleotide ligation assay (OLA)/PCR, PCR/OLA, LDR/PCR, PCR/PCR/LDR, PCR/LDR, LCR/PCR, and PCR/LCR (also known as combined chain reaction (CCR)), and the like.

Amplification can comprise at least one cycle of the sequential procedures of: denaturing the nucleic acid duplex to separate the strands, annealing at least one primer with complementary or substantially complementary sequences in at least one target nucleic acid; and synthesizing at least one strand of nucleotides in a template-dependent manner using a polymerase. The cycle may or may not be repeated.

As used herein, the term “adjacent,” can refer to two nucleotide sequences in a nucleic acid. “Adjacent” can refer to nucleotide sequences separated by 0 to about 20 nucleotides, 0 to about 50 nucleotides, or in a range of about 1 to about 10 nucleotides, or sequences that are directly about one another.

As used herein, the terms “nucleotide tag”, “molecular tag” and “barcode tag” can refer to a combination of nucleotide sequences (e.g., unique nucleotide sequences) that can be added to a target nucleotide sequence and, in some cases, can serve as a tag. A portion, the entire length, or none of the nucleotide combination that serves as a tag can be a predetermined sequence, or can be determined empirically during sequence data analysis. The molecular tag can include a specific and/or unique nucleotide sequence that encodes information about the amplicon produced when the barcode primer is employed in an amplification reaction. For example, a different tag can be employed to one or more target sequence from each of a number of different samples, such that the barcode nucleotide sequence indicates the sample origin of the resulting amplicons. The molecular tag can also include a shared or universal sequence, which allows for the simultaneous amplification of differently tagged molecules. For example, P5 and P7 Illumina universal primers may be employed. The sequence of a molecular tag can be random, semi-random, fixed, or predetermined.

As used herein, the term “tag” can refer to a short sequence that can be added to a primer, included in a sequence, or otherwise used as label to provide a unique identifier. A sequence identifier can be a unique base sequence of varying but defined length that is used to identify a specific nucleic acid sample. For example, 4 base pair (bp) tags allow 4⁴=256 different tags. A tag can be used to determine the origin of a sample upon further processing. For example, a unique sequence tag can be used to identify the origin and coordinates of the individual sequence in the pool of a complex nucleic acid sequence mixture or amplified library. Multiple tags can be used in the methods of the present disclosure. An example of a tag is a ZIP sequence or GC-rich sequences. A tag can be used to determine the origin of a PCR sample. In the case of combining processed products originating from different nucleic acid samples, the different nucleic acid samples can be identified using different tags.

The tag can be captured on a solid support. The tag can be biotin and be recognized by avidin. An affinity tag can include multiple biotin residues for increased binding to multiple avidin molecules. The tag can also include a functional group such as an azido group or an acetylene group, which enables capture through copper(I) mediated click chemistry (see H. C. Kolb and K. B. Sharpless, Drug Discovery Today, 2003, 8(24), 1128-1137). The tag can include an antigen that can be captured by an antibody bound on a solid support. Examples of tag can include, but are not limited to, His-tag, His6-tag (SEQ ID NO: 3), Calmodulin-tag, CBP, CYD (covalent yet dissociable NorpD peptide). Strep II, FLAG-tag, HA-tag, Myc-tag, S-tag, SBP-tag, Softag-1, Softag-3, V5-tag, Xpress-tag, Isopeptag, SpyTag, B, HPC (heavy chain of protein C) peptide tags, GST, MBP, biotin, biotin carboxyl carrier protein, glutathione-S-transferase-tag, green fluorescent protein-tag, maltose binding protein-tag, Nus-tag, Strep-tag, thioredoxin-tag, and combinations thereof. In some instances, the tagged molecule can be subject to sequencing.

As used herein, the terms “tagging”, “barcoding”, and “encoding reaction” can refer to reactions in which at least one nucleotide tag is added to a target nucleotide sequence. For example, a library of nucleic acid molecules can be tagged with molecule-specific barcodes, for example, by PCR amplification of the nucleic acid library. The PCR primers can insert molecule-specific barcode sequences at the termini of nucleic acid molecules. Alternatively, the barcode segment can be added to the nucleic acid library by ligating the molecule specific barcodes at the termini of nucleic acid molecules using a DNA ligase.

As used herein, the term “tagged target nucleotide sequence” can refer to a nucleotide sequence with an appended nucleotide tag.

As used herein, the term “distributing or proximizing the barcode to different parts of the sequence” can refer to a process or reaction in which a barcode is made proximal (near or adjacent) to a different part of the same nucleic acid molecule it resides on. The barcode can be made proximal through a polymerase-based primed nucleic acid elongation reaction that is facilitated by a nucleic acid priming sequence adjacent to the barcode. The polymerase priming sequence can be a randomer (e.g., 6-20 random bases). There can be many copies of a molecule with a unique single barcode, but each copy can have a different random self-elongation sequence. Therefore, the random priming can collectively translocate, distribute, or proximize the nucleic acid barcode, which can be near or adjacent to the random self-elongation sequence, to all parts of a nucleic acid molecule in an even manner. The copied sequences arising from the random priming events on the same parental long nucleic acid molecule can share the same molecule-specific barcodes.

The polymerase priming sequence can be a randomer having a length of, for example, 6 random bases to 25 random bases. The polymerase priming sequence can be a randomer having a length of, for example, at least 6 random bases. The polymerase priming sequence can be a randomer having a length of, for example, at most 25 random bases. The polymerase priming sequence can be a randomer having a length of, for example, 6 random bases to 8 random bases, 6 random bases to 10 random bases, 6 random bases to 11 random bases, 6 random bases to 12 random bases, 6 random bases to 13 random bases, 6 random bases to 14 random bases, 6 random bases to 15 random bases, 6 random bases to 16 random bases, 6 random bases to 18 random bases, 6 random bases to 20 random bases, 6 random bases to 25 random bases, 8 random bases to 10 random bases, 8 random bases to 11 random bases, 8 random bases to 12 random bases, 8 random bases to 13 random bases, 8 random bases to 14 random bases, 8 random bases to 15 random bases, 8 random bases to 16 random bases, 8 random bases to 18 random bases, 8 random bases to 20 random bases, 8 random bases to 25 random bases, 10 random bases to 11 random bases, 10 random bases to 12 random bases, 10 random bases to 13 random bases, 10 random bases to 14 random bases, 10 random bases to 15 random bases, 10 random bases to 16 random bases, 10 random bases to 18 random bases, 10 random bases to 20 random bases, 10 random bases to 25 random bases, 11 random bases to 12 random bases, 11 random bases to 13 random bases, 11 random bases to 14 random bases, 11 random bases to 15 random bases, 11 random bases to 16 random bases, 11 random bases to 18 random bases, 11 random bases to 20 random bases, 11 random bases to 25 random bases, 12 random bases to 13 random bases, 12 random bases to 14 random bases, 12 random bases to 15 random bases, 12 random bases to 16 random bases, 12 random bases to 18 random bases, 12 random bases to 20 random bases, 12 random bases to 25 random bases, 13 random bases to 14 random bases, 13 random bases to 15 random bases, 13 random bases to 16 random bases, 13 random bases to 18 random bases, 13 random bases to 20 random bases, 13 random bases to 25 random bases, 14 random bases to 15 random bases, 14 random bases to 16 random bases, 14 random bases to 18 random bases, 14 random bases to 20 random bases, 14 random bases to 25 random bases, 15 random bases to 16 random bases, 15 random bases to 18 random bases, 15 random bases to 20 random bases, 15 random bases to 25 random bases, 16 random bases to 18 random bases, 16 random bases to 20 random bases, 16 random bases to 25 random bases, 18 random bases to 20 random bases, 18 random bases to 25 random bases, or 20 random bases to 25 random bases. The polymerase priming sequence can be a randomer having a length of, for example, 6 random bases, 8 random bases, 10 random bases, 11 random bases, 12 random bases, 13 random bases, 14 random bases, 15 random bases, 16 random bases, 18 random bases, 20 random bases, or 25 random bases.

As used herein, the term “elongation-primed single-stranded nucleic acid or ssDNA” can refer to single-stranded nucleic acid or ssDNA molecules with 3′ termini that can function as priming sequences for polymerase-driven DNA polymerization of single-stranded nucleic acid or ssDNA molecules.

As used herein, the term “enrichment PCR” can refer to PCR primer extension that can occur after intramolecular elongation of a nucleotide.

As used herein, the term “clustering” can refer to the comparison of two or more nucleotide sequences based on the presence of short or long stretches of identical or similar nucleotides. Clustering is also referred to using the terms “assembly” or “alignment”.

As used herein, the term “paired end sequencing” can refer to a method based on high throughput sequencing that generates sequencing data from both ends of a nucleic acid molecule.

As used herein, the terms “ligation adapters” or “adapters” can refer to short nucleic acid (e.g., dsDNA) molecules with a length of e.g. about 10 to about 30 bp or from about 10 to about 80 base pairs. An adapter can be appended to a nucleic acid molecule by ligation. An adapter can be appended to a nucleic acid molecule by polymerase chain reaction. Adapters can be composed of two synthetic oligonucleotides, which have nucleotide sequences that can be partially or completely complementary to each other. When mixing the two synthetic oligonucleotides in solution under appropriate conditions, the two synthetic oligonucleotides can anneal to each other to form a double-stranded structure. After annealing, one end of the adapter molecule is designed to be compatible with the end of a nucleic acid fragment and can be ligated thereto. The other end of the adapter can be designed so that it cannot be ligated, but this may not be the case (e.g., double ligated adapters). Adapters can contain other functional features, such as identifiers, recognition sequences for restriction enzymes, and primer binding sections. When containing other functional features, the length of the adapters may increase; the length of the adapters can be controlled and minimized by combining functional features.

The length of an adapter can be about 10 bases or base pairs to about 100 bases or base pairs. The length of an adapter can be at least about 10 bases or base pairs. The length of an adapter can be at most about 100 bases or base pairs. The length of an adapter can be about 10 bases or base pairs to about 20 bases or base pairs, about 10 bases or base pairs to about 30 bases or base pairs, about 10 bases or base pairs to about 40 bases or base pairs, about 10 bases or base pairs to about 50 bases or base pairs, about 10 bases or base pairs to about 60 bases or base pairs, about 10 bases or base pairs to about 70 bases or base pairs, about 10 bases or base pairs to about 80 bases or base pairs, about 10 bases or base pairs to about 90 bases or base pairs, about 10 bases or base pairs to about 100 bases or base pairs, about 20 bases or base pairs to about 30 bases or base pairs, about 20 bases or base pairs to about 40 bases or base pairs, about 20 bases or base pairs to about 50 bases or base pairs, about 20 bases or base pairs to about 60 bases or base pairs, about 20 bases or base pairs to about 70 bases or base pairs, about 20 bases or base pairs to about 80 bases or base pairs, about 20 bases or base pairs to about 90 bases or base pairs, about 20 bases or base pairs to about 100 bases or base pairs, about 30 bases or base pairs to about 40 bases or base pairs, about 30 bases or base pairs to about 50 bases or base pairs, about 30 bases or base pairs to about 60 bases or base pairs, about 30 bases or base pairs to about 70 bases or base pairs, about 30 bases or base pairs to about 80 bases or base pairs, about 30 bases or base pairs to about 90 bases or base pairs, about 30 bases or base pairs to about 100 bases or base pairs, about 40 bases or base pairs to about 50 bases or base pairs, about 40 bases or base pairs to about 60 bases or base pairs, about 40 bases or base pairs to about 70 bases or base pairs, about 40 bases or base pairs to about 80 bases or base pairs, about 40 bases or base pairs to about 90 bases or base pairs, about 40 bases or base pairs to about 100 bases or base pairs, about 50 bases or base pairs to about 60 bases or base pairs, about 50 bases or base pairs to about 70 bases or base pairs, about 50 bases or base pairs to about 80 bases or base pairs, about 50 bases or base pairs to about 90 bases or base pairs, about 50 bases or base pairs to about 100 bases or base pairs, about 60 bases or base pairs to about 70 bases or base pairs, about 60 bases or base pairs to about 80 bases or base pairs, about 60 bases or base pairs to about 90 bases or base pairs, about 60 bases or base pairs to about 100 bases or base pairs, about 70 bases or base pairs to about 80 bases or base pairs, about 70 bases or base pairs to about 90 bases or base pairs, about 70 bases or base pairs to about 100 bases or base pairs, about 80 bases or base pairs to about 90 bases or base pairs, about 80 bases or base pairs to about 100 bases or base pairs, or about 90 bases or base pairs to about 100 bases or base pairs. The length of an adapter can be about 10 bases or base pairs, about 20 bases or base pairs, about 30 bases or base pairs, about 40 bases or base pairs, about 50 bases or base pairs, about 60 bases or base pairs, about 70 bases or base pairs, about 80 bases or base pairs, about 90 bases or base pairs, or about 100 bases or base pairs. An adapter can have a length of, for example, 8 base pairs to 40 base pairs. An adapter can have a length of, for example, at least 8 base pairs. An adapter can have a length of, for example, at most 40 base pairs. An adapter can have a length of, for example, 8 base pairs to 10 base pairs, 8 base pairs to 15 base pairs, 8 base pairs to 20 base pairs, 8 base pairs to 25 base pairs, 8 base pairs to 30 base pairs, 8 base pairs to 35 base pairs, 8 base pairs to 40 base pairs, 10 base pairs to 15 base pairs, 10 base pairs to 20 base pairs, 10 base pairs to 25 base pairs, 10 base pairs to 30 base pairs, 10 base pairs to 35 base pairs, 10 base pairs to 40 base pairs, 15 base pairs to 20 base pairs, 15 base pairs to 25 base pairs, 15 base pairs to 30 base pairs, 15 base pairs to 35 base pairs, 15 base pairs to 40 base pairs, 20 base pairs to 25 base pairs, 20 base pairs to 30 base pairs, 20 base pairs to 35 base pairs, 20 base pairs to 40 base pairs, 25 base pairs to 30 base pairs, 25 base pairs to 35 base pairs, 25 base pairs to 40 base pairs, 30 base pairs to 35 base pairs, 30 base pairs to 40 base pairs, or 35 base pairs to 40 base pairs. An adapter can have a length of, for example, 8 base pairs, 10 base pairs, 15 base pairs, 20 base pairs, 25 base pairs, 30 base pairs, 35 base pairs, or 40 base pairs.

As used herein, the term “terminal adapters” can refer to nucleic acid (e.g., ssDNA) molecules with, e.g. about 20 to 200 bases or 20 to 100 bases. A terminal adapter can have a length of, for example, 20 bases to 100 bases. A terminal adapter can have a length of, for example, at least 20 bases. A terminal adapter can have a length of, for example, at most 100 bases. A terminal adapter can have a length of about, for example, 20 bases to 30 bases, 20 bases to 40 bases, 20 bases to 50 bases, 20 bases to 60 bases, 20 bases to 70 bases, 20 bases to 80 bases, 20 bases to 100 bases, 30 bases to 40 bases, 30 bases to 50 bases, 30 bases to 60 bases, 30 bases to 70 bases, 30 bases to 80 bases, 30 bases to 100 bases, 40 bases to 50 bases, 40 bases to 60 bases, 40 bases to 70 bases, 40 bases to 80 bases, 40 bases to 100 bases, 50 bases to 60 bases, 50 bases to 70 bases, 50 bases to 80 bases, 50 bases to 100 bases, 60 bases to 70 bases, 60 bases to 80 bases, 60 bases to 100 bases, 70 bases to 80 bases, 70 bases to 100 bases, or 80 bases to 100 bases. A terminal adapter can have a length of, for example, 20 bases, 30 bases, 40 bases, 50 bases, 60 bases, 70 bases, 80 bases, or 100 bases. Terminal adapters can be designed to be used as primers in conjunction with a polymerase to append nucleic acid molecules with specific sequences, including molecule-specific barcodes, sequences for downstream amplifications, and sequences used for NGS sequencing. Terminal adapters can contain self-elongation sequences for extending and copying sequences that can be internal to the nucleic acid molecule.

As used herein, the term “sequencing adapters” can refer to nucleic acid molecules (e.g., single-stranded DNA (ssDNA)) with, e.g., about 20 to 80 bases. A sequencing adapter can have a length of, for example, 20 bases to 80 bases. A sequencing adapter can have a length of, for example, at least 20 bases. A sequencing adapter can have a length of, for example, at most 80 bases. A sequencing adapter can have a length of, for example, 20 bases to 30 bases, 20 bases to 40 bases, 20 bases to 50 bases, 20 bases to 60 bases, 20 bases to 70 bases, 20 bases to 80 bases, 30 bases to 40 bases, 30 bases to 50 bases, 30 bases to 60 bases, 30 bases to 70 bases, 30 bases to 80 bases, 40 bases to 50 bases, 40 bases to 60 bases, 40 bases to 70 bases, 40 bases to 80 bases, 50 bases to 60 bases, 50 bases to 70 bases, 50 bases to 80 bases, 60 bases to 70 bases, 60 bases to 80 bases, or 70 bases to 80 bases. A sequencing adapter can have a length of, for example, 20 bases, 30 bases, 40 bases, 50 bases, 60 bases, 70 bases, or 80 bases. Sequencing adapters can be universal sequences that can be used in high throughput sequencing. For example, sequencing adapters can contain universal sequences used by high throughput sequencers to capture nucleic acid libraries and generate sequencing clusters (e.g. P5 and P7 sequences), and to generate short reads information (e.g. Read 1 and Read 2 sequences) and sample index information (e.g. P5, P7 and Read 2 sequences).

The length of a sequencing adapter can be about 10 bases or base pairs to about 100 bases or base pairs. The length of a sequencing adapter can be at least about 10 bases or base pairs. The length of a sequencing adapter can be at most about 100 bases or base pairs. The length of a sequencing adapter can be about 10 bases or base pairs to about 20 bases or base pairs, about 10 bases or base pairs to about 30 bases or base pairs, about 10 bases or base pairs to about 40 bases or base pairs, about 10 bases or base pairs to about 50 bases or base pairs, about 10 bases or base pairs to about 60 bases or base pairs, about 10 bases or base pairs to about 70 bases or base pairs, about 10 bases or base pairs to about 80 bases or base pairs, about 10 bases or base pairs to about 90 bases or base pairs, about 10 bases or base pairs to about 100 bases or base pairs, about 20 bases or base pairs to about 30 bases or base pairs, about 20 bases or base pairs to about 40 bases or base pairs, about 20 bases or base pairs to about 50 bases or base pairs, about 20 bases or base pairs to about 60 bases or base pairs, about 20 bases or base pairs to about 70 bases or base pairs, about 20 bases or base pairs to about 80 bases or base pairs, about 20 bases or base pairs to about 90 bases or base pairs, about 20 bases or base pairs to about 100 bases or base pairs, about 30 bases or base pairs to about 40 bases or base pairs, about 30 bases or base pairs to about 50 bases or base pairs, about 30 bases or base pairs to about 60 bases or base pairs, about 30 bases or base pairs to about 70 bases or base pairs, about 30 bases or base pairs to about 80 bases or base pairs, about 30 bases or base pairs to about 90 bases or base pairs, about 30 bases or base pairs to about 100 bases or base pairs, about 40 bases or base pairs to about 50 bases or base pairs, about 40 bases or base pairs to about 60 bases or base pairs, about 40 bases or base pairs to about 70 bases or base pairs, about 40 bases or base pairs to about 80 bases or base pairs, about 40 bases or base pairs to about 90 bases or base pairs, about 40 bases or base pairs to about 100 bases or base pairs, about 50 bases or base pairs to about 60 bases or base pairs, about 50 bases or base pairs to about 70 bases or base pairs, about 50 bases or base pairs to about 80 bases or base pairs, about 50 bases or base pairs to about 90 bases or base pairs, about 50 bases or base pairs to about 100 bases or base pairs, about 60 bases or base pairs to about 70 bases or base pairs, about 60 bases or base pairs to about 80 bases or base pairs, about 60 bases or base pairs to about 90 bases or base pairs, about 60 bases or base pairs to about 100 bases or base pairs, about 70 bases or base pairs to about 80 bases or base pairs, about 70 bases or base pairs to about 90 bases or base pairs, about 70 bases or base pairs to about 100 bases or base pairs, about 80 bases or base pairs to about 90 bases or base pairs, about 80 bases or base pairs to about 100 bases or base pairs, or about 90 bases or base pairs to about 100 bases or base pairs. The length of a sequencing adapter can be about 10 bases or base pairs, about 20 bases or base pairs, about 30 bases or base pairs, about 40 bases or base pairs, about 50 bases or base pairs, about 60 bases or base pairs, about 70 bases or base pairs, about 80 bases or base pairs, about 90 bases or base pairs, or about 100 bases or base pairs.

As used herein, the term “covers” can mean that an overlapping group of polynucleotide sequences can be assembled into a contiguous consensus sequence that can span and accurately represents the complete sequence of the parental long nucleic acid molecule being sequenced.

As used herein, the term “coverage-bias” can refer to a non-random distribution of sequence reads covering a longer parental sequence. Lack of even coverage or representation of the parental sequence can occur due to non-random fragmentation and/or site-preferential restriction enzyme digestion. Other bias-inducing methods include intermolecular ligation, which can be limited due to length constraints in the double-stranded DNA (dsDNA) molecule being circularized. Barcode pairing can improve assembly lengths. Reads associated with two distinct barcodes can be aligned to the reference genome. Individually, each group of reads assembles into a contiguous sequence (“contig”) that can be several kilobases in length. Barcode pairing merges the groups, increasing and smoothing coverage across the region to allow assembly of the full 10-kb target sequence. Length histograms of the contigs assembled from genomic reads (minimum length of about 1000 base pairs (bp)) from the reference genome and the sample can be compared.

A population of approximately 10⁰, approximately 10¹, approximately 10², approximately 10³, approximately 10⁴, approximately 10⁵, approximately 10⁶, approximately 10⁷, approximately 10⁸, or approximately 10⁹, nucleic acid molecules in the complex mixture can be used in any of the methods of the present disclosure.

As used herein, the term “phasing” can refer to the determination of a single-molecule origin of sequencing data. For example, phasing can be the ability to cluster nucleic acid sequencing reactions, which generate short stretches of sequencing data (short reads), into longer stretches of nucleic acid sequence information to decipher the sequence of a parental long nucleic acid molecule. Phasing can involve identifying a collection of sequencing reactions (short reads) that span the sequence of a single longer nucleic acid molecule, and accurately reconstructing the sequence of the single long DNA/RNA molecule (long read) from the shorter DNA sequencing reactions (short reads). Phase information can be used to understand gene expression patterns for genetic disease research through the phased sequencing of, for example, human DNA, bacterial DNA and viral DNA. Phasing can be generated through laboratory-based experimental methods, or it can be estimated with computational and statistical approaches. A mixture of nucleic acid molecules from any source can be tagged. The nucleic acid mixture can have any degree of homology, including alleles of a gene within an cell, different versions of a gene within an organism (somatically mutated variants), different versions of a gene within a population of organisms, splice variants, homologous genes, heterologous genes, somatically mutated variants of a gene, duplicated genes and variants of synthetic genes, gene libraries made in a DNA synthesis process or any combination thereof.

As used herein, the term “standard NGS library preparation” can be used to depict a high quality, comprehensive sequencing library preparation. Standard NGS library preparation can be used in NGS methods that employ short read library sample preparation, such as whole-genome sequencing, targeted DNA sequencing, whole-transcriptome sequencing, and targeted RNA sequencing.

### EXAMPLES

The following specific examples are illustrative and non-limiting. The examples described herein reference and provide non-limiting support to the various embodiments described in the preceding sections.

### Example 1: Sequence-Dependent Tagging of RNA Molecules from Single Cells

A single cell suspension was obtained and co-flowed with microparticles functionalized with oligonucleotides containing partition-specific and barcode-specific barcodes to form aqueous droplets that contain one or zero cells and one or zero microparticles in each droplet (see FIG. 4). Each microparticle contained a plurality of terminal tagging adapters comprising a sequencing adapter, a universal PCR sequence, a partition-specific barcode, a molecule-specific barcode, and a poly-thymine sequence. The plurality of tagging adapters on each microparticle shared the same partition-specific barcode that is unique to that microparticle but a different molecule-specific barcode. The microparticle was suspended in lysis buffer to aid in cell lysis and the release of nucleic acid content once the aqueous droplets containing microparticles and single cells were formed. In addition, reverse transcriptase with a terminal transferase activity was included in the aqueous solution during droplet formation, and mRNA molecules were reverse transcribed inside the aqueous partition.

Alternatively, terminal tagging adapters comprising a sequencing adapter, a universal PCR sequence, a partition-specific barcode, a molecule-specific barcode, and a gene-specific sequence were used to selectively reverse transcribe specific RNA molecules from the nucleic acid content inside the aqueous partition.

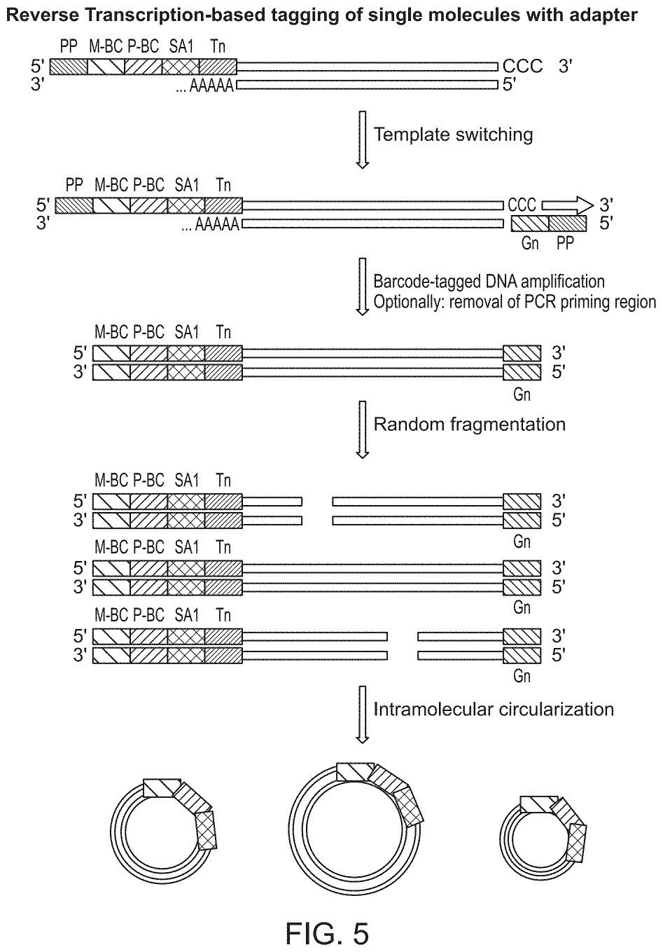

Once reverse transcription reached completion, the aqueous emulsions were broken and the nucleic acid contents from all the aqueous solution were pooled (see FIG. 5). For the complementary DNA (cDNA) that was reverse transcribed to completion, i.e. the reverse transcriptase reaches the 5′ terminal end of the mRNA molecule, the reverse transcriptase with a terminal transferase activity added 2-5 cytosines to 3′ terminal end of the cDNA. The short cystosine repeat was used to anneal a second terminal tag comprising a universal PCR sequence and a short poly-guanine sequence, and the sequence of the second terminal tag was copied onto the 3′ terminal end of the cDNA, thereby forming a mixture of doubly-tagged DNA molecule.

The mixture of cDNA molecules can have any degree of homology. Each of the cDNA molecules in the mixture contained a partition-specific barcode that it shares with other cDNA molecules reverse transcribed within the same partition, as well as a unique molecule-specific barcode. Each of the cDNA molecules in the mixture was then amplified using the universal PCR sequence present on the terminal tags, thereby obtaining a mixture of barcode-tagged double-stranded DNA molecules with many identical copies of the original pool of DNA molecules (see FIG. 5). The amplification of the barcode-tagged DNA molecules was conducted with an uracil-tolerance polymerase and an uracil-containing primer of the universal PCR sequence. Subsequently, the universal PCR priming region was removed by enzymatically digesting the amplified barcode-tagged DNA molecules with a combination of uracil-DNA Glycosylase and an endonuclease to remove the apurinic/apyrimidinic site.

The mixture of amplified barcode-tagged DNA molecules was subjected to enzymatic fragmentation, such that on average each long DNA molecule was cleaved once. A mixture of DNA molecules that contained the 5′ barcode terminal tag, 3′ terminal tag, both 5′ barcode terminal tag and the 3′ terminal tag, or no tag at all was obtained (see FIG. 5). In addition, it was expected that the fragmentation sites are random. Since each uniquely barcoded molecule has many identical copies prior to fragmentation, and that the fragmentation locations are random, the different copies of the uniquely barcoded molecules share the same partition-specific and molecule-specific barcode but a different 3′ end that is generated by fragmentation. Collectively, the locations of the 3′ ends of the pool of uniquely barcoded molecules spanned the entire length of the original barcoded molecule. The fragments were also subjected to enzymatic end-repair to produce blunt ends.

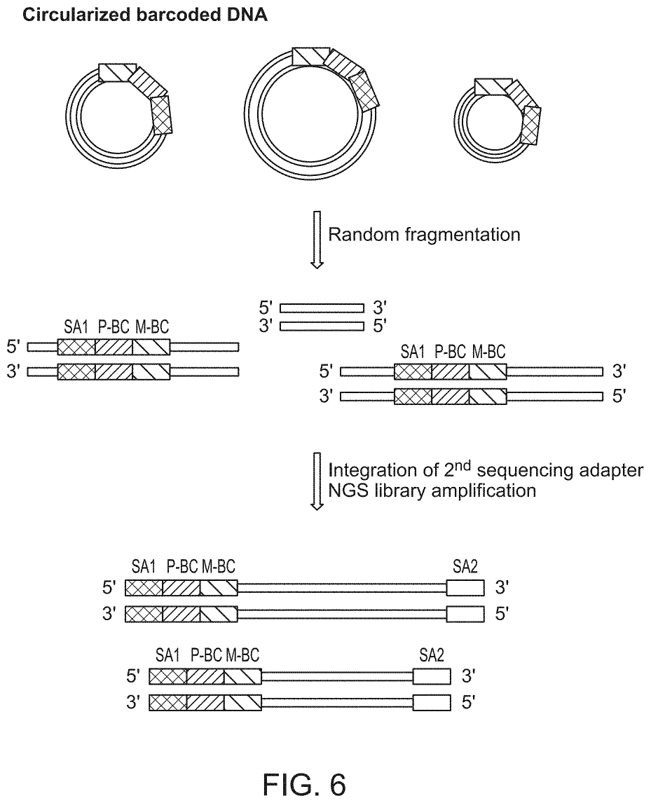

After fragmentation and end-repair, the amplified and barcode-tagged DNA fragments underwent circularization, or intramolecular ligation. Since the 3′ end of the fragments were randomly generated, the intramolecular ligation distributed the partition-specific and molecule-specific barcodes to various locations throughout the barcode-tagged DNA molecules (see FIG. 5 and FIG. 6). The circularized barcode-tagged DNA fragments were subjected to a second fragmentation to linearize the molecules and to produce an available terminal end for attaching a second sequencing adapter. The barcode-tagged DNA fragments with dual-end sequencing adapters were then amplified, size selected, and sequenced.

The short-read sequences were clustered using the partition-specific and molecule-specific barcodes and assembled into contiguous regions of the original molecules using de novo assembly from the short-read sequences. Optionally, the assembled contigs of the original molecules were used to compare with reference sequence of the molecules to establish phasing information from the sample. Quantitative analysis of the de novo assembly and reference mapping were used to characterize the long DNA molecules.

### Example 2

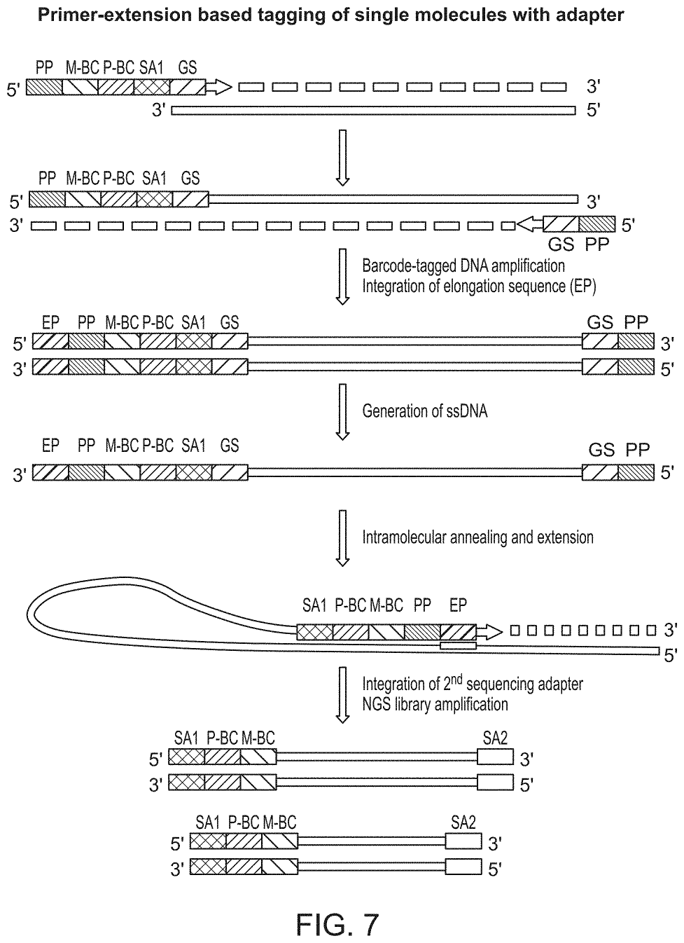

Similar to the method described in Example 1, a single cell suspension was obtained and co-flowed with microparticles functionalized with oligonucleotides containing partition-specific and barcode-specific barcodes to form aqueous droplets that contained one or zero cells and one or zero microparticles in each droplet. Each microparticle contained a plurality of terminal tagging adapters comprising a sequencing adapter, a universal PCR sequence, a partition-specific barcode, a molecule-specific barcode, and a gene-specific sequence. The plurality of tagging adapters on each microparticle shared the same partition-specific barcode that is unique to that microparticle but different molecule-specific barcode. The microparticle was suspended in lysis buffer to aid in cell lysis and the release of nucleic acid content once the aqueous droplets containing microparticles and single cells were formed. DNA polymerase was included in the aqueous solution during droplet formation, and genomic DNA molecules were copied inside the aqueous partition using the gene-specific sequence in the terminal tag as the priming site (see FIG. 7). Optionally, a rare-cutting restriction enzyme was included to aid primer access to the genomic DNA molecules.

Alternatively, terminal tagging adapters comprising a sequencing adapter, a universal PCR sequence, a partition-specific barcode, a molecule-specific barcode, and a random sequence were used to perform sequence-independent tagging of genomic DNA molecules from the nucleic acid content inside the aqueous partition.

Once the DNA molecules were barcoded, the aqueous emulsions were broken and the nucleic acid content from all the aqueous solution were pooled (see FIG. 7). A second terminal tag comprising a universal PCR sequence and a gene-specific sequence downstream of the gene-specific sequence at the barcode-tagging adapter was used to form a mixture of doubly-tagged DNA molecule. Alternatively, the tagged DNA molecules were fragmented and blunted for the purpose to ligating a second terminal tag comprising a universal PCR sequence in a sequence-independent manner.

Each of the DNA molecules in the mixture contained a partition-specific barcode that it shared with other DNA molecules synthesized within the same partition, as well as a unique molecule-specific barcode. Each of the DNA molecules in the mixture was then amplified using the universal PCR sequence present on the terminal tags, thereby obtaining a mixture of barcode-tagged double-stranded DNA molecules with many identical copies of the original pool of DNA molecules (see FIG. 7). Additionally, elongation sequences that are complementary to sequences internal to the barcode-tagged DNA molecules were appended to the terminal ends that contained the partition-specific and the molecule-specific barcode. During amplification, the uniquely barcoded molecules were replicated into a plurality of identical copies, and each replicate of the uniquely barcoded molecules were appended with a different elongation sequence. Collectively, the elongation sequences spanned the entire length of the original barcoded molecule or only specific regions of interest by design. The double-stranded barcode-tagged DNA with appended elongation sequences were denatured, generating a pool of uniquely tagged molecules with the elongation sequence as well as the barcode sequences on the 3′ terminal end.

Alternatively, the single-stranded barcode-tagged DNA molecules were generated from their double-stranded counter parts by enzymatic degradation, e.g via Lambda Exonuclease of a phosphorylated strand, specifically degrading one strand of the uniquely barcoded DNA molecules to obtain a pool of uniquely barcoded and elongation-primed single-stranded DNA molecules.

The amplified and barcode-tagged DNA fragments underwent intramolecular annealing and extension, or elongation, using the elongation sequences on the 3′ terminal end which is complementary to an internal region of the same molecule (see FIG. 7). Since a plurality of elongation sequences were used, the intramolecular elongation distributes the partition-specific and molecule-specific barcodes to various locations throughout the barcode-tagged DNA molecules. Alternatively, the elongation sequence that was appended during amplification of the uniquely tagged DNA molecules were a random sequence, such that the intramolecular elongation occurred in a sequence-independent manner, and the barcode distribution to various locations throughout the barcode-tagged DNA molecules occurred in a sequence-independent manner.

Lastly, a second sequencing adapter was integrated onto the elongated barcode-tagged DNA molecule using PCR primer extension with oligonucleotides comprising the second sequence adapter and gene-specific sequences that are downstream of the elongation sites. The barcode-tagged DNA fragments with dual-end sequencing adapter were then amplified, size selected, and sequenced.

The short-read sequences were clustered using the partition-specific and molecule-specific barcodes and assembled into contiguous or discontiguous regions of the original molecules using de novo assembly from the short-read sequences. Optionally, the assembled contigs of the original molecules were used to compare with reference sequence of the molecules to establish phasing information from the sample. Quantitative analysis of the de novo assembly and reference mapping were used to characterize the long DNA molecules.

### Example 3

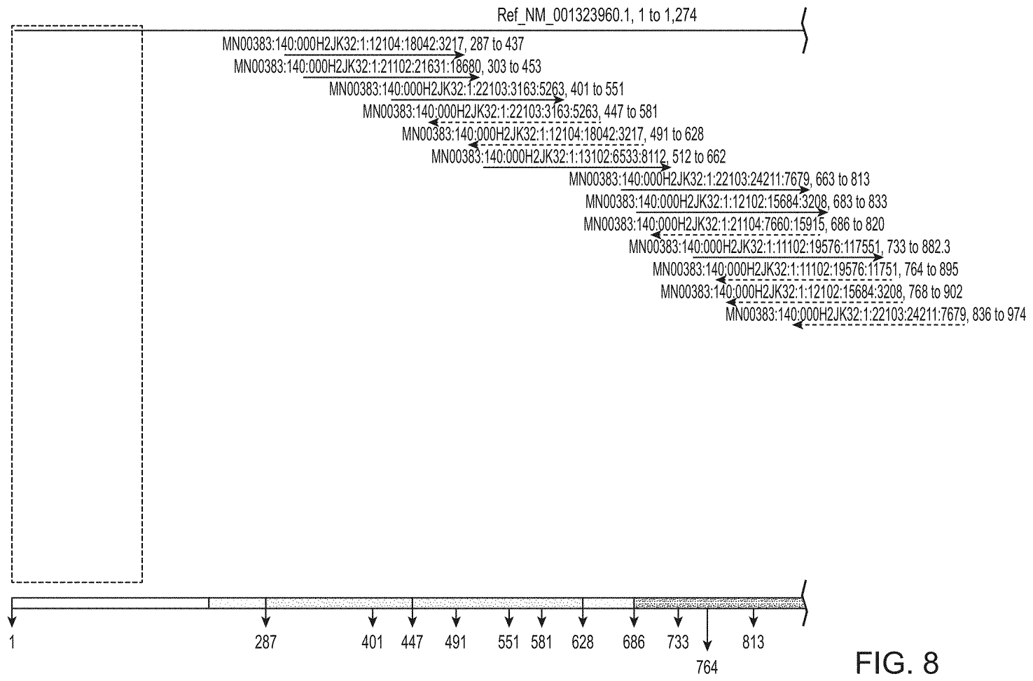

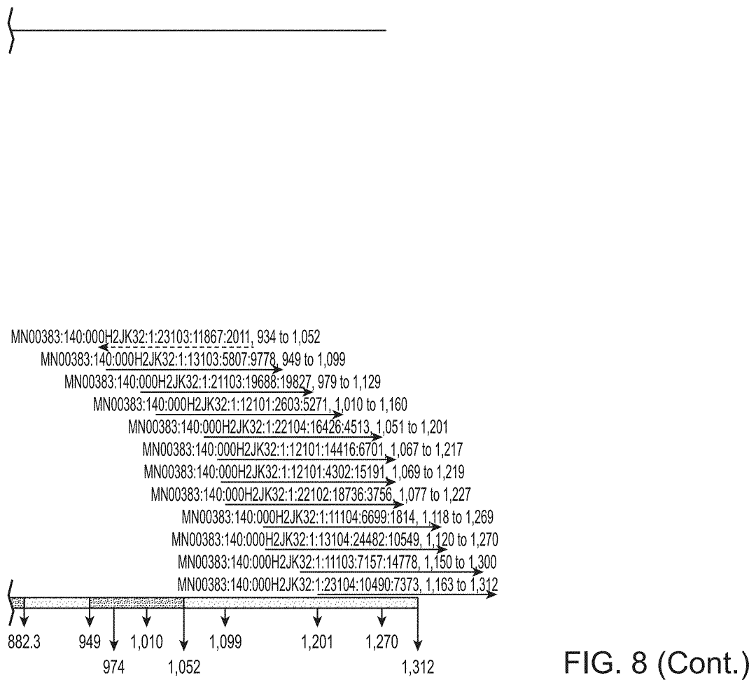

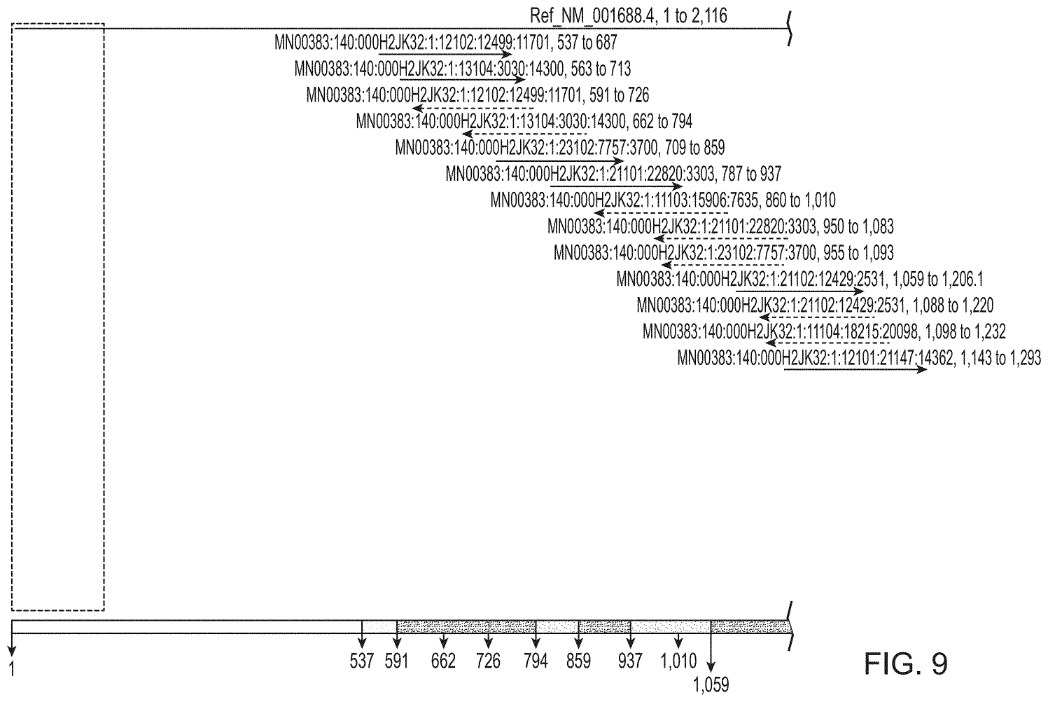

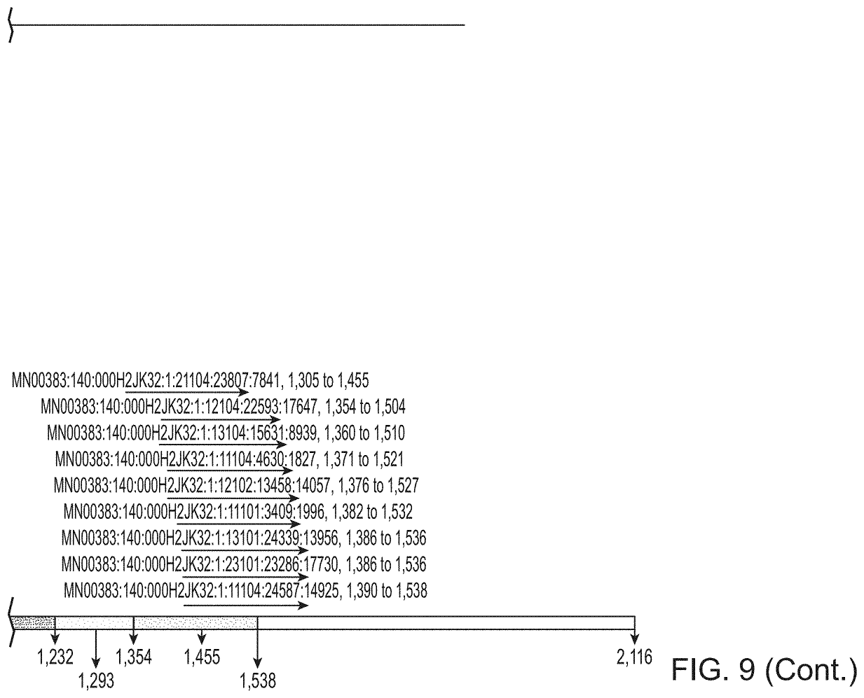

Molecular and cell barcodes were appended at the 5′ end and the 3′ end, respectively, of complementary DNA (cDNA) molecules. After sequencing, the short reads were clustered using the appended molecular barcode sequences and assembled into synthetic long read (SLR) contigs. For each molecular barcode, the assembled synthetic long read contigs were mapped to reference databases and identified (see TABLE 1). Using cell barcodes, synthetic long reads with different molecular barcodes originating from the same cell or partition were grouped together to provide insight on differential expressions pattern from cell to cell. See FIG. 8 and FIG. 9.

TABLE 1

| | SEQ | | SEQ | Short | | | | |
|---|---|---|---|---|---|---|---|---|
| Molecular | ID | Cell | ID | read | SLR | Accession | | |
| barcode | NO: | barcode | NO: | count | length | Number | Description | Length |
| TCAATA | 4 | CATAT | 17 | 27 | 881 | NM_006098.4 | Homo | 1125 |
| CAGTTA | | GGCAG | | | | (see SEQ ID | sapiens | |
| | | TCGATT | | | | NO: 32) | receptor for | |
| | | | | | | | activated C | |
| | | | | | | | kinase 1 | |
| | | | | | | | (RACK1), | |
| | | | | | | | mRNA | |
| AACAGT | 5 | GGTGC | 18 | 18 | 833 | BC067787.1 | Homo | 852 |
| ACCTAG | | GTAGT | | | | (see SEQ ID | sapiens | |
| | | AGCCG | | | | NO: 33) | eukaryotic | |
| | | A | | | | | translation | |
| | | | | | | | elongation | |
| | | | | | | | factor 1 beta | |
| | | | | | | | 2, mRNA | |
| TACCTA | 6 | CCTTCG | 19 | 30 | 826 | BC107711.1 | Homo | 1325 |
| ACCCGC | | ACAAC | | | | (see SEQ ID | sapiens | |
| | | ACCCG | | | | NO: 34) | ribosomal | |
| | | | | | | | protein L3, | |
| | | | | | | | mRNA | |
| CGAACA | 7 | CACCA | 20 | 26 | 829 | NR_073024.1 | Homo | 1186 |
| ATCGAC | | GGTCG | | | | (see SEQ ID | sapiens | |
| | | GCGCT | | | | NO: 35) | ribosomal | |
| | | A | | | | | protein L13a | |
| | | | | | | | (RPL13A), | |
| | | | | | | | transcript | |
| | | | | | | | variant 3, | |
| | | | | | | | non-coding | |
| | | | | | | | RNA | |
| ATAGTA | 8 | ACATA | 21 | 38 | 807 | NM_000968.3 | Homo | 1458 |
| GCGCTT | | CGAGA | | | | (see SEQ ID | sapiens | |
| | | GGTAC | | | | NO: 36) | ribosomal | |
| | | C | | | | | protein L4 | |
| | | | | | | | (RPL4), | |
| | | | | | | | mRNA | |
| GATCAG | 9 | ACATC | 22 | 36 | 819 | NM_00117277 | Homo | 4565 |
| CATATC | | AGCAC | | | | 3.1 (see SEQ | sapiens zinc | |
| | | GGCCA | | | | ID NO: 37) | finger protein | |
| | | T | | | | | 548 | |
| | | | | | | | (ZNF548), | |
| | | | | | | | transcript | |
| | | | | | | | variant 1, | |
| | | | | | | | mRNA | |
| AAAGCG | 10 | AGTGT | 23 | 32 | 755 | XM_00524734 | PREDICTED: | 1681 |
| GACGAA | | CATCT | | | | 7.4 (see SEQ | Homo | |
| | | AACTT | | | | ID NO: 38) | sapiens G | |
| | | C | | | | | protein- | |
| | | | | | | | coupled | |
| | | | | | | | receptor 160 | |
| | | | | | | | (GPR160), | |
| | | | | | | | transcript | |
| | | | | | | | variant X6, | |
| | | | | | | | mRNA | |
| CGCACC | 11 | CAGCT | 24 | 20 | 793 | NM_000969.5 | Homo | 1028 |
| GACCCC | | AAAGA | | | | (see SEQ ID | sapiens | |
| | | CAAGC | | | | NO: 39) | ribosomal | |
| | | C | | | | | protein L5 | |
| | | | | | | | (RPL5), | |
| | | | | | | | transcript | |
| | | | | | | | variant 1, | |
| | | | | | | | mRNA | |
| GCTTCC | 12 | GTGCA | 25 | 28 | 764 | NM_00132396 | Homo | 1274 |
| TTCTGA | | TAGTC | | | | 0.1 (see SEQ | sapiens | |
| | | AGATA | | | | ID NO: 30) | RANBP2- | |
| | | A | | | | | type and | |
| | | | | | | | C3HC4-type | |
| | | | | | | | zinc finger | |
| | | | | | | | containing 1 | |
| | | | | | | | (RBCK1), | |
| | | | | | | | transcript | |
| | | | | | | | variant 5, | |
| | | | | | | | mRNA | |
| TGACTC | 13 | TAGTT | 26 | 26 | 718 | NM_003352.4 | Homo | 1527 |
| CTAAAC | | GGGTT | | | | (see SEQ ID | sapiens small | |
| | | ACCAG | | | | NO: 40) | ubiquitin-like | |
| | | T | | | | | modifier 1 | |
| | | | | | | | (SUMO1), | |
| | | | | | | | transcript | |
| | | | | | | | variant 1, | |
| | | | | | | | mRNA | |
| ACATTA | 14 | GACGT | 27 | 26 | 522 | NM_002869.4 | Homo | 3419 |
| CTGGAC | | TACAT | | | | (see SEQ ID | sapiens | |
| | | CGGTT | | | | NO: 41) | RAB6A, | |
| | | A | | | | | member RAS | |
| | | | | | | | oncogene | |
| | | | | | | | family | |
| | | | | | | | (RAB6A), | |
| | | | | | | | transcript | |
| | | | | | | | variant 1, | |
| | | | | | | | mRNA | |
| AAGAGA | 15 | CGCTTC | 28 | 36 | 725 | NM_016275.4 | Homo | 3527 |
| TCGTAA | | ACACA | | | | (see SEQ ID | sapiens | |
| | | ACGTT | | | | NO: 42) | selenoprotein | |
| | | | | | | | T | |
| | | | | | | | (SELENOT), | |
| | | | | | | | mRNA | |
| GTCAGA | 16 | TTCGGT | 29 | 20 | 753 | NM_001688.4 | Homo | 2116 |
| AGCACT | | CGTCTC | | | | (see SEQ ID | sapiens ATP | |
| | | CATC | | | | NO: 31) | synthase | |
| | | | | | | | peripheral | |
| | | | | | | | stalk- | |
| | | | | | | | membrane | |
| | | | | | | | subunit b | |
| | | | | | | | (ATP5PB), | |
| | | | | | | | mRNA | |

While a number of exemplary aspects and embodiments have been discussed above, it should be understood that the detailed description and drawings are given by way of illustration only, and that various changes and modifications based on this detailed description are encompassed by and fall within the spirit and scope of the present disclosure. It is therefore intended that the following appended claims and claims hereafter introduced are interpreted to include all such modifications, permutations, additions and sub-combinations thereof as are within the true spirit and scope of the present disclosure.

Other limitations will become apparent to those of skill in the art upon a reading of the specification and a study of the drawings. It is to be understood that the methods and compositions described herein are not limited to the particular methodology, protocols, constructs, and reagents described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

All literature and similar materials cited in this application, including, but not limited to, patents, patent applications, NCBI numbers, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

### EMBODIMENTS

1. A method for tagging single nucleic acid molecules for single-cell synthetic long-read (SLR) DNA sequencing or RNA sequencing, the method comprising:
   (a) encapsulating single cells into individual partitions and extracting its nucleic acid content inside each partition;
   (b) tagging the nucleic acid molecules inside each partition with terminal adapters comprising partition-specific barcodes and unique molecule-specific barcodes, thereby obtaining a pool of uniquely barcoded nucleic acid molecules that share the same partition-specific barcode inside each partition;
   (c) providing a plurality of clonal nucleic acid molecules each having the same partition-specific and molecule-specific barcodes at the terminal ends;
   (d) for each nucleic acid molecule, fragmenting the nucleic acid at a random location inside the molecule;
   (e) for each copy of the barcoded nucleic acid molecule, joining the terminal barcoded end with the end generated by random fragmentation and circularizing the molecule via intramolecular ligation;
   (f) for each nucleic acid molecule, sequencing the partition-specific barcode, the molecule-specific barcode, and the internal sequence of the molecule up to and including the end generated by random fragmentation;
   (g) clustering the sequencing data by the molecule-specific barcodes and assembling synthetic long read sequencing data from each barcode cluster for each molecule from the plurality of shorter internal sequences of the nucleic acid molecule;
   (h) clustering the synthetic long-read sequencing data by the cell-specific barcodes to generate cell-specific long-read sequencing data; and
   (i) differentiating between distinct phases, i.e. molecular variants, of highly homologous molecules.
2. The method of Embodiment 1, wherein the method is performed with a plurality of clonal nucleic acid populations each having a different molecule-specific barcodes attached thereto, and a separate sequence is assembled in (g) for each of the molecule-specific barcode.
3. A method for tagging single nucleic acid molecules for single-cell synthetic long-read (SLR) DNA sequencing or RNA sequencing, the method comprising:
   (a) encapsulating single cells into individual partitions and extracting its nucleic acid content inside each partition;
   (b) tagging the nucleic acid molecules inside each partition with partition-specific barcodes on one terminal end;
   (c) tagging the nucleic acid molecules with unique molecule-specific barcodes on the opposing terminal end, thereby obtaining a pool of uniquely barcoded nucleic acid molecules;
   (d) providing a plurality of clonal nucleic acid molecules each having the same partition-specific and molecule-specific barcodes at the terminal ends;
   (e) for each nucleic acid molecule, fragmenting the nucleic acid at a random location inside the molecule;
   (f) for each nucleic acid molecule, joining the terminal end with the molecule-specific barcodes and the end generated by random fragmentation and circularizing the molecule via intramolecular ligation;
   (g) for each nucleic acid molecule, sequencing the partition-specific barcode;
   (h) for each nucleic acid molecule, sequencing the molecule-specific barcode and the internal sequence of the molecule up to and including the end generated by random fragmentation;
   (i) assembling the sequence of the nucleic acid molecule from the plurality of internal sequences of the nucleic acid molecule; and
   (j) differentiating between distinct phases, i.e. molecular variants, of highly homologous molecules.
4. The method of Embodiment 3, wherein the method is performed with a plurality of clonal nucleic acid populations each having a different molecule-specific barcodes attached thereto, and a separate sequence is assembled in (i) for each of the molecule-specific barcode.
5. A method for tagging single nucleic acid molecules for single-cell synthetic long-read (SLR) DNA sequencing or RNA sequencing, the method comprising:
   (a) encapsulating single cells into individual partitions and extracting its nucleic acid content inside each partition;
   (b) tagging the nucleic acid molecules inside each partition with partition-specific barcodes on one terminal end;
   (c) tagging the nucleic acid molecules with unique molecule-specific barcodes on the opposing terminal end, thereby obtaining a pool of uniquely barcoded nucleic acid molecules;
   (d) providing a plurality of clonal nucleic acid molecules each having the same partition-specific and molecule-specific barcodes at the terminal ends;
   (e) for each nucleic acid molecule, joining the terminal end with the partition-specific barcode and the terminal end with the molecule-specific barcode and circularizing the molecule via intramolecular ligation;
   (f) for each nucleic acid molecule, sequencing the partition-specific barcode and the molecule-specific barcode;
   (g) pairing the molecule-specific barcode with the partition-specific barcode from the plurality of barcode sequences; and
   (h) differentiating between the sequences of nucleic acid molecules from different partitions.
6. The method of Embodiment 5, wherein the method is performed with a plurality of clonal nucleic acid populations each having a different molecule-specific barcodes attached thereto, and a separate pairing is established in (g) for each of the molecule-specific barcode.
7. A method for tagging single nucleic acid molecules for single-cell synthetic long-read (SLR) DNA sequencing or RNA sequencing, the method comprising:
   (a) encapsulating single cells into individual partitions and extracting its nucleic acid content inside each partition;
   (b) tagging the nucleic acid molecules inside each partition with terminal adapters comprising partition-specific barcodes and unique molecule-specific barcodes, thereby obtaining a pool of uniquely barcoded DNA molecules;
   (c) providing a plurality of clonal nucleic acid molecules each having the same partition-specific and molecule-specific barcodes at the terminal ends;
   (d) appending the terminal end containing barcodes with an elongation sequence that is also internal to the long nucleic acid molecule;
   (e) for each nucleic acid molecule, denaturing and obtaining single-stranded nucleic acids with the elongation sequence on the 3′ terminal end for intramolecular priming;
   (f) for each nucleic acid molecule, annealing the 3′ terminal end with the elongation sequence at an internal position intramolecularly and extending the molecule;
   (g) for each nucleic acid molecule, sequencing the partition-specific barcode, the molecule-specific barcode, and the internal sequences downstream of the elongation sequence;
   (h) assembling the sequence of the nucleic acid molecule from the plurality of internal sequences of the nucleic acid molecule; and
   (i) differentiating between distinct phases, i.e. molecular variants, of highly homologous molecules.
8. The method of Embodiment 7, wherein the method is performed with a plurality of clonal nucleic acid populations each having a different molecule-specific barcodes attached thereto, and a separate sequence is assembled in (h) for each of the molecule-specific barcode.
9. A method for tagging single nucleic acid molecules for single-cell synthetic long-read (SLR) DNA sequencing or RNA sequencing, the method comprising:
   (a) encapsulating single cells into individual partitions and extracting its nucleic acid content inside each partition;
   (b) tagging the nucleic acid molecules inside each partition with partition-specific barcodes on one terminal end;
   (c) tagging the nucleic acid molecules with unique molecule-specific barcodes on the opposing terminal end, thereby obtaining a pool of uniquely barcoded nucleic acid molecules;
   (d) providing a plurality of clonal nucleic acid molecules each having the same partition-specific and molecule-specific barcodes at the terminal ends;
   (e) appending the terminal end containing the molecule-specific barcodes with an elongation sequence that is also internal to the long nucleic acid molecule;
   (f) for each nucleic acid molecule, denaturing and obtaining single-stranded nucleic acids with the elongation sequence on the 3′ terminal end for intramolecular priming;
   (g) for each nucleic acid molecule, annealing the 3′ terminal end with the elongation sequence at an internal position intramolecularly and extending the molecule;
   (h) for each nucleic acid molecule, sequencing the partition-specific barcode, the molecule-specific barcode, and the internal sequences downstream of the elongation sequence;
   (i) assembling the sequence of the nucleic acid molecule from the plurality of internal sequences of the nucleic acid molecule; and
   (j) differentiating between distinct phases, i.e. molecular variants, of highly homologous molecules.
10. The method of Embodiment 1, Embodiment 3, Embodiment 5, Embodiment 7, or Embodiment 9, wherein the tagging in (b) is performed by primer extension.
11. The method of Embodiment 1, Embodiment 3, Embodiment 5, Embodiment 7, or Embodiment 9, wherein the tagging in (b) is performed by reverse transcription.
12. The method of Embodiment 1, Embodiment 3, Embodiment 5, Embodiment 7, or Embodiment 9, wherein the tagging in (b) is performed by ligation.
13. The method of Embodiment 1, Embodiment 3, Embodiment 5, Embodiment 7, or Embodiment 9, wherein the nucleic acid molecules are fragmented prior to terminal barcode tagging in (b).
14. The method of Embodiment 1, Embodiment 3, Embodiment 5, Embodiment 7, or Embodiment 9, wherein the nucleic acid molecules are amplified and fragmented prior to terminal barcode tagging (b).
15. The method of Embodiment 3, Embodiment 5, or Embodiment 9, wherein the tagging in (c) is performed by primer extension.
16. The method of Embodiment 3, Embodiment 5, or Embodiment 9, wherein the tagging in (c) is performed by ligation.
17. The method of Embodiment 1 or Embodiment 7, wherein the providing plurality in (c) is performed by PCR.
18. The method of Embodiment 3, Embodiment 5, or Embodiment 9, wherein the tagging in (c) takes place inside the single-cell partition.
19. The method of Embodiment 3, Embodiment 5, or Embodiment 9, wherein the tagging in (c) takes place after the partitions are broken and all the barcode-tagged nucleic acid molecules are pooled.
20. The method of Embodiment 3, Embodiment 5, or Embodiment 9, wherein the providing plurality in (d) is performed by PCR.
21. The method of Embodiment 1 or Embodiment 7, wherein the terminal tags comprising partition-specific and unique molecule-specific barcodes are immobilized on microparticles, each microparticle comprising many copies of tags with identical partition-specific barcodes but different molecule-specific barcodes.
22. The method of Embodiment 21, further comprising the barcoded microparticles co-encapsulated with single cells in aqueous solution.
23. The method of Embodiment 21, further comprising that each partition comprises a single microparticle and a single cell.
24. The method of Embodiment 21, further comprising that the barcoded microparticles are in a suspension of cell lysis buffer, such that the lysis buffer is co-encapsulated in the aqueous solution alongside the microparticle and individual cells.
25. The method of Embodiment 1 or Embodiment 7, wherein the terminal tags comprising partition-specific and unique molecule-specific barcodes are formed into aqueous droplets, each droplet comprising many copies of tags with identical partition-specific barcodes but different molecule-specific barcodes, thereby producing barcoded droplets.
26. The method of Embodiment 25, wherein the barcoded droplets are fused with aqueous droplets with single-cell partitions.
27. The method of Embodiment 25, further comprising that the barcode tags are in a suspension of cell lysis buffer, such that the lysis buffer is co-encapsulated in the aqueous solution when the barcode tags droplets are fused with single-cell droplets.
28. The method of Embodiment 3, Embodiment 5, or Embodiment 9, wherein the terminal tags comprising partition-specific barcodes are immobilized on microparticles, each microparticle comprising many copies of tags with identical partition-specific barcodes.
29. The method of Embodiment 28, further comprising the barcoded microparticles co-encapsulated with single cells in aqueous solution.
30. The method of Embodiment 28, further comprising that each partition comprises a single microparticle and a single cell.
31. The method of Embodiment 28, further comprising that the barcoded microparticles are in a suspension of cell lysis buffer, such that the lysis buffer is co-encapsulated in the aqueous solution alongside the microparticle and individual cells.
32. The method of Embodiment 3, Embodiment 5, or Embodiment 9, wherein the terminal tags comprising partition-specific barcodes are formed into aqueous droplets, each droplet comprising many copies of tags with identical partition-specific barcodes but different molecule-specific barcodes, thereby producing barcoded droplets.
33. The method of Embodiment 32, further comprising that the barcoded droplets are fused with aqueous droplets with single-cell partitions.
34. The method of Embodiment 32, further comprising that the barcode tags are in a suspension of cell lysis buffer, such that the lysis buffer is co-encapsulated in the aqueous solution when the barcode tags droplets are fused with single-cell droplets.
35. A method of obtaining nucleic acid sequence information from a nucleic acid molecule by assembling a plurality of short nucleic acid sequences into a longer nucleic acid sequence, said method comprising:
   (a) attaching a terminal tag comprising a sequencing adapter sequence, a universal PCR sequence, a partition-specific barcode, and a molecule-specific barcode, with or without a target molecule sequence to one end of a plurality of nucleic acid molecules to form a pool of barcode-tagged molecules;
   (b) attaching a second terminal tag on the opposing end of the barcode tag, comprising a universal PCR sequence, with or without a target molecule sequence;
   (c) amplifying the barcode-tagged molecules to obtain a library of barcode-tagged molecules with many copies of identical molecules;
   (d) fragmenting the barcode-tagged molecules, thereby generating barcode-tagged fragments comprising of the barcode sequence on one end and an unknown sequence from an internal region on the other end;
   (e) circularizing the barcode-tagged fragments comprising of the barcode sequence on one end and an unknown sequence from an internal region on the other end via intramolecular ligation, thereby bringing the barcode sequence into proximity with the unknown sequence from an internal region;
   (f) fragmenting the circularized, barcode-tagged fragments into linear, barcode-tagged molecule, with the barcode sequence at the internal region of the linear molecule;
   (g) attaching a second sequencing adapter to each end of the linear barcoded-fragment to form double adapter-ligated barcode-tagged nucleic acid fragments;
   (h) amplifying all or part of the double adapter-ligated barcode-tagged nucleic acid fragments;
   (i) sequencing the double adapter-ligated barcode-tagged nucleic acid fragments;
   (j) clustering the sequenced nuclear acid fragments into groups using the molecule-specific barcodes; and
   (k) assembling each group of reads with the same molecule-specific barcodes into long nucleic acid sequence.
36. The method of Embodiment 35, wherein the target molecule sequence on the barcode tag comprises poly-thymine repeats and the target molecule sequence on the opposing tag comprises poly-guanine repeats.
37. The method of Embodiment 35, wherein the target molecule sequence on the barcode tag comprises gene-specific sequence bracketing one end of the region of interest and the target molecule sequence on the opposing tag comprises poly-guanine repeats.
38. The method of Embodiment 35 wherein the target molecule sequence on the barcode tag comprises gene-specific sequence bracketing one end of the region of interest and the target molecule sequence on the opposing tag comprises a second gene-specific sequence bracketing the other end of the region of interest.
39. The method of Embodiment 35, wherein the target molecule sequence on the barcode tag comprises poly-guanine repeats and the target molecule sequence on the opposing tag comprises poly-thymine repeats.
40. The method of Embodiment 35, wherein the target molecule sequence on the barcode tag comprises poly-thymine repeats.
41. The method of Embodiment 35, wherein the target molecule sequence on the barcode tag comprises gene-specific sequence.
42. The method of Embodiment 35, wherein the target molecule sequence on the barcode tag comprises a random sequence of a length of at least 6 bases.
43. The method of Embodiment 35, wherein the target molecule sequence on the barcode tag comprises a random sequence of a length of at least 8 bases.
44. The method of Embodiment 35, wherein the target molecule sequence on the barcode tag comprises a random sequence of a length of at least 10 bases.
45. The method of Embodiment 35, wherein the target molecule sequence on the barcode tag comprises a random sequence of a length of at least 12 bases.
46. The method of Embodiment 35, wherein the target molecule sequence on the barcode tag comprises a random sequence of a length of at least 16 bases.
47. The method of Embodiment 35, wherein the target molecule sequence on the barcode tag comprises a random sequence of a length of at least 20 bases.
48. A method of obtaining nucleic acid sequence information from a nucleic acid molecule by assembling a plurality of short nucleic acid sequences into a longer nucleic acid sequence, said method comprising:
   (a) attaching a terminal tag comprising a universal PCR sequence and a partition-specific barcode, with or without a target molecule sequence to one end of a plurality of nucleic acid molecules to form a pool of barcode-tagged molecules;
   (b) attaching a second terminal tag on the opposing end of the first barcode tag, comprising a sequencing adapter sequence, a universal PCR sequence, and a molecule-specific barcode, with or without a target molecule sequence;
   (c) amplifying the barcode-tagged molecules to obtain a library of barcode-tagged molecules with many copies of identical molecules;
   (d) fragmenting the barcode-tagged molecules, thereby generating barcode-tagged fragments comprising of the barcode sequence on one end and an unknown sequence from an internal region on the other end;
   (e) circularizing the barcode-tagged fragments comprising of the barcode sequence on one end and an unknown sequence from an internal region on the other end via intramolecular ligation, thereby bringing the barcode sequence into proximity with the unknown sequence from an internal region;
   (f) fragmenting the circularized, barcode-tagged fragments into linear, barcode-tagged molecule, with the barcode sequence at the internal region of the linear molecule;
   (g) attaching a second sequencing adapter to each end of the linear barcoded-fragment to form double adapter-ligated barcode-tagged nucleic acid fragments;
   (h) amplifying all or part of the double adapter-ligated barcode-tagged nucleic acid fragments;
   (i) sequencing the double adapter-ligated barcode-tagged nucleic acid fragments;
   (j) clustering the sequenced nuclear acid fragments into groups using the molecule-specific barcodes; and
   (k) assembling each group of reads with the same molecule-specific barcodes into long nucleic acid sequence.
49. The method of Embodiment 48, wherein the target molecule sequence on the partition-specific barcode tag comprises poly-thymine repeats and the target molecule sequence on the molecule-specific tag comprises poly-guanine repeats.
50. The method of Embodiment 48, wherein the target molecule sequence on the partition-specific barcode tag comprises gene-specific sequence bracketing one end of the region of interest and the target molecule sequence on the molecule-specific tag comprises poly-guanine repeats.
51. The method of Embodiment 48, wherein the target molecule sequence on the partition-specific barcode tag comprises gene-specific sequence bracketing one end of the region of interest and the target molecule sequence on the molecule-specific tag comprises a second gene-specific sequence bracketing the other end of the region of interest.
52. The method of Embodiment 48, wherein the target molecule sequence on the partition-specific barcode tag comprises poly-guanine repeats and the target molecule sequence on the molecule-specific barcode tag comprises poly-thymine repeats.
53. The method of Embodiment 48, wherein the target molecule sequence on the partition-specific barcode tag comprises a poly-thymine repeats.
54. The method of Embodiment 48, wherein the target molecule sequence on the partition-specific barcode tag comprises a gene-specific sequence.
55. The method of Embodiment 48, wherein the target molecule sequence on the partition-specific barcode tag comprises a random sequence of a length of at least 6 bases.
56. The method of Embodiment 48, wherein the target molecule sequence on the partition-specific barcode tag comprises a random sequence of a length of at least 8 bases.
57. The method of Embodiment 48, wherein the target molecule sequence on the partition-specific barcode tag comprises a random sequence of a length of at least 10 bases.
58. The method of Embodiment 48, wherein the target molecule sequence on the partition-specific barcode tag comprises a random sequence of a length of at least 12 bases.
59. The method of Embodiment 48, wherein the target molecule sequence on the partition-specific barcode tag comprises a random sequence of a length of at least 16 bases.
60. The method of Embodiment 48, wherein the target molecule sequence on the partition-specific barcode tag comprises a random sequence of a length of at least 20 bases.
61. The method of Embodiment 35 or Embodiment 48, wherein the attaching in (a) takes place inside partition-specific partitions.
62. The method of Embodiment 35 or Embodiment 48, wherein the attaching in (a) is performed by primer extension.
63. The method of Embodiment 35 or Embodiment 48, wherein the attaching in (a) is performed by reverse transcription.
64. The method of Embodiment 35 or Embodiment 48, wherein the attaching in (a) is performed by ligation.
65. The method of Embodiment 35 or Embodiment 48, wherein the attaching in (b) takes place inside the single-cell partitions.
66. The method of Embodiment 35 or Embodiment 48, wherein the attaching in (b) takes place after the partitions are broken and all the barcode-tagged nucleic acid molecules are pooled.
67. The method of Embodiment 35 or Embodiment 48, wherein the attaching in (b) is performed by primer extension.
68. The method of Embodiment 35 or Embodiment 48, wherein the attaching in (b) is performed by ligation.
69. The method of Embodiment 35 or Embodiment 48, wherein the nucleic acid molecules are fragmented prior to tagging with molecule-specific barcode in (b).
70. The method of Embodiment 35 or Embodiment 48, wherein the amplifying in (c) is performed by PCR.
71. The method of Embodiment 70, further comprising the use of an uracil-tolerance DNA polymerase and uracil-containing universal PCR primers.
72. The method of Embodiment 71, wherein the uracil-containing universal region is removed prior to circularization in (e).
73. The method of Embodiment 35 or Embodiment 48, wherein the circularizing in (e) is performed by ligation.
74. A method of obtaining nucleic acid sequence information from a nucleic acid molecule by assembling a plurality of short nucleic acid sequences into a longer nucleic acid sequence, said method comprising:
   (a) attaching a terminal tag comprising a sequencing adapter sequence, a universal PCR sequence, a partition-specific barcode, and a molecule-specific barcode, with or without a target molecule sequence to one end of a plurality of nucleic acid molecules to form a pool of barcode-tagged molecules;
   (b) attaching a second terminal tag on the opposing end of the barcode tag, comprising a universal PCR sequence, with or without a target molecule sequence;
   (c) amplifying the barcode-tagged molecules to obtain a library of barcode-tagged molecules with many copies of identical molecules;
   (d) appending the terminal end containing barcodes with an elongation sequence that is also internal to the long nucleic acid molecule;
   (e) denaturing or removing one of the two strands of the double-stranded barcoded-tagged molecule with elongation sequence, thereby generating barcode-tagged molecules comprising of the barcode sequence and an elongation sequence on the 3′ end;
   (f) annealing the 3′ terminal end with the elongation sequence at an internal position intramolecularly and extending the molecule, thereby bringing the barcode sequence into proximity with the internal region that is complementary to the elongation sequence;
   (g) attaching a second sequencing adapter to the intramolecularly elongated barcoded molecule to form double-adapter barcode-tagged nucleic acid fragments;
   (h) amplifying all or part of the double-adapter barcode-tagged nucleic acid fragments;
   (i) sequencing the double-adapter barcode-tagged nucleic acid fragments;
   (j) clustering the sequenced nuclear acid fragments into groups using the molecule-specific barcodes; and
   (k) assembling each group of reads with the same molecule-specific barcodes into long nucleic acid sequence.
75. A method of obtaining nucleic acid sequence information from a nucleic acid molecule by assembling a plurality of short nucleic acid sequences into a longer nucleic acid sequence, said method comprising:
   (a) attaching a terminal tag comprising a universal PCR sequence, and a partition-specific barcode, with or without a target molecule sequence to one end of a plurality of nucleic acid molecules to form a pool of barcode-tagged molecules;
   (b) attaching a second terminal tag on the opposing end of the partition-specific barcode tag, comprising a sequencing adapter sequence, a universal PCR sequence, and a molecule-specific barcode, with or without a target molecule sequence;
   (c) amplifying the barcode-tagged molecules to obtain a library of barcode-tagged molecules with many copies of identical molecules;
   (d) appending the terminal end containing barcodes with an elongation sequence that is also internal to the long nucleic acid molecule;
   (e) denaturing or removing one of the two strands of the double-stranded barcoded-tagged molecule with elongation sequence, thereby generating barcode-tagged molecules comprising of the barcode sequence and an elongation sequence on the 3′ end;
   (f) annealing the 3′ terminal end with the elongation sequence at an internal position intramolecularly and extending the molecule, thereby bringing the barcode sequence into proximity with the internal region that is complementary to the elongation sequence;
   (g) attaching a second sequencing adapter to the intramolecularly elongated barcoded molecule to form double-adapter barcode-tagged nucleic acid fragments;
   (h) amplifying all or part of the double-adapter barcode-tagged nucleic acid fragments;
   (i) sequencing the double-adapter barcode-tagged nucleic acid fragments;
   (j) clustering the sequenced nuclear acid fragments into groups using the molecule-specific barcodes; and
   (k) assembling each group of reads with the same molecule-specific barcodes into long nucleic acid sequence.
76. The method of Embodiment 74 or Embodiment 75, wherein the attaching in (a) takes place inside partition-specific partitions.
77. The method of Embodiment 74 or Embodiment 75, wherein the attaching in (a) is performed by primer extension.
78. The method of Embodiment 74 or Embodiment 75, wherein the attaching in (a) is performed by reverse transcription.
79. The method of Embodiment 74 or Embodiment 75, wherein the attaching in (a) is performed by ligation.
80. The method of Embodiment 74 or Embodiment 75, wherein the attaching in (b) takes place inside the single-cell partitions.
81. The method of Embodiment 74 or Embodiment 75, wherein the attaching in (b) takes place after the partitions are broken and all the barcode-tagged nucleic acid molecules are pooled.
82. The method of Embodiment 74 or Embodiment 75 wherein the attaching in (b) is performed by primer extension.
83. The method of Embodiment 74 or Embodiment 75, wherein the attaching in (b) is performed by ligation.
84. The method of Embodiment 74 or Embodiment 75, further comprising the nucleic acid molecules are fragmented prior to the attaching in (b).
85. The method of Embodiment 74 or Embodiment 75, wherein the amplifying in (c) is performed by PCR.
86. The method of Embodiment 74 or Embodiment 75, wherein the amplifying in (d) is performed by PCR.
87. The method of Embodiment 74 or Embodiment 75, wherein the amplifying in (d) is performed by ligation.
88. The method of Embodiment 74 or Embodiment 75, wherein different elongation sequences are appended to different copies of the nucleic acid molecules sharing the same molecule-specific barcode, thereby generating a pool of barcode-tagged nucleic acids with different elongation sequences complementary to different internal positions. Collectively, the different internal positions cover the length of the nucleic acid molecule or discontiguous regions of interest by design.
89. The method of Embodiment 74 or Embodiment 75, wherein the elongation sequence on the barcode tag comprises a random sequence of a length of at least 6 bases.
90. The method of Embodiment 74 or Embodiment 75, wherein the elongation sequence on the barcode tag comprises a random sequence of a length of at least 8 bases.
91. The method of Embodiment 74 or Embodiment 75, wherein the elongation sequence on the barcode tag comprises a random sequence of a length of at least 10 bases.
92. The method of Embodiment 74 or Embodiment 75, wherein the elongation sequence on the barcode tag comprises a random sequence of a length of at least 12 bases.
93. The method of Embodiment 74 or Embodiment 75, wherein the elongation sequence on the barcode tag comprises a random sequence of a length of at least 16 bases.
94. The method of Embodiment 74 or Embodiment 75, wherein the elongation sequence on the barcode tag comprises a random sequence of a length of at least 20 bases.
95. The method of Embodiment 74 or Embodiment 75, wherein the generating ssDNA in (e) is performed by heat denaturation under dilute condition.
96. The method of Embodiment 74 or Embodiment 75, wherein the generating ssDNA in (e) is performed by alkaline denaturation under dilute condition.
97. The method of Embodiment 74 or Embodiment 75, wherein the generating ssDNA in (e) is performed by 5′ phosphorylation of the strand to be removed and enzymatic digestion by lambda exonuclease.
98. The method of Embodiment 74 or Embodiment 75, wherein the generating ssDNA in (e) is performed by appending the strand to be removed with 5′ biotinylation, immobilizing the strand on streptavidin-coated solid-surface, and releasing the strand for elongation through washing and/or denaturation.
99. The method of Embodiment 74 or Embodiment 75, wherein the extension in (f) is performed isothermally.
100. The method of Embodiment 74 or Embodiment 75, wherein the extension in (f) is performed by primer annealing at one temperature and extension at a different temperature.
101. The method of Embodiment 74 or Embodiment 75, wherein the attaching in (g) is performed by PCR by using primers that contain the second sequencing adapter and a gene-specific sequence downstream of the elongation sequence.
102. The method of Embodiment 74 or Embodiment 75, further comprising fragmenting the barcode-tagged and elongated nucleic acid molecules prior to attaching in (g).
103. The method of any one of the Embodiments 1 to 102, wherein the nucleic acid sequence is obtained for a longer nucleic acid sequence comprising a length of at least about 500 bases.
104. The method of any one of the Embodiments 1 to 103, wherein the nucleic acid sequence is obtained for a longer nucleic acid sequence comprising a length of at least about 1000 bases.
105. The method of any one of the Embodiments 1 to 104, wherein the nucleic acid sequence is obtained for a longer nucleic acid sequence comprising a length of at least 1000 or more bases.
106. The method of any one of the Embodiments 1 to 105, wherein the nucleic acid sequence is obtained for a longer nucleic acid sequence comprising a length of at least 1 kilobases to about 20 kilobases.

1-86. (canceled)

87. A method comprising:
(a) providing a plurality of nucleic acid molecules from a single cell inside a partition;
(b) appending an adapter to an end of said plurality of nucleic acid molecules inside said partition, wherein said adapter comprises a partition-specific barcode and a molecule-specific barcode, thereby generating a plurality of barcoded nucleic acid molecules, wherein said partition-specific barcode is common to each of said plurality of barcoded nucleic acid molecules inside said partition;
(c) amplifying said plurality of barcoded nucleic acid molecules, thereby generating a plurality of amplified barcoded nucleic acid molecules;
(d) fragmenting said plurality of amplified barcoded nucleic acid molecules to generate a plurality of nucleic acid fragments, wherein a nucleic acid fragment from at least a portion of said plurality of nucleic acid fragments comprises a first end without said adapter and a second end comprising said adapter; and
(e) circularizing said plurality of nucleic acid fragments by ligating said first end to said second end of said nucleic acid fragment from said plurality of nucleic acid fragments, thereby generating a plurality of circularized nucleic acid molecules comprising said adapter.

88. The method of claim 87, further comprising sequencing said plurality of circularized nucleic acid molecules to generate sequencing reads.

89. The method of claim 88, further comprising clustering said sequencing reads using said molecule-specific barcodes to generate long read sequencing information for said plurality of nucleic acid molecules from said single cell.

90. The method of claim 87, further comprising encapsulating said single cell inside said partition prior to (a).

91. The method of claim 90, wherein said partition is an aqueous droplet.

92. The method of claim 90, wherein said partition comprises the single cell and a single microparticle.

93. The method of claim 87, further comprising extracting said plurality of nucleic acid molecules inside said partition.

94. The method of claim 87, wherein said plurality of nucleic acid molecules comprises deoxyribonucleic acid (DNA).

95. The method of claim 92, wherein said DNA comprises complementary deoxyribonucleic acid (cDNA).

96. The method of claim 93, wherein said cDNA is derived ribonucleic acid (RNA) from said single cell.

97. The method of claim 94, further comprising, prior to (b), subjecting said RNA to reverse transcription to yield said cDNA.

98. The method of claim 87, wherein in (b) said adapter is appended to said end of said plurality of nucleic acid molecules via ligation.

99. The method of claim 87, wherein said appending in (b) is performed inside said partition.

100. The method of claim 87, wherein said adapter is appended to a 5′ end and a 3′ end of said plurality of nucleic acid molecules.

101. The method of claim 87, wherein said fragmenting comprises randomly fragmenting said amplified barcoded nucleic acid molecules.

102. The method of claim 88, further comprising phasing said sequencing reads to determine a molecular origin of two or more alleles in said plurality of nucleic acid molecules.

103. The method of claim 87, wherein at least a portion of said plurality of barcoded nucleic acid molecules comprises a unique molecule-specific barcode.

104. The method of claim 101, wherein a long read sequence is generated for said unique molecule-specific barcodes.

105. The method of claim 87, further comprising performing (a)-(e) in a plurality of partitions, wherein each partition comprises a plurality of nucleic acid molecules from a single cell.

106. The method of claim 87, further comprising sequencing said plurality of barcoded nucleic acid molecules to generate sequence reads and differentiating between sequence reads from different partitions based on said partition-specific barcode.


SEQUENCE LISTING (42 sequences)

SEQ ID NO: 1 - DNA, 12 bp, Artificial Sequence
  Description of Artificial Sequence Synthetic oligonucleotide
gcttccttctga

SEQ ID NO: 2 - DNA, 12 bp, Artificial Sequence
  Description of Artificial Sequence Synthetic oligonucleotide
gtcagaagcact

SEQ ID NO: 3 - protein, 6 aa, Artificial Sequence
  Description of Artificial Sequence Synthetic 6xHis tag
HHHHHH

SEQ ID NO: 4 - DNA, 12 bp, Artificial Sequence
  Description of Artificial Sequence Synthetic oligonucleotide
tcaatacagtta

SEQ ID NO: 5 - DNA, 12 bp, Artificial Sequence
  Description of Artificial Sequence Synthetic oligonucleotide
aacagtacctag

SEQ ID NO: 6 - DNA, 12 bp, Artificial Sequence
  Description of Artificial Sequence Synthetic oligonucleotide
tacctaacccgc

SEQ ID NO: 7 - DNA, 12 bp, Artificial Sequence
  Description of Artificial Sequence Synthetic oligonucleotide
cgaacaatcgac

SEQ ID NO: 8 - DNA, 12 bp, Artificial Sequence
  Description of Artificial Sequence Synthetic oligonucleotide
atagtagcgctt

SEQ ID NO: 9 - DNA, 12 bp, Artificial Sequence
  Description of Artificial Sequence Synthetic oligonucleotide
gatcagcatatc

SEQ ID NO: 10 - DNA, 12 bp, Artificial Sequence
  Description of Artificial Sequence Synthetic oligonucleotide
aaagcggacgaa

SEQ ID NO: 11 - DNA, 12 bp, Artificial Sequence
  Description of Artificial Sequence Synthetic oligonucleotide
cgcaccgacccc

SEQ ID NO: 12 - DNA, 12 bp, Artificial Sequence
  Description of Artificial Sequence Synthetic oligonucleotide
gcttccttctga

SEQ ID NO: 13 - DNA, 12 bp, Artificial Sequence
  Description of Artificial Sequence Synthetic oligonucleotide
tgactcctaaac

SEQ ID NO: 14 - DNA, 12 bp, Artificial Sequence
  Description of Artificial Sequence Synthetic oligonucleotide
acattactggac

SEQ ID NO: 15 - DNA, 12 bp, Artificial Sequence
  Description of Artificial Sequence Synthetic oligonucleotide
aagagatcgtaa

SEQ ID NO: 16 - DNA, 12 bp, Artificial Sequence
  Description of Artificial Sequence Synthetic oligonucleotide
gtcagaagcact

SEQ ID NO: 17 - DNA, 16 bp, Artificial Sequence
  Description of Artificial Sequence Synthetic oligonucleotide
catatggcagtcgatt

SEQ ID NO: 18 - DNA, 16 bp, Artificial Sequence
  Description of Artificial Sequence Synthetic oligonucleotide
ggtgcgtagtagccga

SEQ ID NO: 19 - DNA, 16 bp, Artificial Sequence
  Description of Artificial Sequence Synthetic oligonucleotide
ccttcgacaacacccg

SEQ ID NO: 20 - DNA, 16 bp, Artificial Sequence
  Description of Artificial Sequence Synthetic oligonucleotide
caccaggtcggcgcta

SEQ ID NO: 21 - DNA, 16 bp, Artificial Sequence
  Description of Artificial Sequence Synthetic oligonucleotide
acatacgagaggtacc

SEQ ID NO: 22 - DNA, 16 bp, Artificial Sequence
  Description of Artificial Sequence Synthetic oligonucleotide
acatcagcacggccat

SEQ ID NO: 23 - DNA, 16 bp, Artificial Sequence
  Description of Artificial Sequence Synthetic oligonucleotide
agtgtcatctaacttc

SEQ ID NO: 24 - DNA, 16 bp, Artificial Sequence
  Description of Artificial Sequence Synthetic oligonucleotide
cagctaaagacaagcc

SEQ ID NO: 25 - DNA, 16 bp, Artificial Sequence
  Description of Artificial Sequence Synthetic oligonucleotide
gtgcatagtcagataa

SEQ ID NO: 26 - DNA, 16 bp, Artificial Sequence
  Description of Artificial Sequence Synthetic oligonucleotide
tagttgggttaccagt

SEQ ID NO: 27 - DNA, 16 bp, Artificial Sequence
  Description of Artificial Sequence Synthetic oligonucleotide
gacgttacatcggtta

SEQ ID NO: 28 - DNA, 16 bp, Artificial Sequence
  Description of Artificial Sequence Synthetic oligonucleotide
cgcttcacacaacgtt

SEQ ID NO: 29 - DNA, 16 bp, Artificial Sequence
  Description of Artificial Sequence Synthetic oligonucleotide
ttcggtcgtctccatc

SEQ ID NO: 30 - DNA, 1274 bp, Homo sapiens
ccccgcctgcggcccagctccttcccgcggctctgcgatgcggcccgcagggtgacccgggcgggagtccggggacccgcgatcagccccggaggacggggtggggtcgccccaaacaggagcgccgggaccgctgggaccccgcactcggcgtccgccgccgccgggtagccgggcagtggaggtcccggatgaggcgacaatttttccggccccccctcccagtcccgccccacttccggggccgccactttcactttctcttccgccgaagccgctccccttgcgaagaactggggcctcccgggaggagagagggctttgccttgaaacccgggacgccaggggcgctcccgcaagtgggggtcctccgggacttggaacgccccggctgggtggtgtccgggcgtcctttccccgcttcttcccacctcggctggtcccgtttcctcctgcgcccagtgcggacctgtctcggcgcccgctgccctctcaccgccccacgcaggatcccggcctggtcaccgggcagtgtgatgcttcccgactgccgcggggacagcgaggcacacacagggcttgggccgcgccggaggccacacggcctggctgagttgctcctggtctcccgcctctcccaggcgacccggaggtagcatttcccaggaggcacggtcccccccagggggatgggcacagccacgccagatggacgagaagaccaagaaagcagaggaaatggccctgagcctcacccgagcagtggcgggcggggatgaacaggtggcaatgaagtgtgccatctggctggcagagcaacgggtgcccctgagtgtgcaactgaagcctgaggtctccccaacgcaggacatcagattcctcatggtgcaaaatggccattccagctccatccagccatcacatcacaggaggaagggaagaaagacacccctccacactcttctaaagagcatagctcaaaaattgtacacacttcttccgttaattcctgtggaccagaactgattcccacagctacagttcagcttgaggggagactgtatagccaagatattcagctagaattcaggggttcgcttggtaagggaagggaagagaatggatactgtcggtctgtgctccaggagacttaaactcaatgctgaaacactttgcacaatgcctggcgtgttatgcactcaataataaacattagtgtctatcgttaaaaaaaaaaaaaaaaaaaaaaaaaaaaaaaaaaaaaaaaaaaaaaa

SEQ ID NO: 31 - DNA, 2116 bp, Homo sapiens
actcccgggccgccgggggcactagggggggtggggtttccttccgcatctccacggttccaactccaacctagactcaaactggacgccggccggagactccgctccggcagcaaaccccacgtggtgcacctctgagcctccgcccctctcccgagggaaccgcaactctacttctcgcgagaattgcttctatggctccatcctgctttccggctgtcgccctcatgcgataggctctcagcgttacttgactcttctcgcgataattttttttaaaaatctcccaaggaaagttgaaggaagagtacaaaattttcatctcgcgagacttgtgagcggccatcttggtcctgccctgacagattctcctatcggggtcacagggacgctaagattgctacctggactttcgttgaccatgctgtcccgggtggtactttccgccgccgccacagcggccccctctctgaagaatgcagccttcctaggtccaggggtattgcaggcaacaaggacctttcatacagggcagccacaccttgtccctgtaccacctcttcctgaatacggaggaaaagttcgttatggactgatccctgaggaattcttccagtttctttatcctaaaactggtgtaacaggaccctatgtactcggaactgggcttatcttgtacgctttatccaaagaaatatatgtgattagcgcagagaccttcactgccctatcagtactaggtgtaatggtctatggaattaaaaaatatggtccctttgttgcagactttgctgataaactcaatgagcaaaaacttgcccaactagaagaggcgaagcaggcttccatccaacacatccagaatgcaattgatacggagaagtcacaacaggcactggttcagaagcgccattacctttttgatgtgcaaaggaataacattgctatggctttggaagttacttaccgggaacgactgtatagagtatataaggaagtaaagaatcgcctggactatcatatatctgtgcagaacatgatgcgtcgaaaggaacaagaacacatgataaattgggtggagaagcacgtggtgcaaagcatctccacacagcaggaaaaggagacaattgccaagtgcattgcggacctaaagctgctggcaaagaaggctcaagcacagccagttatgtaaatgtatctatcccaattgagacagctagaaacagttgactgactaaatggaaactagtctatttgacaaagtctttctgtgttggtgtctactgaagttatagtttacccttcctaaaaatgaaaagtttgtttcatatagtgagagaacgaaatctctatcggccagtcagatgtttctcatccttcttgctctgcctttgagttgttccgtgatcacttctgaataagcagtttgcctttataaaaacttgctgcctgactaaagattaacaggttatagtttaaatttgtaattaattctaccatcttgcaataaagtgacaattgaatgaaacagggtttttcaagttgtataattctctgaaatactcagcttttgtcatatgggtaaaaattaaagatgtcattgaactactgtcttgtttatgagaccattcagtggtgaactgtttctggctgataggttatgagatatgtaaagctttctagtactcttaaaataactaaatggagtattatatatcaattcatatcattgactttattattttagtagtatgcctatagaaaatattatggactcagagtgtcataaaatcactcttaagaatccatgcagcaggccaggcacagtggctcacacctgtaatgcctgcactttggaaggccgagacaggcggatcacttgaggtcaggagtttgaaaccagccaggccaacacagtgaaaccctgtctctactaaaaatacaaaaggttagccgggcatggtggcaggcgcctgtaatcccagctactcaggaggctgaggcaggagaattgcttgaacgcaggaggcaaaggttgcagtgagctgagatcacgccactgcactccagcctgggcaacagacctcgactccatctagaaaaaaaaaaaaaaaaa

SEQ ID NO: 32 - DNA, 1125 bp, Homo sapiens
ctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagtttatggcagagctttacaaataaaaaaaaaactggcttttctgacaaaaaaaaaaaaaaaa

SEQ ID NO: 33 - DNA, 852 bp, Homo sapiens
cgtttttcctctcttcagcgtggggcgcccacaatttgcgcgctctctttctgctgctccccagctctcggatacagccgacaccatgggtttcggagacctgaaaagccctgccggcctccaggtgctcaacgattacctggcggacaagagctacatcgaggggtatgtgccatcacaagcagatgtggcagtatttgaagccgtgtccagcccaccgcctgccgacttgtgtcatgccctacgttggtataatcacatcaagtcttacgaaaaggaaaaggccagcctgccaggagtgaagaaagctttgggcaaatatggtcctgccgatgtggaagacactacaggaagtggagctacagatagtaaagatgatgatgacattgacctctttggatctgatgatgaggaggaaagtgaagaagcaaagaggctaagggaagaacgtcttgcacaatatgaatcaaagaaagccaaaaaacctgcacttgttgccaagtcttccatcttactagatgtgaaaccttgggatgatgagacagatatggcgaaattagaggagtgcgtcagaagcattcaagcagacggcttagtctggggctcatctaaactagttccagtgggatacggaattaagaaacttcaaatacagtgtgtagttgaagatgataaagttggaacagatatgctggaggagcagatcactgcttttgaggactatgtgcagtcgatggatgtggctgctttcaacaagatctaaaatccatcctggatcatggcatttaaataaaagattgaaagattaaaaaaaaaaaaaaaaaaaaaaaaaaaaagaaaaaaaaaaaaaaa

SEQ ID NO: 34 - DNA, 1325 bp, Homo sapiens
ggggctctaccggcgggatttgatggcgtgatgtctcacagaaagttctccgctcccagacatgggtccctcggcttcctgcctcggaagcgcagcagcaggcatcgtgggaaggtgaagagcttccctaaggatgacccgtccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaatgccaggaacagattttgcagttggtggggtctcaataaaagttattttccactgaaaaaaaaaaaaaaaaaaaaaaaaaaaa

SEQ ID NO: 35 - DNA, 1186 bp, Homo sapiens
cacttctgccgcccctgtttcaagggataagaaaccctgcgacaaaacctcctccttttccaagcggctgccgaagatggcggaggtgcaggtcctggtgcttgatggtcgaggccatctcctgggccgcctggcggccatcgtggctaaacaggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 36 - DNA, 1458 bp, Homo sapiens
aagcacttccttttcctgtggcagcagccgggctgagaggagcgtggctgtctcctctctccgccatggcgtgtgctcgcccactgatatcggtgtactccgaaaagggggagtcatctggcaaaaatgtcactttgcctgctgtattcaaggctcctattcgaccagatattgtgaactttgttcacaccaacttgcgcaaaaacaacagacagccctatgctgtcagtgaattagcaggtcatcagactagtgctgagtcttggggtactggcagagctgtggctcgaattcccagagttcgaggtggtgggactcaccgctctggccagggtgcttttggaaacatgtgtcgtggaggccgaatgtttgcaccaaccaaaacctggcgccgttggcatcgtagagtgaacacaacccaaaaacgatacgccatctgttctgccctggctgcctcagccctaccagcactggtcatgtctaaaggtcatcgtattgaggaagttcctgaacttcctttggtagttgaagataaagttgaaggctacaagaagaccaaggaagctgttttgctccttaagaaacttaaagcctggaatgatatcaaaaaggtctatgcctctcagcgaatgagagctggcaaaggcaaaatgagaaaccgtcgccgtatccagcgcaggggcccgtgcatcatctataatgaggataatggtatcatcaaggccttcagaaacatccctggaattactctgcttaatgtaagcaagctgaacattttgaagcttgctcctggtgggcatgtgggacgtttctgcatttggactgaaagtgctttccggaagttagatgaattgtacggcacttggcgtaaagccgcttccctcaagagtaactacaatcttcccatgcacaagatgattaatacagatcttagcagaatcttgaaaagcccagagatccaaagagcccttcgagcaccacgcaagaagatccatcgcagagtcctaaagaagaacccactgaaaaacttgagaatcatgttgaagctaaacccatatgcaaagaccatgcgccggaacaccattcttcgccaggccaggaatcacaagctccgggtggataaggcagctgctgcagcagcggcactacaagccaaatcagatgagaaggcggcggttgcaggcaagaagcctgtggtaggtaagaaaggaaagaaggctgctgttggtgttaagaagcagaagaagcctctggtgggaaaaaaggcagcagctaccaagaaaccagcccctgaaaagaagcctgcagagaagaaacctactacagaggagaagaagcctgctgcataaactcttaaatttgattattccataaaggtcaaatcattttggacagcttcttttgaataaagacctgattatacaggcagtgagaaacatgaaaaaaaaaaaaaaaaaa

SEQ ID NO: 37 - DNA, 4565 bp, Homo sapiens
gggcgggacttccgccgtcctcctggtggtggtcgttttggttctgtgtggtgtttcaccaacttcggcctatggctctgtctgacgtcaccgaagtgacggaacggaaaagcgcgagaagcggctcggttcccaccacggagaggcgggagtgagtcaactgacaagcgctggggacagtggcgtccttgtcttgcctttgtcgctcccgccccgctcttccctggctgggctggcggaggccttgctgatgaacctgactgagggtcccctggcgatggcagaaatggaccctacacagggccgtgtggtctttgaggacgtggccatatatttctcccaggaggagtgggggcaccttgatgaggctcagagattgctgtaccgtgatgtgatgctggagaatttggcccttttgtcctcactaggttcttggcatggagctgaggatgaggaggcaccttcacagcaaggtttttctgtaggagtgtcagaggttacagcttcaaagccctgtctgtccagccagaaggtccaccctagtgagacatgtggcccacccttgaaagacattctgtgcctggttgagcacaatggaattcatcctgagcaacacatatatatttgtgaggcagagctttttcagcacccaaagcagcaaattggagaaaatctttccagaggggatgattggataccttcatttgggaagaaccacagagttcacatggcagaggagatcttcacatgcatggagggctggaaggacttaccagccacctcatgccttctccagcaccagggccctcaaagcgagtggaagccatacagggacacagaggacagagaagcctttcagactggacaaaatgattacaaatgtagtgaatgtgggaaaaccttcacctgcagctattcatttgttgagcaccagaaaatccacacaggagaaaggtcttatgaatgtaacaaatgtgggaaattctttaagtacagtgccaatttcatgaaacatcagacagttcacactagtgaaaggacttatgagtgcagagaatgtggaaaatcctttatgtacaactaccgactcatgagacataagcgagttcacactggagaaaggccttatgagtgcaacacatgtgggaaattctttcggtacagctccacatttgttagacatcagagagttcacaccggagaaaggccgtatgagtgcagggaatgtgggaaattctttatggacagctccacactcattaaacatcagagagttcacaccggagaaagaccttataagtgcaatgattgtgggaaattttttaggtatatctccacactcattagacatcagagaattcacactggagaaaggccttatgagtgcagtgtatgtggggaattgtttaggtacaactccagccttgttaaacattggagaaatcacactggagaaaggccttataaatgcagtgaatgtgggaaatcatttaggtaccactgcaggctcattagacaccagagagtccacacgggagaaaggccttatgagtgcagcgaatgcgggaaattctttcgttacaactccaacctcattaaacattggagaaatcacactggagaaaggccttacgagtgcagagagtgtgggaaagcctttagccacaagcatatacttgttgagcaccagaaaatccacagtggagaaagaccttatgagtgcagcgaatgccagaaggcctttattagaaagtctcacctggttcatcaccagaaaatccacagtgaagagaggcttgtgtgctccatgaatgtggggaattctttagctaaaactccaacctcattaaacatcagagatttcacaatggagaaagtttaccattgactattgtaattgggtagtaatgttatataaattccacatttttatgcaactaatctccagaacatttttcctcttaccaagaagtaaaatgctgtacccattaacaacaactcattccccttccctacttccccagaaatgtctcaactatatttctatactctatggtacttatatgaggtaccaatagatatctatgaatttgatatatatttgtacctcatataagtggattctacagtatttatcttttgagactggcttatttcacttaggataaggtcttcacggttcacccatgttgtataatgtgtcagaatatccttcctttttaggtgaaataatattctatggtatttatataccacatttatttatccattcatctgttagtggatacttgggctacttccaccttttgcctattgaaataatgctgctatgaagatgagtgtacaagtgtctattcaagattctactttcaattcttatagggtatatactcagaaatggtggtgctggatcatataggatttctattttttttttttgtttgtttttgagacagagtcttgctctgtcacccaggctggagtgcagtgctgtgatcttggctcactgcaagctccgcctcccaggttcatgccattctcctgcctcaccctcccgagtagctgggactacaggtgcctgccaccacgcctggctaatttttttgtatttttagtagagacggggtttcaccgtgttagccaggatggtcctgatctcctgaccttgtgatctgcctgccttggcctctcaaagtgctgggattacgggcgtgagccaccgcgcctggccaggatttctatttttaatatttttgggaaaatttttccatagtacctgtgccattttacattcccaccagcagtgcacaaggattgcaatctatatacatcctcaccaacattgttcattttctatttctgtttttggggttttttgtagtgccttttgttttggatagcagctatcttgttggatgtgaggtggaatctatagtgtctttcatttttattttgtgaatgattgatgatgttgaggatcttttcatgtgcttgttaggcatttgtgtatctggaaaaatattcaagtcttttttttccatttttaatgggactatttgctttttgttgttgagttgtagttctttatacattctggatattaactccttaccaaatatatgctttttacatattacctcccagtccataggttgctttttcgctctgttgattgtgtcctttgatgaaattttaagttttgatgtactgttgactctttctgtctgtgggttctgtattcatggatcgaagcaaccatggatcaaaagtatttggagcatccatggattgcagtgatcattaatcaaaaatatttggaaaacaaaaagggtagttgcatctgtactaaacatgaacagacattttttcttgtcattattccctaaactatatagtataataaatatttacatagcatttacattgtattagaagttataaataacctaatgataatctatataggaagatgtgtgtaggttatattcaaacactatgcctttttatgtgagggacctcttgagcatcagatgattttggtatccacaaggggtcctggaatcagtcccccacagacaccaagggatgactgtagtgcattttatctatttttacttctgttacctgggcttttgatgttatatattaaaaaaaattagtatcaaatccaatgccaagcattttccctatgctttattctaagaattttatatttgaaggtcttacatttaggtcttttttttttttttcttttggaggcagagtcttgctctgtcacccagcctggagtgcagtagtggaatctcagctcactacaacctccgcctcctgggttcgagccatcaccccacctcagcctcccaagtagcttggattacaagtgtacaccaccacacctggctaatttttgtatttttagtagagatggggttttgccatgttggccaggctggtcttaaacttctggccttaagtgatccccctgcctcggcctcccaaattgctgagattacaggcaggagttgtaatgcactgtgcctggctacatttagatctttaatctacttggggttcatttttgcatatggtttaaggcaaaagtccactttatgtggctatccagttttccaagcaccattttttgaaaagagcatctttcctctgttgagtagtcttggcacacttgtcaaaatcatttgtccatatatgccatggtttatatgtggattctctattttattggtcatatgtctgtctttatgtcagtaccacacattttaggtgtgtgtgtgtgagactcagtgttgaggacaaggctagtgggctttcacactccagactgctgtattccagcccaaattactcaaattagccaatccatggggaacatggaaaacgtagctaatgcaatccgcttgccttacctaagttgtcccctgcagcctcaggttgctgttactgtgtttcagatgcaaccctctgtgggaccctacccaagttctctcattcttagctataggtaataaattgttctgattttgtgtatccaaaaaaaaaaaaaaaaa

SEQ ID NO: 38 - DNA, 1681 bp, Homo sapiens
cttgggagctggagcaggtggtggaggcctgaaatgactacttgagcttactcacatagcatattggtatatcaaaatgaaatgcaaggaaccaaaaataacataattgaaggcagtaaaagtgaaattaaataggaagatcatcagtcaaggaagacccactggagaggacagaaaatgaagcagtgttttatcatgtgtatttcagcaggtcttcttgaaatttaactaaaaatatgactgctctctcttcagagaactgctcttttcagtaccagttacgtcaaacaaaccagcccctagatgttaactatctgctattcttgatcatacttgggaaaatattattaaatatccttacactaggaatgagaagaaaaaacacctgtcaaaattttatggaatatttttgcatttcactagcattcgttgatcttttacttttggtaaacatttccattatattgtatttcagggattttgtacttttaagcattaggttcactaaataccacatctgcctatttactcaaattatttcctttacttatggctttttgcattatccagttttcctgacagcttgtatagattattgcctgaatttctctaaaacaaccaagctttcatttaagtgtcaaaaattattttatttctttacagtaattttaatttggatttcagtccttgcttatgttttgggagacccagccatctaccaaagcctgaaggcacagaatgcttattctcgtcactgtcctttctatgtcagcattcagagttactggctgtcatttttcatggtgatgattttatttgtagctttcataacctgttgggaagaagttactactttggtacaggctatcaggataacttcctatatgaatgaaactatcttatattttcctttttcatcccactccagttatactgtgagatctaaaaaaatattcttatccaagctcattgtctgttttctcagtacctggttaccatttgtactacttcaggtaatcattgttttacttaaagttcagattccagcatatattgagatgaatattccctggttatactttgtcaatagttttctcattgctacagtgtattggtttaattgtcacaagcttaatttaaaagacattggattacctttggatccatttgtcaactggaagtgctgcttcattccacttacaattcctaatcttgagcaaattgaaaagcctatatcaataatgatttgttaatattattaattaaaagttacagctgtcataagatcataattttatgaacagaaagaactcaggacatattaaaaaataaactgaactaaaacaacttttgccccctgactgatagcatttcagaatgtgtcttttgaagggctatgataccagttattaaatagtgttttattttaaaaacaaaataattccaagaagtttttatagttattcagggacactatattacaaatattactttgttattaacacaaaaagtgataagagttaacatttggctatactgatgtttgtgttactcaaaaaaactactggatgcaaactgttatgtaaatctgagatttcactgacaactttaagatatcaacctaaacatttttattaaatgttcaaatgaaagcaagaaagtaaaaattgttcttaaaatg

SEQ ID NO: 39 - DNA, 1028 bp, Homo sapiens
ccttttcccaccccctagcgccgctgggcctgcaggtctctgtcgagcagcggacgccggtctctgttccgcaggatggggtttgttaaagttgttaagaataaggcctactttaagagataccaagtgaaatttagaagacgacgagagggtaaaactgattattatgctcggaaacgcttggtgatacaagataaaaataaatacaacacacccaaatacaggatgatagttcgtgtgacaaacagagatatcatttgtcagattgcttatgcccgtatagagggggatatgatagtctgcgcagcgtatgcacacgaactgccaaaatatggtgtgaaggttggcctgacaaattatgctgcagcatattgtactggcctgctgctggcccgcaggcttctcaataggtttggcatggacaagatctatgaaggccaagtggaggtgactggtgatgaatacaatgtggaaagcattgatggtcagccaggtgccttcacctgctatttggatgcaggccttgccagaactaccactggcaataaagtttttggtgccctgaagggagctgtggatggaggcttgtctatccctcacagtaccaaacgattccctggttatgattctgaaagcaaggaatttaatgcagaagtacatcggaagcacatcatgggccagaatgttgcagattacatgcgctacttaatggaagaagatgaagatgcttacaagaaacagttctctcaatacataaagaacagcgtaactccagacatgatggaggagatgtataagaaagctcatgctgctatacgagagaatccagtctatgaaaagaagcccaagaaagaagttaaaaagaagaggtggaaccgtcccaaaatgtcccttgctcagaagaaggatcgggtagctcaaaagaaggcaagcttcctcagagctcaggagcgggctgctgagagctaaacccagcaattttctatgattttttcagatatagataataaacttatgaacagcaacta

SEQ ID NO: 40 - DNA, 1527 bp, Homo sapiens
gcggaagtgacgcgaggcgtagcggaagttactgcagccgcggtgttgtgctgtggggaagggagaaggatttgtaaaccccggagcgaggttctgcttacccgaggccgctgctgtgcggagacccccgggtgaagccaccgtcatcatgtctgaccaggaggcaaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggtcattcaacagtttagatattctttttattttttttcttttccctcaatccttttttatttttaaaaatagttcttttgtaatgtggtgttcaaaacggaattgaaaactggcaccccatctctttgaaacatctggtaatttgaattctagtgctcattattcattattgtttgttttcattgtgctgatttttggtgatcaagcctcagtccccttcatattaccctctcctttttaaaaattacgtgtgcacagagaggtcacctttttcaggacattgcattttcaggcttgtggtgataaataagatcgaccaatgcaagtgttcataatgactttccaattggccctgatgttctagcatgtgattacttcactcctggactgtgactttcagtgggagatggaagtttttcagagaactgaactgtggaaaaatgacctttccttaacttgaagctacttttaaaatttgagggtctggaccaaaagaagaggaatatcaggttgaagtcaagatgacagataaggtgagagtaatgactaactccaaagatggcttcactgaagaaaaggcattttaagattttttaaaaatcttgtcagaagatcccagaaaagttctaattttcattagcaattaataaagctatacatgcagaaatgaatacaacagaacactgctctttttgattttatttgtactttttggcctgggatatgggttttaaatggacattgtctgtaccagcttcattaaaataaacaatatttgtaaaaatcatactaatgcttattttattttaattgtatagaaagaaaaaaatgcctaaaataaggttttcttgcataaatactggaaattgcacatggtacaaattttttcttcattactgtacagtgatgatgttaatgactttgaagcactgaaagttactgaagtgccttctgaatcaaggatttaattaaggccacaatacctttttaatactcagtgttctgtttttttaaaaacttgatattcctgtatggtgcatatatgatacagttacctaatcatgttgaataaatgggcatgccaaaaatt

SEQ ID NO: 41 - DNA, 3419 bp, Homo sapiens
gcacgcacgcacgcacgccagcggccggcggggccgcaggctcgcgcccgggctcgccccgcgccgctccagaggctcgcgcactcagcaggttgggctgcggcggcggcggcagctgtggaagctcaggcgctgcgcgtgagaggtcccagatacgtctgcggttccggctccgccaccctcagcttctcttccccaggtctgggagccgagtgcggaaggagggaacggccctagctttgggaagccagaggacacccctggctcctgccgacaccgccctccttcccttcccagccgcgggcctcgctcggtgctaggctactctgccgggaggcggcggcggctgccagtctgtggagagtcctgctgccctccagccgggctcctccaccgggccttgcaggggccgagagagctcggtgcccgcccttccgctcgcctttttcgtcagctggctggagcagcatcggtccgggaggtctctaggctgaggcggcggccgctcctctagttccacaatgtccacgggcggagacttcgggaatccgctgaggaaattcaagctggtgttcctgggggagcaaagcgttggaaagacatctttgatcaccagattcatgtatgacagttttgacaacacctatcaggcaacaattggcattgactttttatcaaaaactatgtacttggaggatcgaacaatcaggcttcagctgtgggatactgcgggtcaggaacgtttccgtagcctcattcccagttacatccgtgattctgctgcagctgtagtagtttacgatatcacaaatgttaactcattccagcaaactacaaagtggattgatgatgtcagaacagaaagaggaagtgatgttatcatcatgctagtaggaaataaaacagatcttgctgacaagaggcaagtgtcaattgaggagggagagaggaaagccaaagagctgaatgttatgtttattgaaactagtgcaaaagctggatacaatgtaaagcagctctttcgacgtgtagcagcagctttgccgggaatggaaagcacacaggacagaagcagagaagatatgattgacataaaactggaaaagcctcaggagcaaccagtcagtgaaggaggctgttcctgctaatctcccatgtcatcttcaaccttcttcagaagctcactgctttggcccccttactctttcattgactgcagtgtgaatattggcttgaaccttttcccttcagtaataacgtattgcaattcatcattgctgcctgtctcgtggagatgatctattagcttcacaagcacaacaaaagtcagtgtcttcattatttatattttacaaaaagccaaaatatttcagcatattccagtgataactttaaaaattagatacattttcttaacatttttttcttttttaatgttatgataatgtacttcaaaatgatggaaatctcaacagtatgagtatggcttggttaacgagcggtatgttcacagcctactttatctctccttgcttttctcacctctcacttacccccattccctattaccctattcttacctagcctcccccgacttcctcaaaacaaacaagagatggcaaagcagcagttctaccaagcccattggaattatcctttaattttacagataccacttgctgtaggctacggaccaagatgtccaaaattattcttgagcactgatataaattacggtcttctttgaggtcaaaattcagccatcatggtaggcagtgcttgaatgagaaaaggctcctggtgcatcttcaaaatgagtcctaaagaacatactgagtacttagaagtagaagaacataagatgtatttctgactaaaacaaatggctctttcacatgtgctttattagactctgggagagaaaattaaccaagtgcttcagaacaggtttttagtatttaattcttcacggtaagaaaatgaagttctaatgaactgtttctcccaaggttttaaaattgtcaagagttattctgtttgtttaaaaaataagaaacctctttaagcaatagattttgcttgggttttcttttttaaaaacataatactgtgcaggcaaggcactgtaaaagttttaattccttccagaagaaccagtggaagaatttaaatttggcgctacgatcaaaactactgaattagtagaaataatgatgtctaaagcttaccaacaaaagaaccctcagcagaataacaaaaactttgctcaggacatttgaggtcaaattgaagacggaaaccggaaaccgttttcttgtaagcccctagaggcagatcaggtaaagcatacatagtagagggaaaggagagaatggaaataaaactcaatattatgcagatttatgccttattttttagcattttttaaggttgggtctttcaggctggttttggtttgtattagatctgtatagtttaattaactggtgatttagttttatatttaagctacaattaatcttttttctttggtgatatttatttctttgccttttttttttttaacaactttcaatcttcagatgtttcgttgaatctatttagagcttcaccatggcaatatgtatttcccttaaaacactgcaaacaaatatactaggagtgtgcccttttaatctttactagttattgtgagattgctgtgtaagctaataaacacatttgtaaatacattgtttgcaggacgaaaacttctgagttacagctcaggaaaagcctgctgaatttatgttgtaagcattacttaacacagtataaagatgaaaagacaacaaaaatatcttcatacttcctcatcccctcattggaacaaaaccttaaactgggagaaccttagtcccctctctttcctcttcctcctccacttcccacttattgtcaccttgtaatattcagagagcacttggattatggatctgaatagagaaatgcttacagataatcattagcccacataccagtaacttatacttaaagatgggatggagttgtaaagtgcttttataatacaatataattgttaaaggcaagggttgactctttgttttattttgacatggcatgtcctgaaataaatattgattcaatatggcagatgggtcatattctttatttggaagaagttgtgacttctgacatgggtgtgattgtcttcctacactgttgcatttgattctttttatgtatttttaagaaagtaaccagttatactgcttttaatattgattggtctttttatttggcttggagttcttcaaagcattgaagtgtgttcatagtccaggttttttttttaataaacacaattttgctgccaaaaatatataaataaaacacgaaagaaaacaaaaaaaaaaaaa

SEQ ID NO: 42 - DNA, 3527 bp, Homo sapiens
cttggctgatcgcgcctgcgcagtggggagcagctcgctcctgggctttgggctggctgcagtctgtctgagggcggccgaagtggctggctcatttaagatgaggcttctgctgcttctcctagtggcggcgtctgcgatggtccggagcgaggcctcggccaatctgggcggcgtgcccagcaagagattaaagatgcagtacgccacggggccgctgctcaagttccagatttgtgtttcctgaggttataggcgggtgtttgaggagtacatgcgggttattagccagcggtacccagacatccgcattgaaggagagaattacctccctcaaccaatatatagacacatagcatctttcctgtcagtcttcaaactagtattaataggcttaataattgttggcaaggatccttttgctttctttggcatgcaagctcctagcatctggcagtggggccaagaaaataaggtttatgcatgtatgatggttttcttcttgagcaacatgattgagaaccagtgtatgtcaacaggtgcatttgagataactttaaatgatgtacctgtgtggtctaagctggaatctggtcaccttccatccatgcaacaacttgttcaaattcttgacaatgaaatgaagctcaatgtgcatatggattcaatcccacaccatcgatcatagcaccacctatcagcactgaaaactcttttgcattaagggatcattgcaagagcagcgtgactgacattatgaaggcctgtactgaagacagcaagctgttagtacagaccagatgctttcttggcaggctcgttgtacctcttggaaaacctcaatgcaagatagtgtttcagtgctggcatattttggaattctgcacattcatggagtgcaataatactgtatagctttccccacctcccacaaaatcacccagttaatgtgtgtgtgtgttttttttttaaggtaaacattactacttgtaactttttttcttagtcatatttgaaaaagtagaaaattgagttacaatttgattttttttccaaagatgtctgttaaatctgttgtgcttttatatgaatatttgttttttatagtttaaaattgatcctttgggaatccagttgaagttcccaaatactttataagagtttatcagacatctctaatttggccatgtccagtttatacagtttacaaaatatagcagatgcaagattatgggggaaatcctatattcagagtactctataaatttttgtgtatgtgtgtatgtgcgtgtgattaccagagaactactaaaaaaaccaactgctttttaaatcctattgtgtagttaaagtgtcatgccttgaccaatctaatgaattgattaattaactgggcctttatacttaactaaataaaaaactaagcagatatgagttaaatttaaaagtttcaatttattgctcagtgtacctgttaacattatatttaacaattgcttaaatttttgtttttgatttatggataatttcttaagagtacacactttagatacacaaataatcgttcatttaccatctttaggatcattgaaactcatctcactaaagaaagttcacttgaacctctttatagcattgatactaggtgaacagaaattacctgactaataatttgtctaacatcatatatcagaattttattgtatatgatgaacaaaacttaaaattttttaaatttaatttttaaatactgtttcagagttctaaaaaggcagttttttaaaaaacttaagttgataaaaactgtaagaataatttagcagaaatagaaccagaatgtagaagagtagtcatgtaacagcagtaataacatacttcagcttccatataggaatagaagtggtagagccaaaagtgatttaggaaaagttataaggtacaggttgagtatcccttttccaaaaatgcttgggacaagaagtatttcagatttcataatttttttcaaagtttggaatatttgcattatacttaccagttgggcatcccaaatctgaaatctgaaatgttccatgagcatttcctttgagtgtcatgttggcactcaaaaaggttcaacattgagtccacttaacacttaggtgttagaagacctaactttctgtaacaattaaccttatactttgtttgtcatcgaatatttgttgaatgcatgtcaggtaatggtcttgattgtgatagcttcaaggtggaacatactgtaatctccagatgctaggaagttagtctaataattcactgcagaaaattgattaagtggctgtccttttaattaagagtgtggagtcataaacttaagttcttcatatagtgacaagagtccttagagattgttattcaagttccttagaaattgttatttaggtataatatcatcttgtctttgactagagcttgaaaccttgttatctgattgtgtaccactccaaattccctgccttctgcaagttgaatgtcttgctgaatgtgtctaggggttcatcttcagtaatcgacattccactagtgccatagttaacttcatgacatgtagacattcaaaacttgagccttggatgttcctgtggacctgacagttaaaaatataaagaacctaggattcaattccaactttctctgtttgccttgggttgaataacttatcttttggagaatagctttaagtggcttagacactgataaaattcagctgtgttgttgacgctcatctcttttgtcttacgcttagccatatttaaatcttgaatttaatagagtctagtgaaaaaaatgagtgggaagaatgaatataaaagtaataatataaggaaaaagggaaagtaaactatttagaatgtagttttgttatattcccagcatttcaatatttattagttacttgtaaattactgtggctgtgtagtttataaatgtctgtgcactatattaattagaagaccatagaacatgccagcaggttggctaatgctatgggggtttttaccacagttgccattgtggaagaaattatttggtacattaataaaaaaagttggtaaaacatggttttatacctcagtgtataagatgtgcaagacaaatatgcttatttccttttctagaatataagtgatattatttgcttatgacactaacactattaatgacaggagtcaatcagcctttacagctatcaaaatataatgagatcccaatgatgattcttttttactttgaatgttaattagtttgggactttgattggctggcaaacattttatcattgtcagaatttaatttagatttcaaaaatagcttacaggattttaaacatggtgtggtattctaaagccttttttttaaaaaaagagatctttttgagagaaacaaatgaggattgtaaagtttggggacttacctctgtagcattgtgaaaataaactttgattaagctgatttgaaaggaaaaaaaaaaaaaaaaaaaaaaa